(19) United States
(12) Patent Application Publication
(10) Pub. No.: US 2024/0189240 A1
(43) Pub. Date: Jun. 13, 2024

(54) PHARMACEUTICAL COMPOSITIONS COMPRISING ZOLMITRIPTAN

(71) Applicant: MapLight Therapeutics, Inc., San Francisco, CA (US)

(72) Inventors: Michael WOOD, Gloucester, MA (US); James LILLIE, Wellesley, MA (US)

(21) Appl. No.: 18/552,420

(22) PCT Filed: Mar. 24, 2022
(86) PCT No.: PCT/US2022/021738

§ 371 Date: Sep. 25, 2023

Related U.S. Application Data
(60) Provisional application No. 63/165,938, filed on Mar. 25, 2021.
(60) Provisional application No. 63/306,377, filed on Feb. 3, 2022.

(51) Int. Cl. A61K 9/20; A61K 31/422; A61P 25/00

(52) U.S. Cl. CPC A61K 9/2086; A61K 31/422; A61P 25/00

(57) ABSTRACT

Provided herein are oral compositions comprising zolmitriptan, and methods of use thereof e.g., for the treatment of the symptoms of autism spectrum disorder, and the treatment of aggression in patients with dementia.

### CROSS-REFERENCE TO RELATED APPLICATIONS

This application claims priority to U.S. Provisional Application No. 63/306,377, filed on Feb. 3, 2022 and U.S. Provisional Application No. 63/165,938 filed on Mar. 25, 2021, and the disclosures of which are hereby incorporated by reference in their entireties for all purposes.

### FIELD OF THE DISCLOSURE

The present disclosure relates to pharmaceutical compositions of zolmitriptan and their use.

### BACKGROUND OF THE DISCLOSURE

Zolmitriptan is a triptan whose molecular target is the serotonin (5-HT) 1B receptors expressed in intracranial arteries and 5-HT 1D receptors located on peripheral trigeminal sensory nerve terminals in the meninges and central terminals in brainstem sensory nuclei. The chemical name of zolmitriptan is (4S)-4-[[3-[2-(dimethylamino)ethyl]-1H-indol-5-yl]methyl]-1,3-oxazolidin-2-one and its structural formula is shown below.

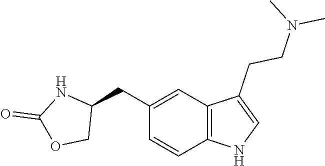 [SMILES: CN(C)CCc1c[nH]c2ccc(C[C@H]3COC(=O)N3)cc12]

Zolmitriptan is marketed as ZOMIG® in the United States and other markets for the treatment of headaches, such as migraines.

Zolmitriptan can be used to treat the symptoms associated with autism spectrum disorder (ASD). ASD patients commonly exhibit sensory processing abnormalities and may have aversions to the color, taste, smell, and/or texture of foods or medicines. Thus, maintaining medication adherence in ASD patients can be challenging.

Thus, there is a need for pharmaceutical compositions of zolmitriptan that provide therapeutic effectiveness when administered once-a-day, twice-a-day, or three-times-a-day.

### SUMMARY OF THE DISCLOSURE

In one aspect, the present disclosure provides pharmaceutical compositions comprising zolmitriptan or a pharmaceutically acceptable salt thereof. In some embodiments, the disclosure provides pharmaceutical compositions that provide therapeutically effective zolmitriptan blood plasma levels when administered on a once a day or twice a day basis.

In some embodiments, the compositions comprise zolmitriptan in an amount of from about 7.5 mg to about 150 mg. In some embodiments, the compositions comprise zolmitriptan in an amount of from about 60 mg to about 120 mg. In some embodiments, the pharmaceutical compositions comprise about 7.5 mg, about 10 mg, about 15 mg, about 20 mg, about 25 mg, about 50 mg, about 75 mg, about 100 mg, about 125 mg, or about 150 mg of zolmitriptan.

In some embodiments, the present disclosure provides a composition for treating the symptoms of autism spectrum disorder, wherein the composition comprises from about 7.5 mg to about 150 mg of zolmitriptan, or a pharmaceutical acceptable salt thereof, wherein the administration of the composition provides a therapeutically effective plasma concentration of zolmitriptan for a period of at least about 12 h.

In some embodiments, the present disclosure provides compositions for treating the symptoms of autism spectrum disorder, wherein the composition comprises zolmitriptan, or a pharmaceutically acceptable salt thereof, wherein the oral administration of the composition provides an AUC_{0-24h,ss} of about 10 ng·h/mL to about 1150 ng·h/mL over a period of about 12-24 h to treat the symptoms of autism spectrum disorder.

In some embodiments, the present disclosure provides compositions for treating the symptoms of autism spectrum disorder, wherein the composition comprises zolmitriptan or a pharmaceutically acceptable salt thereof, wherein the injection of the composition provides an AUC_{0-24h,ss} of about 10 ng·h/mL to about 1150 ng·h/mL over a period of about 1 day to about 1 month to treat the symptoms of autism spectrum disorder.

In some embodiments, the present disclosure provides methods of treating the symptoms of autism spectrum disorder, comprising administering a pharmaceutical composition to a patient in need thereof, wherein the pharmaceutical composition comprises about 2 mg to about 150 mg of zolmitriptan, or a pharmaceutical acceptable salt thereof, wherein the administration provides a therapeutically effective plasma concentration for period of at least about 12 h.

In some embodiments, the present disclosure provides a composition for treating aggression in patients with dementia (e.g., Alzheimer's patients), wherein the composition comprises from about 7.5 mg to about 150 mg of zolmitriptan, or a pharmaceutical acceptable salt thereof, wherein the administration of the composition provides a therapeutically effective plasma concentration of zolmitriptan for a period of at least about 12 h.

In some embodiments, the present disclosure provides a composition for treating aggression in patients with dementia (e.g., Alzheimer's patients), wherein the composition comprises zolmitriptan, or a pharmaceutically acceptable salt thereof, wherein the oral administration of the composition provides an AUC_{0-24h,ss} of about 10 ng·h/mL to about 1150 ng·h/mL over a period of about 12-24 h to treat the symptoms of autism spectrum disorder.

In some embodiments, the present disclosure provides a composition for treating aggression in patients with dementia (e.g., Alzheimer's patients), wherein the composition comprises zolmitriptan or a pharmaceutically acceptable salt thereof, wherein the injection of the composition provides an AUC_{0-24h,ss} of about 10 ng·h/mL to about 1150 ng·h/mL over a period of about 1 day to about 1 month to treat the symptoms of autism spectrum disorder.

In some embodiments, the present disclosure provides methods of treating aggression in patients with dementia (e.g., Alzheimer's patients), comprising administering a pharmaceutical composition to a patient in need thereof, wherein the pharmaceutical composition comprises about 2 mg to about 150 mg of zolmitriptan, or a pharmaceutical acceptable salt thereof, wherein the administration provides a therapeutically effective plasma concentration for period of at least about 12 h.

### BRIEF DESCRIPTION OF THE DRAWINGS

FIG. 1 shows the attack time (secs) of adult male CD-1 mice from a cross-over Resident-Intruder (RI) study in which resident CD-1 mice were treated with vehicle control, 10 mg/kg zolmitriptan or 0.03 mg/kg risperidone. Attack time was measured over a 5 min period as a cross over design. Data are expressed as mean f standard error of means.

FIG. 2 shows the sociability index in a valproic acid (VPA)-induced c57/Bl6 mouse model of Autism Spectrum Disorder in mice treated with vehicle control or 10 mg/kg zolmitriptan.

FIG. 3 shows the average CSF levels of zolmitriptan and its metabolite N-desmethyl zolmitriptan (NDMZ) following oral administration of 5 mg, 10 mg, 20 mg, and 30 mg doses to human subjects.

FIG. 4 shows zolmitriptan dissolution profiles for extended release tablets (15 mg ER zolmitriptan) and immediate release/extended release 25 mg (10 mg IR zolmitriptan/15 mg ER zolmitriptan) bilayer tablets.

FIG. 5 shows zolmitriptan dissolution profiles for 12 mg (3 mg IR/9 mg ER) and 24 mg (6 mg IR/18 mg ER) zolmitriptan immediate release/extended release oral bilayer tablets (n=6)

FIG. 6 shows Study Design Schematic for the Study described in Example 10.

### DEFINITIONS

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound, or a salt, solvate or ester thereof, that, when administered to a patient, is capable of performing the intended result. For example, in some embodiments, an effective amount of zolmitriptan is that amount that is required to reduce at least one symptom of ASD in a patient. The actual amount that comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid.

The term “pharmaceutically acceptable salts” includes those obtained by reacting the active compound functioning as a base, with an inorganic or organic acid to form a salt, for example, salts of 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid, (−L) malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, propionic acid, pyroglutamic acid (−L), salicylic acid, sebacic acid, stearic acid, succinic acid, sulfuric acid, tartaric acid (+L), thiocyanic acid, toluenesulfonic acid (p), and undecylenic acid. Those skilled in the art will further recognize that acid addition salts may be prepared by reaction of the compounds with the appropriate inorganic or organic acid via any of a number of known methods.

The term “treating” as used herein with regard to a patient, refers to improving at least one symptom of the patient's disorder. Treating can be, improving, or at least partially ameliorating a disorder.

The term “therapeutic effect” as used herein refers to a desired or beneficial effect provided by the method and/or the composition. For example, in some embodiments, the method for treating autism spectrum disorder provides a therapeutic effect when the method improves at least one symptom of ASD, e.g., an improvement in irritability associated with ASD or increased sociability, in a patient. In some embodiments, the method for treating aggression in a dementia patient (e.g., an Alzheimer's patient) provides a therapeutic effect when the method improves at least one symptom of the patient's aggression in a patient.

### DETAILED DESCRIPTION OF THE DISCLOSURE

The present disclosure provides pharmaceutical compositions comprising zolmitriptan or a pharmaceutically acceptable salt thereof. In some embodiments, the pharmaceutical compositions provide therapeutically effective blood plasma levels when administered on a once-a-day, twice-a-day, or three-times-a-day basis. In some embodiments, the pharmaceutical compositions achieve similar therapeutic efficacy to the reference listed product (ZOMIG®), but have an improved safety profile.

### Zolmitriptan

Zolmitriptan as employed in the present methods can form a part of a pharmaceutical composition by combining zolmitriptan, or a pharmaceutically acceptable salt thereof, with a pharmaceutically acceptable carrier. Additionally, the compositions can include an additive selected from the group consisting of adjuvants, excipients, diluents, release-modifying agents and stabilizers.

Zolmitriptan is a type of triptan. Triptans belong to the serotonin receptor subtype-selective drug class. Triptans have selective activity on the 5-HT_{1B}, 5-HT_{1D}, and 5-HT_{1F} receptors, which belong to the serotonin (5-HT) system. Triptans exhibit vasoconstrictive properties, which are mediated by an action on 5-HT_{1B} in arterial smooth muscle. Vasoconstriction by triptans leads to a dose-dependent increase in blood flow velocity in middle cerebral vessels. It is thought that triptans inhibit the abnormal activation of peripheral nociceptors. Triptans are presumed to reduce plasma protein extravasation (PPE) by inhibiting the activation of nociceptors and preventing the peripheral release of vasoactive peptides, including substance P and calcitonin gene related peptide (CGRP). Triptan binding sites also exist within the central nervous system. Thus, it is possible that triptans exhibit an effect on the central nervous system. At the time of this disclosure, triptans are FDA approved for the treatment of migraine headaches.

The serotonin system has been implicated in the pathophysiology of autism since an abnormal blood 5-HT level was discovered as the first biomarker of the disorder more than 50 years ago (Schain et al. J Pediatr. 1961 March; 58:315-20). A major source of 5-HT in the brain originates from the midbrain dorsal raphe nucleus (DRN) that projects broadly across the cortex, amygdala, hypothalamus, and to other subcortical structures associated with emotional processing and social behavior. Dense serotonergic projections from the DRN innervate the nucleus accumbens (NAc), a well-conserved basal forebrain structure that acts as the integrator of motivational signals preceding the selection of behavioral actions (Kravitz et al. Physiology (Bethesda). 2012 June; 27(3):167-77.). In the context of social interaction, NAc reward processing is thought to govern the choice of social approach vs. social avoidance behaviors (Pfaff. Trends Neurosci. 2019 July; 42(7):448-457). Evidence from preclinical studies supports a critical role for 5-HT signaling in social behaviors and social reward (Kane et al. PLoS One. 2012; 7(11):e48975.; Challis et al. J Neurosci. 2013 Aug. 28; 33(35):13978-88, 13988a.; Li et al. Nat Commun. 2016 Jan. 28; 7:10503.). Furthermore, aberrant processing of rewards in the NAc has been observed in fMRI imaging studies of children with ASD (Clements et al. JAMA Psychiatry. 2018 Aug. 1; 75(8):797-808. doi: 10.1001/jamapsychiatry.2018.1100.).

Within the human brain, 5-HT1B is highly expressed in the NAc (Garcia-Alloza et al. Neuropsychologia. 2005; 43(3):442-9.; Varnas et al. Hum Brain Mapp. 2004 July; 22(3):246-60.; Varnas et al. Synapse. 56: 21-8.). It is an inhibitory G protein coupled receptor localized in axon terminals that functions to suppress neurotransmitter release (Sari. Neurosci Biobehav Rev. 2004 October; 28(6):565-82.). As an autoreceptor, 5-HT1B inhibits the release of 5-HT, but it also plays an important role as a heteroreceptor to inhibit the release of other neurotransmitters including glutamate, GABA, dopamine and acetylcholine. (Boscher et al. (1994) Neuroscience 58:167-182.). According to the present disclosure, the 5-HT1B agonism of the triptans described herein modulates the behavior symptoms of ASD.

The synthesis of zolmitriptan is described in U.S. Pat. No. 9,006,453, which is hereby incorporated by reference in its entirety for all purposes. The synthesis of optically pure zolmitriptan is described in International Publication No. 2005/105792, which is hereby incorporated by reference in its entirety for all purposes.

In some embodiments, the zolmitriptan used in the compositions and methods of the present disclosure is a pharmaceutically acceptable salt of zolmitriptan. In some embodiments, the pharmaceutically acceptable salt of zolmitriptan used in the formulations and methods of the present disclosure is selected from the group consisting of oxalate, camphorsulfonate, sulfonate, hydrobromide, hydrochloride, hydrogensulfate, mesylate, succinate and tartrate.

### Pharmaceutical Compositions

In one aspect, the present disclosure provides pharmaceutical compositions comprising zolmitriptan.

In some embodiments, the present disclosure provides pharmaceutical compositions that provide therapeutic effects for the treatment of one or more of the symptoms associated with ASD when administered on a once-daily, twice-daily or three-times daily basis.

In some embodiments, the present disclosure provides pharmaceutical compositions that provide therapeutic effects for the treatment of aggression in patients with dementia (e.g., Alzheimer's patients) when administered on a once-daily, twice-daily or three-times daily basis.

In some embodiments, the pharmaceutical compositions provided herein are administered to the subject in a fasted state. In some embodiments, the pharmaceutical compositions are administered to the subject in a fed state.

In some embodiments, the pharmaceutical compositions described herein comprise zolmitriptan in an amount of from about 1 mg to about 200 mg, e.g., about 1 mg, about 3 mg, about 5 mg, about 7.5 mg, about 10 mg, about 12 mg about 20 mg, about 24 mg, about 30 mg, about 36 mg, about 40 mg, about 48 mg, about 50 mg, about 60 mg, about 70 mg, about 72 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg, including all values and subranges therebetween. In some embodiments, the pharmaceutical compositions described herein comprise zolmitriptan in an amount of from about 50 mg to about 200 mg. In some embodiments, the pharmaceutical compositions described herein comprise about 50 mg, about 75 mg, about 100 mg, about 125 mg, about 150 mg, about 175 mg or about 200 mg of zolmitriptan. In some embodiments, the pharmaceutical compositions described herein comprise zolmitriptan or a pharmaceutically acceptable salt thereof in an amount from about 7.5 mg to about 50 mg. In some embodiments, the pharmaceutical compositions described herein comprise zolmitriptan or a pharmaceutically acceptable salt thereof in an amount from about 10 mg to about 30 mg. In some embodiments, the pharmaceutical compositions described herein comprise about 12 mg, about 24 mg, about 36 mg, or about 48 mg of zolmitriptan or a pharmaceutically acceptable salt thereof. In embodiments, the pharmaceutical compositions described herein comprise about 12 mg, about 24 mg, about 36 mg, about 48, or about 72 mg of zolmitriptan or a pharmaceutically acceptable salt thereof.

In some embodiments, the pharmaceutical compositions release zolmitriptan in two or more pulses, e.g., two, three, four, five, six, seven, eight, nine, and ten or more pulses. The timing of the pulses can be modified to release the drug in the desired area of the gastrointestinal tract. For example, in some embodiments, the pharmaceutical compositions described herein are formulated to release one or more pulses in the stomach, and one or more pulses in the intestines.

In some embodiments, the pharmaceutical compositions described herein comprise (i) an immediate release component and (ii) a delayed release component, each of which comprise a portion of the total amount of zolmitriptan in the pharmaceutical composition.

In some embodiments, the pharmaceutical compositions described herein comprise (i) an immediate release component and (ii) an extended release component, each of which comprise a portion of the total amount of zolmitriptan in the pharmaceutical composition.

In some embodiments, the ratio of zolmitriptan in the immediate release component compared to the delayed or extended release component is in the range of from about 1:99 to about 99:1, e.g., about 1:99, about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, and about 99:1, including all values and subranges therebetween. In some embodiments, the ratio of zolmitriptan in the immediate release component compared to the delayed or extended release component is about 25:75. In some embodiments, the immediate release component comprises up to about 75% of the total amount of zolmitriptan in the pharmaceutical composition (e.g., about 10%, about 15%, about 20%, about 25%, about 30%, about 35%, about 40%, about 45%, about 50%, about 55%, about 60%, about 65%, about 70%, and about 74% including all values and subranges therebetween), and the delayed or extended release component comprises about 25% or more of the total amount of zolmitriptan in the pharmaceutical composition (e.g., about 25%, about 20%, about 35%, about 40%, about 45%, about 50%, about 55%, about 60%, about 65%, about 70%, about 75%, about 80%, about 85%, about 90%, about 95%, and about 99%, including all values and subranges therebetween). In some embodiments, the immediate release component comprises about 20%-40% by weight of the total zolmitriptan in the composition, including about 20%, about 25%, about 30%, about 35%, to about 40%, including all values and subranges therebetween by weight of the total zolmitriptan in the composition. In some embodiments, the extended release component contains about 60%-80% by weight of the total zolmitriptan in the composition, including about 60%, about 65%, about 70%, about 75%, to about 80% including all values and subranges therebetween by weight of the total zolmitriptan in the composition. In some embodiments, the immediate release component contains about 25% of the total zolmitriptan in the composition and the extended release component contains about 75% of the total zolmitriptan in the composition.

In some embodiments, the immediate release component comprises about 1 mg to about 20 mg of zolmitriptan or a pharmaceutically acceptable salt thereof, including about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, to about 20 mg, including all values and subranges therebetween. In some embodiments, the immediate release component comprises about 1 mg to about 10 mg, or about 3 mg to about 10 mg of zolmitriptan or a pharmaceutically acceptable salt thereof. In some embodiments, the immediate release component comprises about 3 mg of zolmitriptan or a pharmaceutically acceptable salt thereof. In some embodiments, the immediate release component comprises about 6 mg of zolmitriptan or a pharmaceutically acceptable salt thereof.

In some embodiments, the extended release component comprises about 1 mg to about 50 mg of zolmitriptan or a pharmaceutically acceptable salt thereof, including about 1 mg, about 2 mg, about 3 mg, about 4 mg, 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, and about 50 mg, including all values and subranges therebetween. In some embodiments, the extended release component comprises about 5 mg to about 25 mg of zolmitriptan or a pharmaceutically acceptable salt thereof. In some embodiments, the extended release component comprises about 9 mg of zolmitriptan or a pharmaceutically acceptable salt thereof. In some embodiments, the extended release component comprises about 18 mg of zolmitriptan or a pharmaceutically acceptable salt thereof.

In some embodiments, the immediate release component comprises about 0.01-10% w/w of zolmitriptan or a pharmaceutically acceptable salt thereof, based on the total weight of the composition (e.g., bilayer tablet), including from about 0.01% w/w, about 0.02% w/w, about 0.03% w/w, about 0.04% w/w, about 0.05% w/w, about 0.06% w/w, about 0.07% w/w, about 0.08% w/w, about 0.09% w/w, 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 1.5%, about 2% w/w, about 2.5% w/w, about 3% w/w, about 3.5% w/w, about 4% w/w, about 4.5% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w and about 10% w/w of zolmitriptan or a pharmaceutically acceptable salt thereof, based on the total weight of the composition including all values and subranges therebetween. In some embodiments, the immediate release component comprises about 0.01-5% w/w, or about 0.1-2.5% w/w of zolmitriptan or a pharmaceutically acceptable salt thereof, based on the total weight of the composition. In some embodiments, the extended release component comprises about 0.05-20% w/w of zolmitriptan or a pharmaceutically acceptable salt thereof, based on the total weight of the composition (e.g., bilayer tablet), including from about 0.05% w/w, about 0.06% w/w, about 0.07% w/w, about 0.08% w/w, about 0.09% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 1.5%, about 2% w/w, about 2.5% w/w, about 3% w/w, about 3.5% w/w, about 4% w/w, about 4.5% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w, about 11% w/w, about 12% w/w, about 13% w/w, about 14% w/w, about 15% w/w, about 16% w/w, about 17% w/w, about 18% w/w, about 19% w/w, and about 20% w/w including all values and subranges therebetween of the total weight of the composition. In some embodiments, the extended release component comprises about 1-10% w/w, or 0.05-5% w/w, or 0.01-2.5% w/w of zolmitriptan or a pharmaceutically acceptable salt thereof, based on the total weight of the composition.

Non-limiting examples of suitable immediate release components include one or more immediate release components incorporated as a portion or portions of a dosage form that can be in the form of tablets, liquid compositions (e.g., suspension and emulsions), capsules, elixirs, syrups, drug containing beads and particles, and the like. In some embodiments, the immediate release component can be disposed over the top of the delayed or extended release component, or the immediate release component can be distinct from the delayed or extended release component. For example, an immediate release component can be in the form of a coating that can be disposed over an osmotic delivery system or a capsule, or immediate release beads can be combined with delayed or extended release beads in a capsule or tablet. In other embodiments, the immediate release component can be provided as a layer of a bi- or multi-layer tablet. In some embodiments the immediate release component is provided as a layer of a bilayer tablet. The delayed release component can be any formulation that substantially prevents release of a portion (e.g., about 25-99%) of the total amount of the zolmitriptan in the pharmaceutical composition for at least 30 minutes. Non-limiting examples of suitable delayed release components include one or more delayed release components incorporated as a portion or portions of a drug-containing tablet, particle, bead, and the like, coated with a delayed release polymer, a delayed release matrix comprising zolmitriptan, and an osmotic pump comprising zolmitriptan.

In some embodiments, the composition comprises one or more immediate release components in combination with one or more extended release components, where the extended release components release the zolmitriptan over an extended period of time.

In some embodiments, the composition is a multiparticulate formulation. In some embodiments, the composition is a gastrorentive tablet.

In some embodiments, the extended release component is a gastrorentive layer. In some embodiments, the composition is gastroretained for at least about 1 h, at least about 2 h, at least about 3 h, at least about 4 h, at least about 5 h, at least about 6 h, at least about 7 h, at least about 8 h, at least about 9 h, at least about 10 h, at least about 11 h, or at least about 12 h following oral administration. In some embodiments, the composition is gastroretained for at least about 2 h following oral administration. In some embodiments, the composition is gastroretained for at least about 8 h following oral administration.

The present disclosure provides enumerated embodiments disclosing compositions and methods for treating the symptoms of autism spectrum disorder. The compositions and methods described herein are also suitable for treating other conditions characterized by aggression (e.g., aggression in dementia patients such as Alzheimer's patients). Thus, the enumerated embodiments described herein for the compositions and treatment of symptoms of autism spectrum disorder equally apply for the treatment of conditions characterized by aggression (e.g., aggression in dementia patients such as Alzheimer's patients).

In some embodiments, provided herein is an oral composition for treating the symptoms of autism spectrum disorder, wherein the composition comprises from about 7.5 mg to about 90 mg of zolmitriptan, or a pharmaceutical acceptable salt thereof, and the oral administration of the composition provides a therapeutically effective plasma concentration of zolmitriptan for a period of at least 8 hours.

In some embodiments, provided herein is an oral composition for treating aggression in patients with dementia, wherein the composition comprises from about 7.5 mg to about 90 mg of zolmitriptan, or a pharmaceutical acceptable salt thereof, and the oral administration of the composition provides a therapeutically effective plasma concentration of zolmitriptan for a period of at least 8 hours.

In some embodiments, provided herein is an oral composition for treating aggression in Alzheimer's patients, wherein the composition comprises from about 7.5 mg to about 90 mg of zolmitriptan, or a pharmaceutical acceptable salt thereof, and the oral administration of the composition provides a therapeutically effective plasma concentration of zolmitriptan for a period of at least 8 hours.

In some embodiments, the pharmaceutical compositions of the present disclosure when dissolution tested using United States Pharmacopoeia Apparatus II (paddles @ 50 rpm) in 900 mL of 0.1N HCl (or a suitable medium) at about 37° C., exhibits a zolmitriptan release profile substantially corresponding to the following pattern:
from about 20%-40% of the total zolmitriptan is released after about 15 minutes;
after about 4 h about 40%-75% of the total zolmitriptan is released; and
after about 8 h about 75%-90% of the total zolmitriptan is released.

In some embodiments, the dissolution is tested under simulated fasted conditions. In some embodiments, the dissolution is tested under simulated fed conditions.

In some embodiments, when dissolution of the pharmaceutical compositions provided herein is tested using United States Pharmacopoeia Apparatus II (paddles @ 50 rpm) in 900 mL of 0.1N HCl (or a suitable medium) at about 37° C., about 5%-40% of the total zolmitriptan is released after about 15 minutes, including about 5%, about 10%, about 15%, about 20%, about 25%, about 30%, about 35%, and about 40% of the total zolmitriptan is released after about 15 minutes, including all values and subranges therebetween.

In some embodiments, when dissolution of the pharmaceutical compositions provided herein is tested using United States Pharmacopoeia Apparatus II (paddles @ 50 rpm) in 900 mL of 0.1N HCl (or a suitable medium) at about 37° C., after about 4 h about 40%-75% of the total zolmitriptan is released, including about 40%, about 45%, about 50%, about 55%, about 60%, about 65%, about 70%, and about 75% of the total zolmitriptan is released after about 4 h, including all values and subranges therebetween.

In some embodiments, when dissolution of the pharmaceutical compositions provided herein is tested using United States Pharmacopoeia Apparatus II (paddles @ 50 rpm) in 900 mL of 0.1N HCl (or a suitable medium) at about 37° C., after about 8 h about 75%-90% of the total zolmitriptan is released, including about 75%, about 80%, about 85%, and about 90%, including all values and subranges therebetween. In some embodiments, about 85% of the total zolmitriptan is released within 9-10 hours. In some embodiments, the dissolution is tested under simulated fed conditions.

In embodiments, the pharmaceutical composition thereby provides a therapeutically effective plasma concentration over an extended period of time, typically over a period of about 6-24 hours (e.g., about 8 hours, or about 12 hours, or about 16 hours) to treat the symptoms of autism spectrum disorder in humans.

In embodiments, the pharmaceutical composition thereby provides a therapeutically effective plasma concentration over an extended period of time, typically over a period of at least about 6-24 hours (e.g., at least about 8 hours, at least about 12 hours, or at least about 16 hours) to treat the symptoms of autism spectrum disorder in humans.

In embodiments, the pharmaceutical composition thereby provides a therapeutically effective plasma concentration over an extended period of time, typically over a period of about 6-24 hours (e.g., about 8 hours, or about 12 hours, or about 16 hours) to treat aggression in patients with dementia (e.g., Alzheimer's patients).

In embodiments, the pharmaceutical composition thereby provides a therapeutically effective plasma concentration over an extended period of time, typically over a period of at least about 6-24 hours (e.g., at least about 8 hours, at least about 12 hours, or at least about 16 hours) to treat aggression in patients with dementia (e.g., Alzheimer's patients).

In some embodiments, the pharmaceutical compositions of the present disclosure, including the one or more immediate release component(s) and/or the delayed or extended release component, comprise at least one pharmaceutically acceptable carrier, diluent, and/or excipient. Pharmaceutically acceptable carriers, diluents or excipients include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier.

In some embodiments, the extended release component is a gastroretentive layer and the gastroretentive layer comprises zolmitriptan or a pharmaceutically acceptable salt thereof, a drug release modifier, and optionally one or more excipients described herein, such a lubricant, and a binder and/or a diluent, as well as other excipients. In some embodiments, the gastro-retentive layer comprises one or more drug release modifiers include any swellable, erodible hydrophilic polymers disclosed herein, such as water-swellable polymers (e.g., polyethylene oxide). In some embodiments, the gastro-retentive layer comprises a drug release modifier (e.g., a water-swellable polymer) in about 15% to about 75% w/w of the total composition (e.g., bilayer tablet) including about 15% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, and about 75% w/w of the total composition, including all values and subranges therebetween. In some embodiments, the gastro-retentive layer comprises a drug release modifier (e.g., a water-swellable polymer, such as polyethylene oxide) about 35-55% w/w of the total composition (e.g., bilayer tablet). Suitable lubricants for the gastro-retentive layer include any lubricants listed herein, such as stearic acid, and stearic acid salts, for example magnesium stearate. In some embodiments, the gastro-retentive layer comprises from 0.05 to 5% w/w lubricant, based on the total weight of the composition (e.g., bilayer tablet) including e.g., about 0.05% w/w, about 0.06% w/w, about 0.07% w/w, about 0.08% w/w, about 0.09% w/w, about 0.1% w/w, about 0.2% w/w, about 0.3% w/w, about 0.4% w/w. about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, and about 5% w/w including all values and subranges therebetween. Suitable binders for gastro-retentive layer include any binder listed herein, including microcrystalline cellulose, such as Avicel™ PH101, or Ceolus™ KG802. The amount of binder in the gastroretentive layer may range from about 10 to about 50% w/w, based on the total weight of the composition (e.g., bilayer tablet) including e.g., about 10% w/w, about 15% w/w, about 20% w/w, about 25% w/w, about 30% w/w, about 35% w/w, about 40% w/w, and about 45% w/w, including all values and subranges therebetween.

In some embodiments, suitable pharmaceutically acceptable carriers include, but are not limited to, inert solid fillers or diluents and sterile aqueous or organic solutions. Pharmaceutically acceptable carriers are well known to those skilled in the art and include, but are not limited to, aqueous and non-aqueous solutions. Pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate. Aqueous carriers suitable for use in the present application include, but are not limited to, water, ethanol, alcoholic/aqueous solutions, glycerol, emulsions or suspensions, including saline and buffered media.

Liquid carriers suitable for use in the present application include, but are not limited to, water (partially containing additives, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil).

Liquid carriers suitable for use in the present application can be used in preparing solutions, suspensions, emulsions, syrups, elixirs and pressurized compounds. In some embodiments, the active ingredient is dissolved or suspended in a pharmaceutically acceptable liquid carrier such as water, an organic solvent, a mixture of both or pharmaceutically acceptable oils or fats. In some embodiments, the liquid carrier contains other suitable pharmaceutical additives such as solubilizers, emulsifiers, buffers, preservatives, sweeteners, flavoring agents, suspending agents, thickening agents, colors, viscosity regulators, stabilizers or osmo-regulators.

Solid carriers suitable for use in the present application include, but are not limited to, inactive substances such as lactose, starch, glucose, methylcellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid that is in admixture with the finely divided active compound. In tablets, the active compound is mixed with a carrier having the necessary compression properties in suitable proportions and compacted in the shape and size desired. The powders and tablets may contain up to 99% of the active compound. Suitable solid carriers include, for example, calcium phosphate, magnesium stearate, talc, sugars, lactose, dextrin, starch, gelatin, cellulose, polyvinylpyrrolidone, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide delayed or controlled release of the active ingredient therein using, for example, hydroxypropyl methylcellulose in varying proportions to provide the desired release profile. Tablets may optionally be provided with an enteric coating, to provide release in parts of the gut other than the stomach.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations described herein. Diluents increase the bulk of a solid pharmaceutical composition and/or combination, and may make a pharmaceutical dosage form containing the composition and/or combination easier for the patient and care giver to handle. In various embodiments, diluents for solid compositions include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc, and/or mixtures of any of the foregoing. Specific examples of: microcrystalline cellulose include those sold under the Trademark Avicel (FMC Corp., Philadelphia, Pa.), for example, Avicel™ pH101, Avicel™ pH102 and Avicel™ pH112; lactose includes lactose monohydrate, lactose anhydrous and Pharmatose DCL21; dibasic calcium phosphate includes Emcompress.

Lubricants are used to facilitate tablet manufacture, promoting powder flow and preventing particle capping (i.e., particle breakage) when pressure is relieved. Useful lubricants are magnesium stearate, calcium stearate, stearic acid, glyceryl behenate, talc, colloidal silicon dioxide such as Aerosil™ 200, mineral oil (in PEG), hydrogenated vegetable oil (e.g., comprised of hydrogenated and refined triglycerides of stearic and palmitic acids), combinations thereof.

Binders are used to impart cohesive qualities to a tablet, and thus ensure that the tablet or tablet layer remains intact after compression. Suitable binder materials include, but are not limited to, starch (including corn starch and pregelatinized starch), gelatin, sugars (including sucrose, glucose, dextrose and lactose), polyethylene glycol, polyvinyl alcohol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum, and combinations thereof. Examples of polyvinylpyrrolidone include povidone, copovidone and crospovidone.

Fillers include, for example, materials such as silicon dioxide, titanium dioxide, alumina, talc, kaolin, powdered cellulose, microcrystalline cellulose, urea, sodium chloride, as well as saccharides, or combinations thereof. Any suitable saccharide may be used in the composition of the present disclosure. As used herein, the “saccharides” used in the invention include sugar alcohols, monosaccharides, disaccharides, and oligosaccharides. Exemplary sugar alcohols include, but are not limited to, xylitol, mannitol, sorbitol, erythritol, lactitol, pentitol, and hexitol. Exemplary monosaccharides include, but are not limited to, glucose, fructose, aldose and ketose. Exemplary disaccharides include, but are not limited to, sucrose, isomalt, lactose, trehalose, and maltose. Exemplary oligosaccharides include, but are not limited to, fructo-oligosaccharides, inulin, galacto-oligosaccharides, and mannan-oligosaccharides. In some embodiments, the saccharide is sorbitol, mannitol, or xylitol. In some embodiments, the saccharide is sorbitol. In some embodiments, the saccharide is sucrose.

Disintegrants are used to facilitate disintegration of the tablet, thereby increasing the erosion rate relative to the dissolution rate, and are generally starches, clays, celluloses, algins, gums, or crosslinked polymers (e.g., crosslinked polyvinyl pyrrolidone). Other non-limiting examples of suitable disintegrants include, for example, lightly crosslinked polyvinyl pyrrolidone, corn starch, potato starch, maize starch and modified starches, croscarmellose sodium, crospovidone, sodium starch glycolate, and combinations and mixtures thereof.

The pharmaceutical formulation of the present disclosure may be prepared by methods known to those skilled in the art, such as mixing, dissolving, granulating, dragee-making, levigating, emulsifying, encapsulating, entrapping, or lyophilizing processes. As mentioned above, the zolmitriptan compositions of the present disclosure may include one or more pharmaceutically acceptable carriers such as excipients and adjuvants that facilitate processing of active molecules into preparations for pharmaceutical use.

In some embodiments, the pharmaceutical compositions of the present disclosure are prepared in an oral formulation. For oral administration, the compounds can be formulated readily by combining the active compounds with pharmaceutically acceptable carriers known in the art. Such carriers enable zolmitriptan to be formulated as tablets, pills, dragees, capsules, liquids, gels, syrups, slurries, suspensions and the like, for oral ingestion by a subject. Pharmaceutical compositions for oral use may be obtained as solid excipients, optionally grinding a resulting mixture, and processing the mixture of granules, after adding suitable adjuvants, if desired, to obtain tablets or dragee cores. Such oral pharmaceutical compositions may also be prepared by milling or melt extrusion. Suitable excipients may be any of those disclosed herein and, in particular, fillers such as sugars, including lactose, sucrose, mannitol, or sorbitol; cellulose formulation such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP) formulation. Also, disintegrating agents may be employed, such as cross-linked polyvinylpyrrolidone, agar, or alginic acid or a salt thereof such as sodium alginate. Wetting agents, such as sodium dodecyl sulfate and the like, may be added.

In some embodiments, the zolmitriptan is combined with excipients to form a core comprising zolmitriptan (i.e., an active core). In some embodiments, the active core comprises an inert particle such as a sugar sphere with an appropriate mean particle size. In one embodiment, the inactive core may be a sugar sphere, a cellulose sphere, a spheroidal silicon dioxide bead, a buffer crystal or an encapsulated buffer crystal, such as calcium carbonate, sodium bicarbonate, fumaric acid, tartaric acid, etc. Buffer crystals are useful to alter the microenvironment. Alternatively in accordance with other embodiments, drug-containing microgranules or pellets may be prepared by rotogranulation, high-shear granulation and extrusion-spheronization or compression of the drug (as mini-tablets, e.g., having a diameter of about 2 mm or more), a polymeric binder and optionally fillers/diluents.

In some embodiments, dragee cores may be provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compounds doses.

In some embodiments of the present disclosure, the pharmaceutical composition is prepared for administration by injection (e.g., intravenous, subcutaneous, intramuscular, etc.). In some such embodiments, a pharmaceutical composition comprises a carrier and is formulated in aqueous solution, such as water or physiologically compatible buffers such as Hanks's solution, Ringer's solution, or physiological saline buffer. In some embodiments, other ingredients are included (e.g., ingredients that aid in solubility or serve as preservatives). In some embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as vegetable oils (e.g., sesame oil and the like), synthetic fatty acid esters, such as ethyl oleate or triglycerides. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. Liposomal suspensions containing liposomes that target tissues may be present in suitable formulations.

Parenteral administration of the formulations of the present invention includes intravenous, subcutaneous and intramuscular administrations of the pharmaceutical compositions described herein. Preparations for parenteral administration includes sterile solutions ready for injection, sterile dry soluble products ready to be combined with a solvent just prior to use, including hypodermic tablets, sterile suspensions ready for injection, sterile dry insoluble products ready to be combined with a vehicle just prior to use and sterile emulsions. The solutions can be either aqueous or non-aqueous. Pharmaceutically acceptable carriers used in parenteral preparations include aqueous vehicles, non-aqueous vehicles, antimicrobial agents, isotonic agents, buffers, antioxidants, local anesthetics, suspending and dispersing agents, thickening agents, emulsifying agents, sequestering or chelating agents and other pharmaceutically acceptable substances. Liposome suspensions are also suitable as pharmaceutically acceptable carriers.

In some embodiments, of the present disclosure, the pharmaceutical composition is formulated as a depot preparation. Such depot preparations are typically longer acting than non-depot preparations. In some embodiments, such preparations are administered parenterally (e.g., by injection or implantation). For example, subcutaneous or intramuscular depot injections of a sterile solution containing zolmitriptan is an effective mode of administration. In some embodiments, depot preparations are prepared using suitable polymeric or hydrophobic materials (for example an emulsion in an acceptable oil) or ion exchange resins, or as sparingly soluble derivatives, for example, as a sparingly soluble salt. Thus, for example, zolmitriptan may be formulated with suitable polymeric or hydrophobic materials (e.g., an emulsion in an acceptable oil) or ion exchange resins, or as sparingly soluble derivatives, e.g., a sparingly soluble salt. In some embodiments, the depot preparation provides a 1-day, 7-day or 30-day depot release profile. In some embodiments such preparations are administered on a once-a-day, once-a-week or once-a-month injection schedule.

The concentration of the pharmaceutically active compound can be adjusted so that an injection provides an effective amount to produce the desired pharmacological effect. The exact dose depends on the age, weight and condition of the patient or animal, as is known in the art. In some embodiments, the unit-dose parenteral preparations are packaged in an ampoule or a syringe with a needle. All preparations for parenteral administration must be sterile, as is known and practiced in the art. Illustratively, intravenous or intra-arterial infusion of a sterile aqueous solution containing zolmitriptan is an effective mode of administration.

### Delayed Release Component

In some embodiments, the pharmaceutical compositions described herein comprise one or more immediate release components and one or more delayed release components. In some embodiments, the delayed release component comprises one or more delayed release components (e.g., two, three, four, five, six, seven, eight, nine, and ten or more), each of which may provide a different release profile. In some embodiments, the delayed release portion is a gastrorentive layer. In some embodiments, each delayed release component is formulated to release zolmitriptan in a pulse occurring after a desired delay, the first delayed release component releases zolmitriptan in a pulse and the second delayed release component provides for extended release, or both delayed release components provide for extended release of zolmitriptan. 10951 As used herein, “delayed release” refers to a pharmaceutical formulation that substantially prevents the release (meaning releasing no more than about 5-10%) of the active (e.g., zolmitriptan) contained in the delayed release component for a defined period of time after oral administration. In some embodiments, delayed release substantially prevents release of the active for at least 30 minutes after oral administration, e.g., 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, or more. Delayed release also encompasses formulations in which a portion of the active (e.g., zolmitriptan) is not released from the formulation for a particular period of time.

In some embodiments, the delayed release component can substantially prevent release of at least about 40% (e.g., about 40% to about 60%) of the total amount of zolmitriptan in the pharmaceutical formulation for at least about 30 minutes after oral administration, e.g., 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, or more.

In some embodiments, substantially complete release (e.g., at least about 90-95%) of zolmitriptan from the delayed release component is achieved while the pharmaceutical composition is in the acidic environment of the stomach. Thus, in some embodiments, substantially complete release of zolmitriptan from the delayed release component is achieved before the pharmaceutical composition reaches the relatively less acidic environment of the intestine.

In some embodiments, the delayed release component is appropriately formulated to provide a pulsatile release of zolmitriptan in the acidic environment of the stomach, which occurs at least 30 minutes after oral administration (e.g., about 35, about 40, about 45, about 50, about 55, about 60, about 65, about 70, about 75, about 80, about 85, about 90, about 95, about 100, about 105, about 110, about 115, or about 120 minutes or more).

In some embodiments, the delayed release component is appropriately formulated (e.g., as an extended release formulation) to slowly release zolmitriptan over a period of time, wherein the initial release of zolmitriptan from the delayed release component occurs at least 30 minutes after oral administration and continues for about 30 minutes, about 1 hour, about 1.5 hours, about 2 hours, about 2.5 hours, about 3 hours, about 3.5 hours, about 4 hours, about 4.5 hours, about 5 hours, about 6 hours, about 7 hours, about 8 hours, about 9 hours, about 10 hours or more. In certain other embodiments, the initial release of zolmitriptan from the delayed release component occurs at least 30 minutes after oral administration and occurs for an amount of time in the range of from about 0.5 hour to about 4 hours, e.g., about 1 hour, about 1.5 hour, about 2 hour, about 2.5 hour, about 3 hour, and about 3.5 hours, including of all values and subranges therebetween.

In some embodiments, the delayed release component is appropriately formulated (e.g., as an extended release formulation) to slowly release zolmitriptan over a period of time, wherein the initial release of zolmitriptan from the delayed release component occurs at least 30 minutes after oral administration and continues for at least about 30 minutes, at least about 1 hour, at least about 1.5 hours, at least about 2 hours, at least about 2.5 hours, at least about 3 hours, at least about 3.5 hours, at least about 4 hours, at least about 4.5 hours, at least about 5 hours, at least about 6 hours, at least about 7 hours, at least about 8 hours, at least about 9 hours, or at least about 10 hours or more.

In some embodiments, the delayed release component comprises two or more delayed release components. In some embodiments, a first delayed release component is appropriately formulated to release a first portion of zolmitriptan in the acidic environment of the stomach, and second delayed release component is appropriately formulated to release a second portion of zolmitriptan in the less acidic environment of the lower intestines. The ratio of zolmitriptan released in the stomach compared to the lower intestine may be in the range of from about 99:1 to about 1:99, e.g., about 95:1, about 90:10, about 85:1, about 80:1, about 75:1, about 70:1, about 65:1, about 60:1, about 55:1, about 50:1, about 45:1, about 40:1, about 35:1, about 30:1, about 25:1, about 20:1, about 15:1, about 10:1, and about 5:1, including all values and subranges there between. In some embodiments, the ratio of zolmitriptan that is released in the stomach compared to the lower intestines is in the range of from about 70:30 to about 40:60, e.g., from about 60:40 to about 50:50. The relative amounts of zolmitriptan released in the stomach and in the lower intestines can be selected to reduce the maximum blood plasma levels of zolmitriptan and achieve an AUC that is 80-125% of the AUC to the reference listed product. In some embodiments, delayed release is achieved by appropriately coating a drug-containing component with one or more suitable delayed-release polymers (also referred to as a controlled release polymer or rate-controlling polymer) or embedding the drug in a matrix comprising one or more suitable delayed-release polymers. Suitable delayed-release polymers include pharmaceutically acceptable water-insoluble polymers (also referred to as hydrophobic polymers), pharmaceutically acceptable water-soluble polymers (also referred to as hydrophilic polymers), pharmaceutically acceptable gastrosoluble polymers, pharmaceutically acceptable enteric polymers, and combinations thereof.

In some embodiments, non-limiting examples of pharmaceutically acceptable water-soluble polymers include homopolymers and copolymers of N-vinyl lactams, including homopolymers and copolymers of N-vinyl pyrrolidone, e.g. polyvinylpyrrolidone (PVP), copolymers of N-vinyl pyrrolidone and vinyl acetate or vinyl propionate, cellulose esters and cellulose ethers, in particular methylcellulose and ethylcellulose, hydroxyalkylcelluloses, in particular hydroxypropylcellulose, hydroxyalkylalkylcelluloses, and hydroxypropylmethylcellulose, cellulose phthalates, succinates, butyrates, or trimellitates, in particular cellulose acetate phthalate, hydroxypropylmethylcellulose phthalate, hydroxypropylmethylcellulose succinate, and hydroxypropylmethylcellulose acetate succinate; high molecular polyalkylene oxides such as polyethylene oxide and polypropylene oxide and copolymers of ethylene oxide and propylene oxide, polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates), polyacrylamides, vinyl acetate polymers such as copolymers of vinyl acetate and crotonic acid, partially hydrolyzed polyvinyl acetate (also referred to as partially saponified “polyvinyl alcohol”), polyvinyl alcohol, polyethylene glycol oligo- and polysaccharides such as carrageenans, galactomannans and xanthan gum, or mixtures of one or more thereof.

Non-limiting examples of gastrosoluble polymers include maltrin, an aminoalkyl methacrylate copolymer available under the trade name of EUDRAGIT® (type E100 or EPO), polyvinylacetal diethylaminoacetate e.g., AEA® available from Sankyo Company Limited, Tokyo (Japan), and the like.

Non-limiting examples of enteric polymers include cellulose acetate phthalate (CAP), cellulose acetate succinate, methylcellulose phthalate, hydroxymethylethylcellulose phthalate, hydroxypropylmethylcellulose phthalate (HPMCP), hydroxypropylmethylcellulose acetate succinate (HPMCAS), polyvinyl alcohol phthalate, polyvinyl butyrate phthalate, polyvinyl acetal phthalate (PVAP), a copolymer of vinyl acetate/maleic anhydride, a copolymer of vinylbutylether/maleic anhydride, a copolymer of styrene/maleic acid monoester, a copolymer of methyl acrylate/methacrylic acid, a copolymer of styrene/acrylic acid, a copolymer of methyl acrylate/methacrylic acid/octyl acrylate, a copolymer of methacrylic acid/methyl methacrylate, cellulose acetate hexahydrophthalate, hydroxypropyl methylcellulose hexahydrophthalate, hydroxypropyl methylcellulose phthalate, cellulose propionate phthalate, cellulose acetate maleate, cellulose acetate trimellitate, cellulose acetate butyrate, cellulose acetate propionate, methacrylic acid/methacrylate polymer (acid number 300 to 330 and also known as EUDRAGIT L), methacrylic acid-methyl methacrylate copolymer, ethyl methacrylate-methylmethacrylate-chlorotrimethylammonium ethyl methacrylate copolymer, and the like, and combinations comprising one or more of the foregoing enteric polymers. Other examples include natural resins, such as shellac, Sandarac, copal collophorium, and combinations comprising one or more of the foregoing polymers. Yet other examples of enteric polymers include synthetic resin bearing carboxyl groups. The term “enteric polymer” as used herein is defined to mean a polymeric substance that when used in an enteric coat formulation, is substantially insoluble and/or substantially stable under acidic conditions at a pH of less than about 5 and which are substantially soluble or can decompose under conditions exhibiting a pH of about 5 or more.

Non-limiting examples of hydrophilic polymers include hydroxypropyl celluloses (HPC), hydroxypropyl methylcelluloses, methylcelluloses, polyethylene oxides, sodium carboxymethyl celluloses, and the like, or combinations thereof.

In some embodiments, the delayed release coating comprises about 40 wt % to about 95 wt % of any of pharmaceutically acceptable polymers listed above (e.g., about 45%, about 50%, about 55%, about 60%, about 65%, about 70%, about 75%, about 80%, about 85%, about 90%, including all values and subranges therebetween) and about 5 wt % to about 60 wt % plasticizer (e.g., about 10%, about 15%, about 20%, about 25%, about 30%, about 35%, about 40%, about 45%, about 50%, about 55%, including all values and subranges therebetween) based on the total weight of the polymer coating. The relative proportions of ingredients, notably the ratio of the enteric polymer to plasticizer can be varied according to methods known to those of skill in the art of pharmaceutical formulation.

### Extended Release Component

In some embodiments, the pharmaceutical compositions described herein comprise one or more immediate release components and one or more extended release components, where the extended release components release the zolmitriptan over an extended period of time. An extended period of time is longer than that of the immediate release component as described herein, such that the total amount of active agent in an extended release component is released over a period of about 1 to 24 hours, e.g., over about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours, including all ranges therebetween. When combined in a pharmaceutical composition, the combination of immediate release and extended release components in the pharmaceutical composition release less than about 75% of the total dose of zolmitriptan within about 30 minutes, e.g., as measured using the dissolutions tests described herein. In some embodiments, such pharmaceutical compositions release about 74%, about 73%, about 72%, about 71%, about 70%, about 69%, about 68%, about 67%, about 66%, about 65%, about 64%, about 63%, about 62%, about 61%, about 60%, about 55%, about 50%, about 45%, about 40%, about 35%, about 30%, about 25%, about 20%, about 15%, about 10%, and about 5% or less, of the total dose of zolmitriptan within about 30 minutes, e.g., as measured using the dissolutions tests described herein. Advantageously, such pharmaceutical compositions achieve equivalent therapeutic efficacy with decreased maximum exposure levels compared to a reference listed product (i.e., ZOMIG) as described herein.

In some embodiments, the extended release component comprises one or more extended release components (e.g., two, three, four, five, six, seven, eight, nine, and ten or more), each of which may provide a different release profile. In some embodiments, the extended release portion is a gastroretentive layer.

In some embodiments, the extended release component is prepared by embedding the drug in a matrix comprising one or more suitable extended-release polymers (such as water-swellable polymer for a gastroretentive formulation).

In some embodiments, the extended-release component is a gastro-retentive composition. Such compositions have prolonged residence time in the stomach, which provides for delayed gastric emptying of the zolmitriptan. Examples include floating drug delivery systems, also known as hydrodynamically balanced systems, swelling and expanding systems, modified-shape systems, high-density systems, and other delayed gastric emptying systems (U.S. Pat. No. 9,000,046, herein incorporated by reference in its entirety). Gastro-retentive compositions may also be formulated with a bioadhesive (e.g., muco-adhesive), a particle diameter which substantially prevents drug-containing particles from passing through the stomach to the lower intestines, and floating drug delivery systems. In some embodiments, the bioadhesive is selected from the group consisting of polycarbophil, lectins, carbopol, chitosan, carboxymethylcellulose (CMC), pectin, gliadin, polyethylene glycol, tragacanth, sodium alginate, cholestyramine, polyacrylic acid, and sucralfate (See Madal, U. K., et al. “Gastro-retentive drug delivery systems and their in vivo success: A recent update” Asian J. of Pharm. Sci. 2016, 11, 575-584; and Sharma, A. R.; et al. “Gastroretentive Drug Delivery System: An Approach to Enhance Gastric Retention for Prolonged Drug Release” International J. of Pharm. Sci. and Res. 2014, 5, 1095-1106, each of which is herein incorporated by reference in its entirety).

In some embodiments, the extended release component is a gastrorentive composition having zolmitriptan in water swellable polymer matrix. Water-swellable polymers suitable for use herein are those that swell in a dimensionally unrestrained manner upon contact with water. Such polymers may also gradually erode over time. Examples of such polymers include polyalkylene oxides, such as polyethylene glycols, particularly high molecular weight polyethylene glycols; cellulose polymers and their derivatives including, but not limited to, hydroxyalkyl celluloses, hydroxymethyl cellulose, hydroxyethyl cellulose, hydroxypropyl cellulose, hydroxypropylmethyl cellulose, carboxymethylcellulose, microcrystalline cellulose; polysaccharides and their derivatives; chitosan; poly(vinyl alcohol); xanthan gum; maleic anhydride copolymers; poly(vinyl pyrrolidone); starch and starch-based polymers; maltodextrins; poly (2-ethyl-2-oxazoline); poly(ethyleneimine); polyurethane; hydrogels; crosslinked polyacrylic acids; and combinations or blends of any of the foregoing.

Further examples are copolymers, including block copolymers and graft polymers. Specific examples of copolymers are PLURONIC® and TECTONIC®, which are polyethylene oxide-polypropylene oxide block copolymers available from BASF Corporation, Chemicals Div., Wyandotte, Mich., USA. Further examples are hydrolyzed starch polyacrylonitrile graft copolymers, commonly known as “Super Slurper” and available from Illinois Corn Growers Association, Bloomington, Ill., USA.

In some embodiments, swellable, erodible hydrophilic polymers suitable for forming the gastric retentive portion of the dosage forms described herein are poly(ethylene oxide), hydroxypropyl methyl cellulose, and combinations of poly(ethylene oxide) and hydroxypropyl methyl cellulose. Poly(ethylene oxide) is used herein to refer to a linear polymer of unsubstituted ethylene oxide. The molecular weight of the poly(ethylene oxide) polymers can range from about 9×105 Daltons to about 8×106 Daltons. Exemplary molecular weight poly(ethylene oxide) polymers include about 9×105 Daltons (e.g., SENTRY™ POLYOX™ WSR 1105). In some embodiments, the rate of wetting, swelling and erosion can be modified by changing the molecular weight Polyox™.

In some embodiments, the water-swellable polymer is selected from the group consisting of polyalkylene oxides, cellulose polymers and derivatives thereof, polysaccharides and derivatives thereof, chitosan, poly(vinyl alcohol), xanthan gum, maleic anhydride copolymers, poly(vinyl pyrrolidone), starch and starch-based polymers; maltodextrin, poly (2-ethyl-2-oxazoline), poly(ethyleneimine), polyurethane, hydrogels, crosslinked polyacrylic acids, and combinations thereof.

In some embodiments, the water-swellable polymer is selected from the group consisting of high molecular weight polyethylene oxide, hydroxyalkyl cellulose, hydroxymethyl cellulose, hydroxyethyl cellulose, hydroxypropyl cellulose, hydroxypropylmethyl cellulose, carboxymethylcellulose, and microcrystalline cellulose.

In some embodiments, the water swellable polymer is polyethylene oxide.

In some embodiments, the composition comprises about 15% to about 75% by weight of a water-swellable polymer, including about 15%, about 20%, about 25%, about 30%, about 35%, about 40%, about 45%, about 50%, about 55%, about 60%, about 65%, about 70%, to about 75%, including all values and subranges therebetween. In some embodiments, the composition comprises about 35-55% by weight of a water-swellable polymer. In some embodiments, the water-swellable polymer is polyethylene oxide. In some embodiments, the composition comprises about 35-55% by weight of polyethylene oxide. In some embodiments, extended release is achieved by appropriately coating a drug-containing component with one or more suitable extended-release polymers (also referred to as a rate-controlling polymer). Suitable extended-release polymers include, for example, pharmaceutically acceptable water-insoluble polymers (also referred to as hydrophobic polymers), and pharmaceutically acceptable water-swellable polymers.

In some embodiments, the extended release portion comprises drug-containing particles coated with an extended release coating. In some embodiments, multiparticulate formulation.

Non-limiting examples of pharmaceutically acceptable water-insoluble polymers include acrylic polymers, methacrylic acid polymers, acrylic copolymers, such as a methacrylic acid-ethyl acrylate copolymer available under the trade name of EUDRAGIT® (type L, RL, RS and NE30D), and their respective esters, zein, waxes, shellac and hydrogenated vegetable oil, cellulose derivatives, such as ethyl cellulose, cellulose acetate, cellulose acetate butyrate, and the like.

In some embodiments, the extended release coating comprises about 40 wt % to about 95 wt % of any of pharmaceutically acceptable polymers listed above (e.g., about 45%, about 50%, about 55%, about 60%, about 65%, about 70%, about 75%, about 80%, about 85%, about 90%, including all values and subranges therebetween) and about 5 wt % to about 60 wt % plasticizer (e.g., about 10%, about 15%, about 20%, about 25%, about 30%, about 35%, about 40%, about 45%, about 50%, about 55%, including all values and subranges therebetween) based on the total weight of the polymer coating.

### Osmotic Release Systems

In some embodiments, the delayed release is an osmotic system. An osmotic system is a core with a semi-permeable outer membrane and one or more openings. The semipermeable membrane is impermeable to zolmitriptan, but permits entry of water by osmosis from the outside into the osmotic system. As the core passes through the body, water is absorbed through the semipermeable membrane via osmosis, and the resulting osmotic pressure is used to push the active drug through the opening(s) in the core. Total zolmitriptan release and the release rate can be controlled by appropriately selecting the thickness and porosity of the semipermeable membrane, the composition of the core and the number and size of the opening(s). Formulation aspects, administration forms and information about preparation processes are described, for example, in the following publications:
Santus, G., Baker, R. W., “Osmotic drug delivery: a review of the patent literature”, Journal of Controlled Release 35 (1995), 1-21;
Verma, R. K., Mishra, B., Garg, S., “Osmotically controlled oral drug delivery”, Drug Development and Industrial Pharmacy 26 (2000), 695-708;
Verma, R. K., Krishna, D. M., Garg, S., “Formulation aspects in the development of osmotically controlled oral drug delivery systems”, Journal of Controlled Release 79 (2002), 7-27;
Verma, R. K., Arora, S., Garg, S., “Osmotic Pumps in drug delivery”, Critical Reviews in Therapeutic Drug Carrier Systems 21 (2004), 477-520
Malaterre, V., Ogorka, J., Loggia, N., Gurny, R., “Oral osmotically driven systems: 30 years of development and clinical use”, European Journal of Pharmaceutics and Biopharmaceutics 73 (2009), 311-323; and
U.S. Pat. Nos. 4,327,725, 4,765,989;

each of which are herein incorporated by reference in its entirety for all purposes.

Both single-chamber systems (elementary osmotic pump) and two-chamber systems (push-pull systems) are suitable for delayed release component described here.

In some embodiments, the shell of the osmotic release system comprises either a single-chamber system or a two-chamber system of the semipermeable membrane. Non-limiting examples of shell materials include cellulose acetate or mixtures of cellulose acetate and polyethylene glycol.

In some embodiments, a coating, for example a photoprotective and/or colored coating, can be applied to the shell. Materials suitable for this purpose are, for example, polymers such as polyvinyl alcohol, hydroxypropylcellulose and/or hydroxypropylmethylcellulose, where appropriate in combination with suitable plasticizers such as, for example, polyethylene glycol or polypropylene glycol and pigments such as, for example, titanium dioxide or iron oxides.

In some embodiments, the core in the osmotic single-chamber system comprises: (i) 2 to 30 wt % zolmitriptan; (ii) 20 to 50 wt % xanthan, and (iii) 10 to 30 wt % of a vinylpyrrolidone-vinyl acetate copolymer, where the difference from 100% is formed where appropriate by one or more additional ingredients selected from the group of further hydrophilic, swellable polymers, osmotically active additives and pharmaceutically acceptable excipients.

In some embodiments, the core of an osmotic single-chamber system comprises a hydrophilic water-swellable polymer, e.g., xanthan. Xanthan is an anionic heteropolysaccharide that is commercially available for example under the name Rhodigel® (produced by Rhodia). In some embodiments, the hydrophilic water-swellable polymer is present in an amount of from about 20 to about 50% (e.g., about 25%, about 30%, about 35%, about 40%, about 45%, including all values and subranges therebetween) based on the total mass of the core ingredients.

Another ingredient of the core may be a vinylpyrrolidone-vinyl acetate copolymer. This copolymer is known in the art and can be produced with any desired monomer mixing ratios. A non-limiting example of such a copolymer is Kollidon® VA64 (produced by BASF). It generally has a weight average molecular weight (Mw), determined by light-scattering measurements, of about 45 000 to about 70 000. In some embodiments, the amount of the vinylpyrrolidone-vinyl acetate copolymer in the core is from about 10% to about 30% (e.g., about 15%, about 20%, about 25%, including all values and subranges therebetween), based on the total mass of the core ingredients.

In some embodiments, the hydrophilic swellable polymers are present in the core. Non-limiting examples of hydrophilic swellable polymers include, for example, hydroxypropylcellulose, hydroxypropylmethylcellulose, sodium carboxymethylcellulose, sodium carboxymethyl starch, polyacrylic acids and salts thereof.

In some embodiments, the osmotic release system includes a water swellable substance. Suitable water swellable substances include compounds that are able to expand when they are exposed to an aqueous solution, such as gastro-intestinal fluid. One or more water swellable substances may be present in the core material together. Alternatively, one or more water swellable substances may be included in a swelling layer applied onto the core material.

Suitable water swellable substances include, for example, low-substituted hydroxypropyl cellulose, e.g., L-HPC; cross-linked polyvinyl pyrrolidone (PVP-XL), e.g., Kollidon® CL and Poly-Plasdone® XL; cross-linked sodium carboxymethylcellulose, e.g., Ac-di-sol, Primellose®; sodium starch glycolate, e.g., Primojel®; sodium carboxymethylcellulose, e.g., Nym-cel ZSBlO®; sodium carboxymethyl starch, e.g., Explotab®; ion-exchange resins, e.g., Dowex® or Amber-Lite®; microcrystalline cellulose, e.g., Avicel®; starches and pregelatinized starch, e.g., Starch 1500®, Sepistab ST200®; formalin-casein, e.g., Plas-Vita®, and mixtures thereof.

In some embodiments, the osmotic pump compositions comprise one or more osmotically active additives in the core, for example, pharmaceutically acceptable water-soluble substances, such as, the water-soluble excipients described in pharmacopoeias or “Remington Pharmaceutical Science,” (1985) 17th ed. Edited by Alfonso R. Gennaro. Mack Publishing Co., Easton, PA., which is herein incorporated by reference in its entirety for all purposes. In some embodiments, the osmotically active additive is selected from water-soluble salts of inorganic or organic acids or nonionic organic substances with high solubility in water, such as, for example, carbohydrates, especially sugars, sugar alcohols or amino acids. In some embodiments, the osmotically active additive is selected from inorganic salts such as chlorides, sulfates, carbonates and bicarbonates of alkali metals or alkaline earth metals, such as lithium, sodium, potassium, magnesium, calcium, and phosphates, hydrogen phosphates or dihydrogen phosphates, acetates, succinates, benzoates, citrates or ascorbates thereof. In some embodiments, the osmotically active additive is selected from pentoses such as arabinose, ribose or xylose, hexoses such as glucose, fructose, galactose or mannose, disaccharides such as sucrose, maltose or lactose or trisaccharides such as raffinose. The water-soluble amino acids include glycine, leucine, alanine or methionine. Sodium chloride is particularly preferably used according to the invention. In some embodiments, the osmotically active additives are present in an amount of from 10 to 30% based on the total mass of the core ingredients.

In some embodiments, the core of the osmotic pump compositions core comprise buffer substances (such as sodium bicarbonate), binders (such as hydroxypropylcellulose), hydroxypropylmethylcellulose and/or polyvinylpyrrolidone, lubricants (such as magnesium stearate), wetting agents (such as sodium lauryl sulfate) and/or flow regulators (such as colloidal silicon dioxide).

The present disclosure further relates to a process for producing an osmotic single-chamber system, where the components of the core are mixed together, subjected where appropriate to wet or dry granulation, and subsequently tableted, and the core produced in this way is coated with the shell that is then covered, where appropriate, with a photoprotective and/or colored coating, and which is provided with one or more orifices.

In the osmotic two-chamber system, the core consists of two layers, one active ingredient layer and one osmotic layer. An osmotic two-chamber system of this type is described in, for example, U.S. Pat. No. 4,612,008, the disclosure of which is incorporated herein by reference.

The osmotically active additives used in the core of the osmotic two-chamber system may be the same as in the case of the single-chamber system described above.

The pharmaceutically acceptable excipients used in the core of the osmotic two-chamber system may be the same as in the case of the single-chamber system described above. In some embodiments, binders (such as hydroxypropylcellulose), hydroxypropylmethylcellulose and/or polyvinylpyrrolidone, lubricants (such as magnesium stearate), wetting agents (such as sodium lauryl sulfate) and/or flow regulators (such as colloidal silicon dioxide), and a coloring pigment (such as iron oxide) in one of the two layers is used to differentiate active ingredient layer and osmosis layer.

### Matrix

In some embodiments, the delayed release component is a matrix. As used herein, the term “matrix” means a composition in which the drug is embedded or dispersed in water soluble, water insoluble, or hydrophilic polymers, or lipophilic materials, in order to achieve delayed release of the drug. The mechanisms of the drug release generally involve drug diffusion through a viscous gel layer or tortuous channels; and/or drug dissolution via gradual erosion or degradation of the polymer(s). In some embodiments, the matrix comprises swellable/erodable polymers, for example hydrophilic polymers that in contact with the water form a high viscosity gel. In some embodiments, the matrix comprises water-insoluble polymers or lipophilic polymers.

In some embodiments, the matrix is prepared using one or more hydrophilic polymers (e.g., hydroxypropyl cellulose, hydroxypropyl methylcellulose, polyethylene oxide), one or more lipophilic materials (e.g., carnauba wax, hardened castor oil, hardened rape seed oil, polyglycerin fatty acid ester), and/or coating tablets or granules with one or more delayed release polymers (e g., cellulose polymers such as ethylcellulose; acrylic acid copolymer such as aminoalkyl methacrylate copolymer RS [Eudragit RS (trade name, Degussa Co.)], ethyl acrylate-methyl methacrylate copolymer suspension [Eudragit NE (trade name, Degussa Co.)]).

In some embodiments, the delayed release component is a matrix comprising one or more hydrophilic polymers. Herein, the hydrophilic polymer means a polymer that can control the release of the zolmitriptan by absorbing water to become hydrogel and allow the zolmitriptan contained in the matrix to diffuse out of the same.

In some embodiments, the viscosity of the hydrophilic polymer is, for example, about 1 mPa·s to about 200000 mPa·s, or about 4 mPa·s to about 120000 mPa·s, or about 4 mPa·s to about 5000 mPa·s as measured using a Brookfield viscometer in a 2% by weight aqueous solution at 20° C. The release duration of the zolmitriptan from the matrix can be modified by appropriately selecting the viscosity of the hydrophilic polymer.

Non-limiting examples of suitable hydrophilic polymers include hydroxypropyl celluloses (HPC) such as HPC-SSL (trade name, manufactured by NIPPON SODA CO., viscosity of 2% by weight aqueous solution at 20° C.: 2.0-2.9 mPa·s), HPC-SL (trade name, manufactured by NIPPON SODA CO., viscosity of 2% by weight aqueous solution at 20° C.: 3.0-5.9 mPa·s), HPC-L (trade name, manufactured by NIPPON SODA CO., viscosity of 2% by weight aqueous solution at 20° C.: 6.0-10.0 mPa·s), HPC-M (trade name, manufactured by NIPPON SODA CO., viscosity of 2% by weight aqueous solution at 20° C.: 150-400 mPa·s), HPC-H (trade name, manufactured by NIPPON SODA CO., viscosity of 2% by weight aqueous solution at 20° C.: 1000-4000 mPa·s); hydroxypropyl methylcelluloses such as Metolose SB-4 (trade name, manufactured by Shin-Etsu Chemical CO., viscosity of 2% by weight aqueous solution at 20° C.: about 4 mPa·s), TC-5RW (trade name, manufactured by Shin-Etsu Chemical CO., viscosity of 2% by weight aqueous solution at 20° C.: about 6 mPa·s), TC-5S (trade name, manufactured by Shin-Etsu Chemical CO., viscosity of 2% by weight aqueous solution at 20° C.: about 15 mPa·s), Metolose 60SH-50 (trade name, manufactured by Shin-Etsu Chemical CO., viscosity of 2% by weight aqueous solution at 20° C.: about 50 mPa·s), Metolose 65SH-50 (trade name, manufactured by Shin-Etsu Chemical CO., viscosity of 2% by weight aqueous solution at 20° C.: about 50 mPa·s), Metolose 90SH-100 (trade name, manufactured by Shin-Etsu Chemical CO., viscosity of 2% by weight aqueous solution at 20° C.: about 100 mPa·s), Metolose 90SH-100SR (trade name, manufactured by Shin-Etsu Chemical CO., viscosity of 2% by weight aqueous solution at 20° C.: about 100 mPa·s), Metolose 65SH-400 (trade name, manufactured by Shin-Etsu Chemical CO., viscosity of 2% by weight aqueous solution at 20° C.: about 400 mPa·s), Metolose 90SH-400 (trade name, manufactured by Shin-Etsu Chemical CO., viscosity of 2% by weight aqueous solution at 20° C.: about 400 mPa·s), Metolose 65SH-1500 (trade name, manufactured by Shin-Etsu Chemical CO., viscosity of 2% by weight aqueous solution at 20° C.: about 1500 mPa·s), Metolose 60SH-4000 (trade name, manufactured by Shin-Etsu Chemical CO., viscosity of 2% by weight aqueous solution at 20° C.: about 4000 mPa·s), Metolose 65SH-4000 (trade name, manufactured by Shin-Etsu Chemical CO., viscosity of 2% by weight aqueous solution at 20° C.: about 4000 mPa·s), Metolose 90SH-4000 (trade name, manufactured by Shin-Etsu Chemical CO., viscosity of 2% by weight aqueous solution at 20° C.: about 4000 mPa·s), Metolose 90SH-4000SR (trade name, manufactured by Shin-Etsu Chemical CO., viscosity of 2% by weight aqueous solution at 20° C.: about 4000 mPa·s), Metolose 90SH-30000 (trade name, manufactured by Shin-Etsu Chemical CO., viscosity of 2% by weight aqueous solution at 20° C.: about 30000 mPa·s), Metolose 90SH-100000 (trade name, manufactured by Shin-Etsu Chemical CO., viscosity of 2% by weight aqueous solution at 20° C.: about 100000 mPa·s), Metolose 90SH-100000SR (trade name, manufactured by Shin-Etsu Chemical CO., viscosity of 2% by weight aqueous solution at 20° C.: about 100000 mPa·s); methylcelluloses such as Metolose SM15 (trade name, manufactured by Shin-Etsu Chemical CO.; viscosity: about 15 mPa·s, 2% by weight aqueous solution, 20° C.), Metolose SM25 (trade name, manufactured by Shin-Etsu Chemical CO., viscosity of 2% by weight aqueous solution at 20° C.: about 25 mPa·s), Metolose SM100 (trade name, manufactured by Shin-Etsu Chemical CO., viscosity of 2% by weight aqueous solution at 20° C.: about 100 mPa·s), Metolose SM400 (trade name, manufactured by Shin-Etsu Chemical CO., viscosity of 2% by weight aqueous solution at 20° C.: about 400 mPa·s), Metolose SM1500 (trade name, manufactured by Shin-Etsu Chemical CO., viscosity of 2% by weight aqueous solution at 20° C.: about 1500 mPa·s), Metolose SM4000 (trade name, manufactured by Shin-Etsu Chemical CO., viscosity of 2% by weight aqueous solution at 20° C.: about 4000 mPa·s), Metolose SM8000 (trade name, manufactured by Shin-Etsu Chemical CO., viscosity of 2% by weight aqueous solution at 20° C.: about 8000 mPa·s); polyethylene oxides such as WSR N-12K (trade name, manufactured by Union Carbide Co., viscosity of 2% by weight aqueous solution at 20° C.: 400-800 mPa·s), WSR N-60K (trade name, manufactured by Union Carbide Co., viscosity of 2% by weight aqueous solution at 20° C.: 2000-4000 mPa·s), WSR 301 (trade name, manufactured by Union Carbide Co., viscosity of 1% by weight aqueous solution at 25° C.: 1500-4500 mPa·s), WSR Coagulant (trade name, manufactured by Union Carbide Co., viscosity of 1% by weight aqueous solution at 25° C.: 4500-7500 mPa·s), WSR 303 (trade name, manufactured by Union Carbide Co., viscosity of 1% by weight aqueous solution at 25° C.: 7500-10000 mPa·s), WSR 308 (trade name, manufactured by Union Carbide Co., viscosity of 1% by weight aqueous solution at 25° C.: 10000-15000 mPa·s); sodium carboxymethyl celluloses such as Sunrose F-150MC (trade name, manufactured by Nippon Paper Industries Co., viscosity of 1% by weight aqueous solution at 25° C.: 1200-1800 mPa·s), Sunrose F-300MC (trade name, manufactured by Nippon Paper Industries Co., viscosity of 1% by weight aqueous solution at 25° C.: 2500-3000 mPa·s), Sunrose F-1000MC (trade name, manufactured by Nippon Paper Industries Co., viscosity of 1% by weight aqueous solution at 25° C.: 8000-12000 mPa·s); and the like, or combinations thereof.

In some embodiments, the hydrophilic matrix further comprises a pH-dependent polymer. The release duration of the zolmitriptan from the matrix may be modified by appropriately selecting the amount of the pH-dependent polymer.

The term “pH-dependent” refers to a polymer that releases the zolmitriptan at a certain pH. Non-limiting examples of suitable pH-dependent polymers include hydroxypropyl methylcellulose phthalate, cellulose acetate phthalate, carboxymethyl ethyl cellulose, methyl methacrylate-methacrylic acid copolymer, methacrylic acid-ethyl acrylate copolymer, ethyl acrylate-methyl methacrylate-trimethylammoniumethyl methacrylate chloride copolymer, methyl methacrylate-ethyl acrylate copolymer, methacrylic acid-methyl acrylate-methyl methacrylate copolymer, hydroxypropyl cellulose acetate succinate, polyvinyl acetate phthalate and the like, and combinations thereof.

Pharmaceutically acceptable carriers suitable for matrix formulations include various organic or inorganic carrier substances, for example, excipients, lubricants, binders, disintegrants and the like. Further, pharmaceutically acceptable additives such as antioxidants, colorants, sweeteners and the like can be used.

Non-limiting examples of suitable excipients include lactose, D-mannitol, D-sorbitol, starch, a starch, dextrin, crystalline cellulose, low substituted hydroxypropyl cellulose, carboxymethyl cellulose sodium, gum arabic, dextrin, pullulan, light silicic acid anhydride, synthetic aluminum silicate, magnesium aluminometasilicate, and the like.

Non-limiting examples of suitable lubricants include magnesium stearate, calcium stearate, talc, colloidal silica and the like.

Non-limiting examples of suitable binders include a starch, sucrose, gelatin, gum arabic, methylcellulose, carboxymethyl cellulose, carboxymethyl cellulose sodium, crystalline cellulose, white sugar, D-mannitol, trehalose, dextrin, pullulan, hydroxypropyl cellulose, hydroxypropyl methylcellulose, polyvinyl pyrrolidone, and the like.

Non-limiting examples of suitable disintegrants include lactose, white sugar, starch, carboxymethyl cellulose, carboxymethyl cellulose calcium, croscarmellose sodium, carboxymethylstarch sodium, light silicic acid anhydride, low substituted hydroxypropyl cellulose, and the like.

Non-limiting examples of suitable antiseptics include p-hydroxybenzoic esters, chlorobutanol, benzyl alcohol, phenethyl alcohol, dehydroacetic acid, sorbic acid and the like.

Non-limiting examples of suitable antioxidants include bisulfate, ascorbate, and the like.

Non-limiting examples of suitable colorants include water-soluble edible tar pigments (e.g., edible pigments such as edible Red No. 2 and No. 3, edible Yellow No. 4 and No. 5, edible Blue no. 1 and No. 2, and the like), water-insoluble lake pigments (e.g., aluminum salt of the above-described water-soluble edible tar pigments), natural pigments (e.g., p-carotene, chlorophyll, colcothar, yellow iron sesquioxide), and the like.

Non-limiting examples of suitable sweeteners include saccharin sodium, glycyrrhizin dipotassium, aspartame, stevia, and the like.

### Delayed Release Tablet

In some embodiments, the delayed release component is a delayed-release tablet comprising a core, and one or more coatings. In some embodiments, the core comprises zolmitriptan, and any of the excipients described herein, such a lubricant, and a binder and/or a filler, and a glidant as well as other excipients. The one or more coatings may be, for example, a semi-permeable coating to achieve delayed or extended release of the drug. In some embodiments, the coating comprises a water-insoluble polymer, a plasticizer and a water-soluble polymer (such as those described herein). In some embodiments, the water-insoluble polymer is selected from cellulose ether (such as ethylcellulose), a cellulose ester (such as cellulose acetate), polyvinylalcohol, and the like. Other excipients can optionally also be present in the coating, as for example acrylic acid derivatives (such and EUDRAGIT), pigments, etc. If multiple coatings are used, they may be the same or different.

The relative proportions of ingredients, notably the ratio of water-insoluble polymer to water-soluble polymer, can be varied depending on the release profile to be obtained (where a more delayed release is generally obtained with a higher amount of water-insoluble polymer). In some embodiments, the ratio of water-insoluble polymer to water-soluble polymer is in the range of from about 99:1 to about 1:1, e.g., about 95:1, about 90:10, about 85:1, about 80:1, about 75:1, about 70:1, about 65:1, about 60:1, about 55:1, about 50:1, about 45:1, about 40:1, about 35:1, about 30:1, about 25:1, about 20:1, about 15:1, about 10:1, and about 5:1, including all values and subranges therebetween.

### Mini-Tablets

In some embodiments, the delayed release component is formulated to have a dosage unit size sufficient to remain in the stomach and avoid passing to the lower intestines for an appropriate period of time to allow the zolmitriptan to be released in the acidic environment of the stomach.

In some embodiments, the pharmaceutical composition comprises a plurality of mini-tablets (also known as “mini-tabs”), said mini-tablets having a diameter of less than or equal to about 5 mm. In other embodiments, the mini-tablets comprise zolmitriptan within a matrix of polymer(s), or the mini-tablets optionally coated with a delayed release polymer. In some embodiments, the matrix polymer is one or combination of hydrophilic polymers described herein. In some embodiments, the pharmaceutical composition comprises a plurality of enteric coated mini-tablets.

In some embodiments, the mini-tablet further comprises a filler, a lubricant, and/or a glidant. For example, in some embodiments, the mini-tablet comprises from 5-50% of zolmitriptan, from 20-50% of a matrix polymer, from 20-50% of a filler, from 0.1-5% of a lubricant, and from 0.1-5% of a glidant, based on total weight of the composition. In some embodiments, the matrix polymer is hypromellose (also known as hydroxypropyl methylcellulose or “HPMC”), the filler is microcrystalline cellulose, the lubricant is magnesium stearate, and the glidant is colloidal silicon dioxide.

In some embodiments, the pharmaceutical compositions disclosed herein comprise a plurality of mini-tablets, for example in the range of from 2 to 30 mini-tablets, e.g., 3, 4, 5, 6, 7, 8, 9, 10, 11, 12, 13, 14, 15, 16, 17, 18, 19, 20, 21, 22, 23, 24, 25, 26, 27, 28, and 29 mini-tablets, inclusive of all values and subranges therebetween. In some embodiments, the mini-tablets are contained in a capsule or sachet for oral administration. In some embodiments, the capsule is a hard gelatin or hydroxypropylmethylcellulose (HPMC) capsule. In some embodiments, the capsule contains a particulate overfill, such as microcrystalline cellulose. In some embodiments, the capsule comprises from 2 to 30 mini-tablets, e.g., 3, 4, 5, 6, 7, 8, 9, 10, 11, 12, 13, 14, 15, 16, 17, 18, 19, 20, 21, 22, 23, 24, 25, 26, 27, 28, and 29 mini-tablets, including all values and subranges therebetween.

In some embodiments, the mini-tablets have a diameter of less than or equal to about 5 mm, e.g., about 4.5 mm or less, about 4 mm or less, about 3.5 mm or less, about 3.0 mm or less, about 2.5 mm or less, or about 2 mm. In some embodiments, the mini-tablets have a diameter in the range of from about 2 mm to about 5 mm, including, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, about 4.0 mm, about 4.1 mm, about 4.2 mm, about 4.3 mm, about 4.4 mm, about 4.5 mm, about 4.6 mm, about 4.7 mm, about 4.8 mm, and about 4.9 mm, including all values and subranges therebetween. The mini-tablets may have any shape convenient to the skilled person e.g. spherical or cylindrical. In one embodiment, the mini-tablets are round and convex (known in the art as “round standard convex”).

In some embodiments, the mini-tablets are formulated to modify the release of the zolmitriptan, for example, by coating the mini-tablets with a polymer disclosed herein. In other embodiments, the zolmitriptan is embedded or dispersed in the matrix polymer which is formulated as a mini-tablet. In some embodiments, mini-tablets further comprise a filler, a lubricant, or a glidant (one or more such components may be utilized), or combinations thereof.

Suitable matrix polymers include hydrophilic water soluble polymers, for example any of the hydrophilic polymers listed herein, including hydroxypropyl celluloses, hydroxypropyl methylcelluloses, polyethylene oxides, sodium carboxymethyl celluloses, In some embodiments, the hydrophilic water soluble polymer is a high molecular weight polymers (i.e. 100,000 to 800,000 daltons), such as hydroxypropyl methylcellulose polymers (aka HPMC or hypromellose) which is marketed under tradenames such as Methocel™, for example Methocel™ K100M, Methocel™ K15M, or Methocel™ K4M, suitably Methocel™ K15M. In some embodiments, the mini-tablets containing a matrix comprise of from about 20 to about 60 wt % of a matrix polymer, e.g., about 25%, about 30%, about 35%, about 40%, about 45%, about 50%, or about 55%, including all values and subranges therebetween.

Suitable fillers for mini-tablets include any filler listed herein, including microcrystalline cellulose, such as Avicel™ PH101. The amount of filler in the mini-tablet may range from about 20 to about 50% filler, e.g., about 25%, about 30%, about 35%, about 40%, and about 45%, including all values and subranges therebetween.

Suitable glidants for mini-tablets include any glidants listed herein, such as colloidal silicon dioxide and talc. In some embodiments, the minitablet comprises from about 0.1 to about 5 wt % glidant, based on the total weight of the composition, e.g., about 0.5, about 1.0, about 1.5, about 2.0, about 2.5, about 2.5, about 3.0, about 3.5, about 4.0 or about 4.5 wt %, including all values and subranges therebetween.

Suitable lubricants for mini-tablets include any lubricants listed herein, such as stearic acid, and stearic acid salts, for example magnesium stearate. In some embodiments, the mini-tablets comprise from 0.1 to 5% lubricant, based on the total weight of the composition e.g., about 0.5, about 1.0, about 1.5, about 2.0, about 2.5, about 2.5, about 3.0, about 3.5, about 4.0 or about 4.5%, including all values and subranges therebetween.

The mini-tablets may be uncoated, or coated with one or more layers of coating. In some embodiments, the mini-tablets are at least partially enteric coated. In some embodiments, the enteric coating comprises a pH dependent polymer, for example a copolymer of the methacrylic acid and methacrylic acid ester such as a methacrylic acid copolymer, for example Eudragit e.g. Eudragit L30D55 which has a dissolution above pH 5.5. Other Eudragit polymers include: Eudragit L100-55 (dissolution above pH 5.5), Eudragit L100 (dissolution above pH 6.0) and Eudragit S100 (dissolution above pH 7.0). In some embodiments, the enteric coating comprises from 5 to 10% based on the total weight of the composition (dry polymer weight). The enteric coating can be produced by spraying the enteric polymer on top of the above-described core mini-tablet.

The enteric coating may further comprise a plasticizer, such as acetyl triethyl citrate or triethyl citrate, for example triethyl citrate (Citroflex). In some embodiments, the pharmaceutical compositions disclosed herein comprise from about 0.1 to about 5% plasticizer, based on the total weight of the composition e.g., about 0.5, about 1.0, about 1.5, about 2.0, about 2.5, about 2.5, about 3.0, about 3.5, about 4.0 or about 4.5%, including all values and subranges therebetween.

In some embodiments, the enteric coating further comprises a glidant to eliminate sticking during the film coating process such as talc, kaolin, or glycerol monostearate, for example glycerol monostearate (Imwitor 900K). In some embodiments, the glidant comprises from about 0.1 to about 5 wt %, based on the total weight of the composition e.g., about 0.5, about 1.0, about 1.5, about 2.0, about 2.5, about 2.5, about 3.0, about 3.5, about 4.0 or about 4.5%, including all values and subranges therebetween.

In some embodiments, the enteric coating further comprises a surfactant to provide homogeneous film mixtures, such as sodium lauryl sulfate, polyethylene glycol, or polysorbate, for example Polysorbate 80 (Crillet 4HP). In some embodiments, the surfactant comprises from about 0.1 to about 5 wt % based on the total weight of the composition including, about 0.5, about 1.0, about 1.5, about 2.0, about 2.5, about 2.5, about 3.0, about 3.5, about 4.0 or about 4.5%, including all values and subranges therebetween.

The mini-tablets may, if desired, further comprise one or more pharmaceutically acceptable excipients. Suitable pharmaceutically acceptable excipients may include colors, flavors (such as menthol), sweeteners (such as mannitol), preservatives, stabilizers, antioxidants and any other excipients known to those skilled in the art.

### Timed Pulsatile Release

In some embodiments, the delayed release component is formulated to release zolmitriptan in a pulse at least 30 minutes after oral administration. The timed, pulsatile release systems described herein are capable of providing one or more immediate release pulses of zolmitriptan after predetermined delay times or at specific sites, to improve absorption of the drug.

In some embodiments, the timed pulsatile release system comprises a membrane surrounding the active core, wherein the membrane comprising swelling agents (e.g., low-substituted hydroxypropylcellulose, crospovidone, crosslinked carboxymethylcellulose, sodium starch glycolate), e.g., as disclosed in U.S. Pat. No. 4,871,549, which is herein incorporated by reference in its entirety.

In other embodiments, the timed pulsatile release system comprises a core material (such as a polysaccharide or a crosslinked protein and a disintegrant that swell on exposure to body fluids or water) and a rigid membrane surrounding the core comprising hydrophobic and hydrophilic polymers that bursts rapidly releasing the active when the core swells, e.g., as disclosed in U.S. Pat. No. 6,531,152, which is herein incorporated by reference in its entirety.

In some embodiments, the timed pulsatile release component comprises an inner barrier coating and an outer lag-time (i.e., delayed release) coating. In some embodiments, the inner barrier coating comprises a water insoluble polymer in combination with a water-soluble/pore-forming polymer. In some embodiments, the outer lag-time coating comprises a water-insoluble polymer in combination with an enteric polymer.

Non-limiting examples of suitable water insoluble polymers for the inner barrier coating include ethylcellulose, polyvinyl acetate (for example, Kollicoat SR #30D from BASF), cellulose acetate, cellulose acetate butyrate, neutral copolymers based on ethyl acrylate and methylmethacrylate, copolymers of acrylic and methacrylic acid esters with quaternary ammonium groups such as Eudragit NE, RS and RS30D, RL or RL30D and the like.

Non-limiting examples of suitable water-soluble/pore-forming polymer for the inner barrier coating include polyvinylpyrrolidone, methylcellulose, hydroxypropylcellulose, hydroxypropyl methylcellulose, polyethylene glycol, and mixtures thereof.

In some embodiments, the ratio of water insoluble polymer and water-soluble/pore-forming polymer for the inner barrier is from about 99:1 to about 1:99, including, about 90:10, about 80:20, about 70:30, about 60:40, about 50:50, about 40:60, about 30:70, about 20:80, about 10:90, including all values and subranges therebetween.

In some embodiments, the inner barrier coating comprises from about 1 wt % to about 20 wt %, including, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, and about 19 wt %, including all values and subranges therebetween.

Non-limiting examples of suitable enteric polymers for the outer lag-time coating include esters of cellulose and its derivatives (cellulose acetate phthalate, hydroxypropyl methylcellulose phthalate, hydroxypropyl methylcellulose acetate succinate), polyvinyl acetate phthalate, pH-sensitive methacrylic acid-methylmethacrylate copolymers and shellac. These polymers may be used as a dry powder or an aqueous dispersion. Some commercially available materials that may be used are methacrylic acid copolymers sold under the trademark Eudragit (L100, S100, L30D) manufactured by Rohm Pharma, Cellacefate (cellulose acetate phthalate) from Eastman Chemical Co., Aquateric (cellulose acetate phthalate aqueous dispersion) from FMC Corp. and Aqoat (hydroxypropyl methylcellulose acetate succinate aqueous dispersion) from Shin Etsu K.K.

In some embodiments, the outer lag-time membrane comprises a plasticized mixture of a water-insoluble polymer and an enteric polymer wherein the water-insoluble polymer and the enteric polymer may be present at a weight ratio in the range of about 10:1 to about 1:2, including, about 9:1, about 8:1, about 7:1, about 6.1, about 5:1, about 4:1, about 3:1, about 2:1, and about 1:1, including all values and subranges therebetween, and typically about 3:1 to about 1:1. In other embodiments, the water-insoluble polymer comprises from about 10% to about 99% of the plasticized mixture of a water-insoluble polymer and an enteric polymer, including, about 15%, about 20%, about 25%, about 30%, about 35%, about 40%, about 45%, about 50%, about 55%, about 60%, about 65%, about 70%, about 75%, about 80%, about 85%, about 90%/o, about 91%, about 92%, about 93%, about 94%, about 95%, about 96%, about 97%, about 98%, including all values and subranges therebetween. The total weight of the lag coating may vary from about 30% to about 60% by weight based on the total weight of the coated bead, including, about 35%, about 40%, about 45%, about 50%, and about 55%, including all values and subranges therebetween.

Both enteric and water-insoluble polymers used in forming the outer lag-time coating may be plasticized. Representative examples of plasticizers that may be used to plasticize the outer lag-time coating include triacetin, tributyl citrate, triethyl citrate, acetyl tri-n-butyl citrate, diethyl phthalate, castor oil, dibutyl sebacate, acetylated monoglycerides, acetylated diglycerides and the like or mixtures thereof. The plasticizer, when present, comprises about 3% to 30 wt. % of the polymer wt, e.g., about 5%, about 10%, about 15%, about 20%, about 25%, including all values and subranges therebetween. The type of plasticizer and its content depends on the polymer or polymers and nature of the coating system (e.g., aqueous or solvent based, solution or dispersion based and the total solids).

The amount and ratio of the components in the inner barrier coating and the outer lag-time coating can be modified to provide for a desired release time, e.g., within about 1, within about 2, or within about 3 hours. Further, the time-pulsatile release system may be formulated as particles having a diameter of at least 2 mm (e.g., as mini-tablet described above having a diameter of from about 2 mm to about 5 mm) to provide for sufficient residence time in the stomach.

Examples of timed, pulsatile release systems can be found in, e.g., U.S. Pat. Nos. 4,871,549, 6,531,152, 6,287,599, 6,627,223, 6,663,888, 9,161,918, and 9,161,919, each of which are herein incorporated by reference in its entirety.

### Soft-Gel Capsule

In some embodiments, the delayed release component is formulated as a soft-gel capsule. Soft-gel capsules are known in the art and may be prepared from gelatin and derivatives thereof. In some embodiments, the soft-gel capsule is formulated to dissolve at the acidic pH of the stomach.

In some embodiments, the soft-gel capsule is filled with a pharmaceutically acceptable liquid comprising drug particles dissolved or suspended therein.

Non-limiting examples of suitable pharmaceutically acceptable liquids include oils or polyols, such as glycerin and its homologous polyhydric alcohols, and their esters, and polycarbonates or syrups; waxes which are liquid at room temperature, e.g. Labrafac Lipophile, Labrafil M1944CS, Labrasol, Transcutol, Peceol, and Plurol manufactured by Gatefosse, Elmsford, N.Y., USA: triethyl citrate, acetyl triethyl citrate, tri-n-butyl citrate, or acetyltri-n-butyl citrate manufactured by Morflex, Greensboro, N.C., USA; glyceryl triacetate; or other liquids which do not solubilize gelatin. Mixtures of these can be used as well. In some embodiments, the pharmaceutically acceptable liquid is a vegetable oil or mineral oil. In some embodiments, the vegetable oil is selected from the group consisting of castor bean oil, coconut oil, peanut oil, palm kernel oil, canola oil, avocado oil, evening primrose oil, rice bran oil, borage oil, sunflower oil, soybean oil, palm oil, corn oil, and safflower oil and mixtures thereof.

### Depot Formulations

In some embodiments, the delayed release component comprises an injectable lipid-based liquid with dissolved zolmitriptan that spontaneously forms a controlled release liquid crystal gel matrix in aqueous environments (e.g., after it is injected subcutaneously into a patient in need thereof). Without being bound by any theory, such lipid solutions transform into a liquid crystalline gel in situ upon contact with fluids at the site of injection, which effectively encapsulates the zolmitriptan. In the depot formulations of the present disclosure, the zolmitriptan is slowly released as the liquid crystalline matrix degrades in the aqueous environment (e.g., tissue or bodily fluid). Injectable lipid-based liquid depot formulations are described in U.S. Pat. Nos. 8,236,292, 8,097,239, and 9,585,959, the contents of which are hereby incorporated by reference in their entireties.

The release rate of the zolmitriptan from the depot can be controlled over a period of a number of hours, days, or weeks depending on the composition by using methods known to those skilled to those skilled in the art, and described, e.g., in U.S. Pat. Nos. 8,236,292, and 9,585,959. In some embodiments, the suitable lipid-based liquids comprise appropriate neutral lipids, and/or amphipathic lipids and/or polar lipids or combinations thereof. In some embodiments, the suitable lipid-based liquids comprise neutral diacyl lipids and/or phospholipids such as those described in U.S. Pat. Nos. 8,097,239, and 9,585,959. In some embodiments, such preparations are administered subcutaneously (e.g., injected by syringe). In some embodiments of the present disclosure, the depot formulations use the FluidCrystal® injection depot described e.g., in U.S. Pat. Nos. 8,236,292, 8,097,239, and 9,585,959, the contents of which are hereby incorporated by reference in their entireties. Thus, in some embodiments, the depot formulation is a FluidCrystal® extended release formulation of zolmitriptan.

In some embodiments, the delayed release component comprises an injectable aqueous suspension of multivesicular liposomes (MVL). Injectable aqueous suspensions of MVLs are described in U.S. Pat. Nos. 5,723,147, 5,766,627, 5,891,467, 5,997,899, 6,793,938, and 9,585,838, the contents of which are hereby incorporated by reference in their entireties. Pharmaceutical compositions comprising MVLs for parenteral administration may be obtained, for example, by a double emulsion process forming a water-in-oil-in-water emulsion. The liposome particles are generally in the 10-30 μm diameter size range. In some embodiments, multivesicular liposomes comprise neutral lipids and amphipathic lipids. In some embodiments, multivesicular liposomes comprise phospholipids, cholesterol, and triglycerides. In some embodiments, phospholipids, such as dioleylphosphatidylcholine, comprise the major constituent of the lipid bilayer membrane. In some embodiments, charged phospholipids (dipalmitoylphosphatidylglycerol) are included in the lipid bilayer membrane to help prevent aggregation of the liposomes by charge repulsion. In some embodiments, cholesterol is used e.g., to provide mechanical stabilization of the lipid bilayer membrane. For parenteral administration, the drug substance can be formulated with the MVLs to provide a carrier matrix of microscopic, spherical, lipid-based particles composed of a honeycomb of numerous, non-concentric, internal aqueous chambers containing the encapsulated zolmitriptan. In some embodiments, each chamber is separated from adjacent chambers by lipid membranes. Following injection, such preparations release the zolmitriptan over an extended period of time due to e.g., erosion and/or reorganization of the lipid membranes. The release rate of the zolmitriptan from the MVLs can be controlled using methods known to those skilled to those skilled in the art, and described, for example, in U.S. Pat. Nos. 5,723,147, 5,766,627, 5,891,467, 6,793,938 and 9,585,838. In some embodiments, such compositions provide, for example, an immediate-release dose followed by sustained delivery depending on the composition. In some embodiments, such preparations are administered intravenously, subcutaneously, intramuscularly, or intrathecally (e.g. injected by pen systems or by syringe (using for example, a 25 G needle or larger to maintain the structural integrity of the liposomes)). Illustratively, systemic delivery by releasing zolmitriptan into the bloodstream via the interstitial space is an effective mode of administration. In some embodiments of the present disclosure, the depot formulations are prepared using the DepoFoam® technology platform described e.g., in U.S. Pat. Nos. 5,723,147, 5,766,627, 5,891,467, 5,997,899, 6,793,938, and 9,585,838. Thus, in some embodiments, the depot formulation is a zolmitriptan MVL injectable suspension.

In some embodiments, the delayed release component comprises an injectable hydrogel depot. Hydrogel depots are described, for example, in U.S. Pat. No. 8,084,045, U.S. Reissue Pat. No. RE46686, U.S. Pat. Nos. 7,659,365, and 8,206,744, the contents of which are hereby incorporated by reference in their entireties. For parenteral administration, appropriate hydrogel compositions include those comprising an amphiphilic polymer that allows for non-covalent capture of zolmitriptan and spontaneous formation of a colloidal suspension of stable nanoparticles (e.g. 10-50 nm) in water (e.g., under isotonic conditions). In some embodiments, the polymer is any polymer described herein, or in U.S. Pat. No. 8,084,045, U.S. Reissue Pat. No. RE46686, U.S. Pat. Nos. 7,659,365, and 8,206,744. In some embodiments, the polymer is a water-soluble biodegradable, amphiphilic copolymer. Without being bound by any theory, the sustained drug release is based on reversible drug interactions with hydrophobic nanodomains. In some embodiments, parenteral hydrogel depot injections provide a modified release rate of zolmitriptan. Hydrogel depot preparations may be obtained, for example, from self-assembling, poly-amino acid nanoparticles, made of a poly-Glu backbone grafted with hydrophobic a-tocopherol molecules using methods known to those skilled in the art, and described, for example, in U.S. Pat. No. 8,084,045, and U.S. Reissue Pat. No. RE46686EL. In some embodiments, hydrogel depot injections are administered subcutaneously. In some embodiments of the present disclosure, the depot formulations are prepared using the Medusa® Platform technology platform described e.g., in the figure below, and in U.S. Pat. No. 8,084,045, U.S. Reissue Pat. No. RE46686E1, U.S. Pat. Nos. 7,659,365, and 8,206,744, the contents of which are hereby incorporated by reference in their entireties. Thus, in some embodiments, the depot formulation is an injectable hydrogel extended release formulation of zolmitriptan.

In some embodiments, the delayed release component comprises a polymeric depot system. Injectable polymeric depots are described, for example, in U.S. Pat. Nos. 10,300,019, 8,674,033, and 8,481,651, the contents of which are hereby incorporated by reference in their entireties. Such preparations can be obtained, by using suitable monomers, to form a biodegradable polymer matrix in which zolmitriptan is entrapped. Exemplary monomers are described in U.S. Pat. Nos. 10,300,019, 8,674,033, and 8,481,651. When rehydrated, the polymers swell and the zolmitriptan is released by diffusion. Release time can be regulated for a period from hours, to days, and up to months by methods known to those skilled in the art and described, for example, in U.S. Pat. Nos. 10,300,019, 8,674,033, and 8,481,651. In some embodiments, the polymer matrix comprises poly (lactic acid) (PLA) and/or poly (lactic-co-glycolic acid) (PLGA), or multi-block copolymers composed of building blocks lactide, glycolide, ε-caprolactone and polyethylene glycol. In some embodiments, a near neutral pH is maintained in the polymer matrix. In some embodiments the block copolymer is represented by:

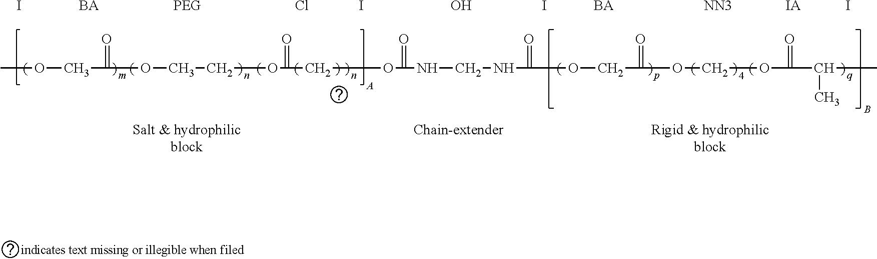

In some embodiments of the present disclosure, the depot formulations are prepared using the SynBiosys® Platform technology platform described in, e.g., U.S. Pat. Nos. 10,300,019, 8,674,033, and 8,481,651. Thus, in some embodiments, the depot formulation is a polymeric extended release depot formulation of zolmitriptan.

In some embodiments, the delayed release component comprises a phospholipid depot composition. Injectable phospholipid depots are described in U.S. Pat. No. 9,517,202, the contents of which are hereby incorporated by reference in its entirety. In some embodiments, appropriate phospholipids may be formulated with zolmitriptan to provide a formulation that gels in situ upon parenteral injection. In some embodiments, such formulations can also be used as a suspending agent for microspheres. In some embodiments, the gels can be substantially uniform or one-phase. Exemplary phospholipid compositions are described in U.S. Pat. No. 9,517,202 and can comprise 20-80% by weight, 25 to 70% by weight, or 30 to 60% by weight of a phospholipid such as 25%, 30%, 35%, 40%, 45%, 50%, 55%, or 60% by weight of a phospholipid or a mixture of phospholipids including all values and subranges therebetween. In some embodiments, the phospholipid is a non-liposomal phospholipid. In some embodiments, the phospholipid depot formulation is tuned to provide a customizable release profile over days, or weeks depending on the composition by methods known to those skilled in the art, and described in U.S. Pat. No. 9,517,202. In some embodiments, the depot formulations are administered parenterally (e.g., by subcutaneous or intramuscular injection or by injection or instillation into body tissues, vessels or cavities). In some embodiments of the present disclosure, the depot formulations are prepared using the PG Depot™ injectable depots described in e.g., U.S. Pat. No. 9,517,202. Thus, in some embodiments, the depot formulation is a phospholipid depot extended release formulation of zolmitriptan.

### Administration and Dosing

The present disclosure provides methods for treating the symptoms associated with autism by administering an effective amount of a zolmitriptan composition of the present disclosure to a patient in need thereof.

The present disclosure provides methods for treating aggression in patients with dementia by administering an effective amount of a zolmitriptan composition of the present disclosure to a patient in need thereof.

The present disclosure provides methods for treating aggression in Alzheimer's patients by administering an effective amount of a zolmitriptan composition of the present disclosure to a patient in need thereof.

In one aspect, the present disclosure provides treatment methods by administering (such as, by oral administration) an effective amount of a zolmitriptan composition of the present disclosure to a patient in need thereof. An effective amount depends on the treated condition and is an amount sufficient to eliminate or significantly reduce at least one symptom of the condition or to alleviate those symptoms (for example, the symptoms of autism spectrum disorder, or aggression in a patient with dementia, such as an Alzheimer's patients).

The following enumerated embodiments disclose methods for treating the symptoms of AD. These enumerated methods are also suitable for treating other conditions characterized by aggression (e.g., aggression in patients with dementia, such as an Alzheimer's patients). The enumerated embodiments (e.g., dose, PK parameters and administrations) described herein for treatment of symptoms of autism spectrum disorder equally apply for the treatment of conditions characterized by aggression (e.g., aggression in Alzheimer's patients). Thus, the present disclosure contemplates embodiments where the following doses, PK parameters and administrations are used to treat aggression in patients with dementia, such as Alzheimer's patients.

According to some embodiments of the present disclosure, administering the zolmitriptan compositions of the present disclosure provides a statistically significant therapeutic effect. In one embodiment, the statistically significant therapeutic effect is determined based on one or more standards or criteria provided by one or more regulatory agencies in the United States, e.g., FDA or other countries. In another embodiment, the statistically significant therapeutic effect is determined based on results obtained from regulatory agency approved clinical trial set up and/or procedure.

In some embodiments, the statistically significant therapeutic effect is determined based on a patient population of at least 20, 50, 60, 100, 200, 300, 400, 500, 600, 700, 800, 900, 1000 or 2000. In some embodiments, the statistically significant therapeutic effect is determined based on data obtained from randomized and double-blinded clinical trial set up. In some embodiments, the statistically significant therapeutic effect is determined based on data with a p value of less than or equal to about 0.05, 0.04, 0.03, 0.02 or 0.01. In some embodiments, the statistically significant therapeutic effect is determined based on data with a confidence interval greater than or equal to 95%, 96%, 97%, 98% or 99%.

In some embodiments, the statistically significant therapeutic effect is determined by a randomized double-blind clinical trial of patients treated with zolmitriptan or a pharmaceutically acceptable salt thereof and optionally in combination with standard care. The methods for determining a therapeutic effect will depend on the treated condition.

In general, statistical analysis can include any suitable method permitted by a regulatory agency, e.g., FDA in the US or Europe or any other country. In some embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM) and Cox regression analysis.

According to the present disclosure, a composition of the present disclosure is administered on a once a day, twice a day, or three-times-a-day basis to provide effective relief of the symptoms of a treated condition (for example, the symptoms of autism spectrum disorder or aggression in patients with dementia, such as an Alzheimer's patients).

In some embodiments, the doses provided herein refer to the amount of zolmitriptan administered per day to treat the symptoms of autism spectrum disorder. In some embodiments, the doses provided herein refer to the amount of zolmitriptan administered per day to treat aggression in patients with dementia. In some embodiments, the doses provided herein refer to the amount of zolmitriptan administered per day to treat aggression in Alzheimer's patients. In some embodiments, a total daily dose of zolmitriptan is about 7.5 mg, about 10 mg, about 12 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, and about 150 mg. In some embodiments, the daily dose of zolmitriptan is about 12 mg, about 24 mg, about 36 mg, or about 48 mg. In some embodiments, the daily dose of zolmitriptan is about 12 mg, about 24 mg, about 36 mg, about 48 mg, or about 72 mg.

In some embodiments, the total daily dose of zolmitriptan is from about 7.5 mg to about 150 mg, including about 7.5 mg, 10 mg, about 12 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, and about 150 mg, including all ranges there between. In some embodiments, the total daily dose of zolmitriptan is from about 60 mg to about 120 mg, including about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg, including all ranges there between. In some embodiments, the total daily dose of zolmitriptan is from about 7.5 mg to about 90 mg. In some embodiments, the total daily dose of zolmitriptan is from about 7.5 mg to about 50 mg. In some embodiments, the total daily dose of zolmitriptan is from about 10 mg to about 30 mg. In embodiments, the total daily dose of zolmitriptan is from about 12 mg to about 72 mg. In embodiments, the total daily dose of zolmitriptan is from about 24 mg to about 72 mg. In embodiments, the total daily dose of zolmitriptan is from about 48 mg to about 72 mg.

In some embodiments, the total daily dose of zolmitriptan is at least about 2.5 mg a day for the treatment of the symptoms of autism spectrum disorder. In some embodiments, the total daily dose is at least about 5 mg a day for the treatment of the symptoms of autism spectrum disorder. In some embodiments, the total daily dose of zolmitriptan is at least about 7.5 mg a day for the treatment of the symptoms of autism spectrum disorder. In some embodiments, the total daily dose of zolmitriptan is at least about 10 mg a day for the treatment of the symptoms of autism spectrum disorder. In some embodiments, the total daily dose of zolmitriptan is at least about 12 mg a day for the treatment of the symptoms of autism spectrum disorder. In some embodiments, the total daily dose of zolmitriptan is at least about 15 mg a day for the treatment of the symptoms of autism spectrum disorder. In some embodiments, the total daily dose of zolmitriptan is at least about 20 mg a day for the treatment of the symptoms of autism spectrum disorder. In some embodiments, the total daily dose of zolmitriptan is at least about 24 mg a day for the treatment of the symptoms of autism spectrum disorder. In some embodiments, the total daily dose of zolmitriptan is at least about 25 mg a day for the treatment of the symptoms of autism spectrum disorder. In some embodiments, the total daily dose of zolmitriptan is at least about 30 mg a day for the treatment of the symptoms of autism spectrum disorder. In some embodiments, the total daily dose of zolmitriptan is at least about 35 mg a day for the treatment of the symptoms of autism spectrum disorder. In some embodiments, the total daily dose of zolmitriptan is at least about 40 mg a day for the treatment of the symptoms of autism spectrum disorder. In some embodiments, the total daily dose of zolmitriptan is at least about 45 mg a day for the treatment of the symptoms of autism spectrum disorder. In some embodiments, the total daily dose of zolmitriptan is at least about 48 mg a day for the treatment of the symptoms of autism spectrum disorder. In some embodiments, the total daily dose of zolmitriptan is at least about 50 mg a day for the treatment of the symptoms of autism spectrum disorder. In some embodiments, the total daily dose of zolmitriptan is at least about 55 mg a day for the treatment of the symptoms of autism spectrum disorder. In some embodiments, the total daily dose of zolmitriptan is at least about 60 mg a day for the treatment of the symptoms of autism spectrum disorder. In some embodiments, the total daily dose of zolmitriptan is at least about 65 mg a day for the treatment of the symptoms of autism spectrum disorder. In some embodiments, the total daily dose of zolmitriptan is at least about 70 mg a day for the treatment of the symptoms of autism spectrum disorder. In some embodiments, the total daily dose of zolmitriptan is at least about 72 mg a day for the treatment of the symptoms of autism spectrum disorder. In some embodiments, the total daily dose of zolmitriptan is at least about 75 mg a day for the treatment of the symptoms of autism spectrum disorder. In some embodiments, the total daily dose of zolmitriptan is at least about 80 mg a day for the treatment of the symptoms of autism spectrum disorder. In some embodiments, the total daily dose of zolmitriptan is at least about 85 mg a day for the treatment of the symptoms of autism spectrum disorder. In some embodiments, the total daily dose of zolmitriptan is at least about 90 mg a day for the treatment of the symptoms of autism spectrum disorder. In some embodiments, the total daily dose of zolmitriptan is at least about 95 mg a day for the treatment of the symptoms of autism spectrum disorder. In some embodiments, the total daily dose of zolmitriptan is at least about 100 mg a day for the treatment of the symptoms of autism spectrum disorder. In some embodiments, the total daily dose of zolmitriptan is at least about 105 mg a day for the treatment of the symptoms of autism spectrum disorder. In some embodiments, the total daily dose of zolmitriptan is at least about 110 mg a day for the treatment of the symptoms of autism spectrum disorder. In some embodiments, the total daily dose of zolmitriptan is at least about 115 mg a day for the treatment of the symptoms of autism spectrum disorder. In some embodiments, the total daily dose of zolmitriptan is at least about 120 mg a day for the treatment of the symptoms of autism spectrum disorder. In some embodiments, the total daily dose of zolmitriptan is at least about 125 mg a day for the treatment of the symptoms of autism spectrum disorder. In some embodiments, the total daily dose of zolmitriptan is at least about 130 mg a day for the treatment of the symptoms of autism spectrum disorder. In some embodiments, the total daily dose of zolmitriptan is at least about 135 mg a day for the treatment of the symptoms of autism spectrum disorder. In some embodiments, the total daily dose of zolmitriptan is at least about 140 mg a day for the treatment of the symptoms of autism spectrum disorder. In some embodiments, the total daily dose of zolmitriptan is at least about 145 mg a day for the treatment of the symptoms of autism spectrum disorder. In some embodiments, the total daily dose of zolmitriptan is at least about 150 mg a day for the treatment of the symptoms of autism spectrum disorder.

In some embodiments, the total daily dose of zolmitriptan is about 12 mg a day for the treatment of the symptoms of ASD. In some embodiments, the total daily dose of zolmitriptan is about 24 mg a day for the treatment of the symptoms of ASD. In some embodiments, the total daily dose of zolmitriptan is about 36 mg a day for the treatment of the symptoms of ASD. In some embodiments, the total daily dose of zolmitriptan is about 48 mg a day for the treatment of the symptoms of ASD. In some embodiments, the total daily dose of zolmitriptan is about 60 mg a day for the treatment of the symptoms of ASD. In some embodiments, the total daily dose of zolmitriptan is about 65 mg a day for the treatment of the symptoms of ASD. In some embodiments, the total daily dose of zolmitriptan is about 70 mg a day for the treatment of the symptoms of ASD. In some embodiments, the total daily dose of zolmitriptan is about 72 mg a day for the treatment of the symptoms of ASD. In some embodiments, the total daily dose of zolmitriptan is about 75 mg a day for the treatment of the symptoms of ASD. In some embodiments, the total daily dose of zolmitriptan is about 80 mg a day for the treatment of the symptoms of ASD. In some embodiments, the total daily dose of zolmitriptan is about 85 mg a day for the treatment of the symptoms of ASD. In some embodiments, the total daily dose of zolmitriptan is about 90 mg a day for the treatment of the symptoms of ASD. In some embodiments, the total daily dose of zolmitriptan is about 95 mg a day for the treatment of the symptoms of ASD. In some embodiments, the total daily dose of zolmitriptan is about 100 mg a day for the treatment of the symptoms of ASD. In some embodiments, the total daily dose of zolmitriptan is about 105 mg a day for the treatment of the symptoms of ASD. In some embodiments, the total daily dose of zolmitriptan is about 110 mg a day for the treatment of the symptoms of ASD. In some embodiments, the total daily dose of zolmitriptan is about 115 mg a day for the treatment of the symptoms of ASD. In some embodiments, the total daily dose of zolmitriptan is about 120 mg a day for the treatment of the symptoms of ASD.

In some embodiments, about 12 mg of zolmitriptan once a day (or twice a day or three times a day) is selected to treat the symptoms of ASD. In some embodiments, about 24 mg of zolmitriptan once a day (or twice a day or three times a day) is selected to treat the symptoms of ASD. In some embodiments, about 36 mg of zolmitriptan once a day (or twice a day or three times a day) is selected to treat the symptoms of ASD. In some embodiments, about 48 mg of zolmitriptan once a day (or twice a day or three times a day) is selected to treat the symptoms of ASD. In some embodiments, about 60 mg of zolmitriptan once a day (or twice a day or three times a day) is selected to treat the symptoms of ASD. In some embodiments, about 65 mg of zolmitriptan once a day (or twice a day or three times a day) is selected to treat the symptoms of ASD. In some embodiments, about 70 mg of zolmitriptan once a day (or twice a day or three times a day) is selected to treat the symptoms of ASD. In some embodiments, about 72 mg of zolmitriptan once a day (or twice a day or three times a day) is selected to treat the symptoms of ASD. In some embodiments, about 75 mg of zolmitriptan once a day (or twice a day or three times a day) is selected to treat the symptoms of ASD. In some embodiments, about 80 mg of zolmitriptan once a day (or twice a day or three times a day) is selected to treat the symptoms of ASD. In some embodiments, about 85 mg of zolmitriptan once a day (or twice a day or three times a day) is selected to treat the symptoms of ASD. In some embodiments, about 90 mg of zolmitriptan once a day (or twice a day or three times a day) is selected to treat the symptoms of ASD. In some embodiments, about 95 mg of zolmitriptan once a day (or twice a day or three times a day) is selected to treat the symptoms of ASD. In some embodiments, about 100 mg of zolmitriptan once a day (or twice a day or three times a day) is selected to treat the symptoms of ASD. In some embodiments, about 105 mg of zolmitriptan once a day (or twice a day or three times a day) is selected to treat the symptoms of ASD. In some embodiments, about 110 mg of zolmitriptan once a day (or twice a day or three times a day) is selected to treat the symptoms of ASD. In some embodiments, about 115 mg of zolmitriptan once a day (or twice a day or three times a day) is selected to treat the symptoms of ASD. In some embodiments, about 120 mg of zolmitriptan once a day (or twice a day or three times a day) is selected to treat the symptoms of ASD.

In some embodiments, zolmitriptan or a pharmaceutically acceptable salt thereof is administered on a once a day or twice a day or three times a day basis for at least one day, for example, about 1 day, about 2 days, about 3 days, about 4 days, about 5 days, about 6 days, about 7 days, about 8 days, about 9 days, about 10 days, about 12 days, about 13 days and about 14 days.

In some embodiments, zolmitriptan or a pharmaceutically acceptable salt thereof is administered on a once a day or twice a day basis for at least a week, for example, about a week, about 2 weeks, about 3 weeks, about 4 weeks, about 5 weeks, about 6 weeks, about 7 weeks, about 8 weeks, about 9 weeks, about 10 weeks, about 12 weeks, about 18 weeks, about 24 weeks, and about 50 weeks.

In some embodiments, zolmitriptan or a pharmaceutically acceptable salt thereof is administered on a once a day or twice a day basis for at least about a week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 12 weeks, at least about 16 weeks, at least about 18 weeks, at least about 24 weeks, and at least about 50 weeks.

In some embodiments, at least about 7.5 mg or about 7.5 mg of zolmitriptan or a pharmaceutically acceptable salt thereof is administered on a once a day or twice a day basis for at least a week. In some embodiments, at least about 12 mg or about 12 mg of zolmitriptan or a pharmaceutically acceptable salt thereof is administered on a once a day or twice a day basis for at least a week. In some embodiments, at least about 24 mg or about 24 mg of zolmitriptan or a pharmaceutically acceptable salt thereof is administered on a once a day or twice a day basis for at least a week. In some embodiments, at least about 36 mg or about 36 mg of zolmitriptan or a pharmaceutically acceptable salt thereof is administered on a once a day or twice a day basis for at least a week. In some embodiments, at least about 48 mg or about 48 mg of zolmitriptan or a pharmaceutically acceptable salt thereof is administered on a once a day or twice a day basis for at least a week. In some embodiments, at least about 60 mg or about 60 mg of zolmitriptan or a pharmaceutically acceptable salt thereof is administered on a once a day or twice a day basis for at least a week. In some embodiments, at least about 65 mg or about 65 mg of zolmitriptan or a pharmaceutically acceptable salt thereof is administered on a once a day or twice a day basis for at least a week. In some embodiments, at least about 70 mg or about 70 mg of zolmitriptan or a pharmaceutically acceptable salt thereof is administered on a once a day or twice a day basis for at least a week. In some embodiments, at least about 72 mg or about 72 mg of zolmitriptan or a pharmaceutically acceptable salt thereof is administered on a once a day or twice a day basis for at least a week. In some embodiments, at least about 75 mg or about 75 mg of zolmitriptan or a pharmaceutically acceptable salt thereof is administered on a once a day or twice a day basis for at least a week. In some embodiments, at least about 80 mg or about 80 mg of zolmitriptan or a pharmaceutically acceptable salt thereof is administered on a once a day basis for at least a week. In some embodiments, at least about 85 mg or about 85 mg of zolmitriptan or a pharmaceutically acceptable salt thereof is administered on a once a day basis for at least a week. In some embodiments, at least about 90 mg or about 90 mg of zolmitriptan or a pharmaceutically acceptable salt thereof is administered on a once a day basis for at least a week. In some embodiments, at least about 95 mg or about 95 mg of zolmitriptan or a pharmaceutically acceptable salt thereof is administered on a once a day basis for at least a week. In some embodiments, at least about 100 mg or about 100 mg of zolmitriptan or a pharmaceutically acceptable salt thereof is administered on a once a day basis for at least a week. In some embodiments, at least about 105 mg or about 105 mg of zolmitriptan or a pharmaceutically acceptable salt thereof is administered on a once a day basis for at least a week. In some embodiments, at least about 110 mg or about 110 mg of zolmitriptan or a pharmaceutically acceptable salt thereof is administered on a once a day basis for at least a week. In some embodiments, at least about 115 mg or about 115 mg of zolmitriptan or a pharmaceutically acceptable salt thereof is administered on a once a day basis for at least a week. In some embodiments, at least about 120 mg or about 120 mg of zolmitriptan or a pharmaceutically acceptable salt thereof is administered on a once a day basis for at least a week. In some embodiments, at least about 125 mg or about 125 mg of zolmitriptan or a pharmaceutically acceptable salt thereof is administered on a once a day or twice a day basis for at least a week. In some embodiments, at least about 130 mg or about 130 mg of zolmitriptan or a pharmaceutically acceptable salt thereof is administered on a once a day basis for at least a week. In some embodiments, at least about 135 mg or about 135 mg of zolmitriptan or a pharmaceutically acceptable salt thereof is administered on a once a day basis for at least a week. In some embodiments, at least about 140 mg or about 140 mg of zolmitriptan or a pharmaceutically acceptable salt thereof is administered on a once a day basis for at least a week. In some embodiments, at least about 145 mg or about 145 mg of zolmitriptan or a pharmaceutically acceptable salt thereof is administered on a once a day basis for at least a week. In some embodiments, at least about 150 mg or about 150 mg of zolmitriptan or a pharmaceutically acceptable salt thereof is administered on a once a day basis for at least a week.

In some embodiments, the methods described herein further comprise a step of titrating the dose of zolmitriptan (titration period), or a pharmaceutically acceptable salt thereof, for at least about one week to the maximum safe and effective dose. In some embodiments, the titration period is about 1 day, about 2 days, about 3 days, about 4 days, about 5 days, about 6 days, about 7 days, about 8 days, about 9 days, about 10 days, about 11 days, about 12 days, about 13 days, to about 14 days, including all values and subranges therebetween. In some embodiments, the titration period is at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days. In embodiments, the titration comprises increasing the daily dose of the zolmitriptan. In embodiments, the titration comprises increasing the daily dose of the zolmitriptan by about 5 mg to about 20 mg, including about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, including all values and subranges therebetween. In embodiments, the titration comprises increasing the daily dose of the zolmitriptan by about 12 mg daily.

In embodiments of the methods described herein, further comprise a step of down titrating the dose of zolmitriptan. In embodiments, the titration comprises decreasing the daily dose of the zolmitriptan by about 5 mg to about 20 mg, including about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, including all values and subranges therebetween. In embodiments, the titration comprises decreasing the daily dose of the zolmitriptan by about 12 mg daily.

In embodiments, the methods described herein comprise titrating the dose of zolmitriptan to a therapeutically effective dose and administering the therapeutically effective dose for about 1 day to about 20 weeks or more, including about 1 day, about 2 days, about 3 days, about 4 days, about 5 days, about 6 days, about 1 week, about 2 weeks, about 3 weeks, about 4 weeks, about 5 weeks, about 6 weeks, about 7 weeks, about 8 weeks, about 9 weeks, about 10 weeks, about 11 weeks, about 13 weeks, about 14 weeks, about 15 weeks, about 16 weeks, about 17 weeks, about 18 weeks, about 19 weeks, about 20 weeks or more, including all values and subranges therebetween.

In embodiments, the methods described herein comprise titrating the dose of zolmitriptan to a therapeutically effective dose and administering the therapeutically effective dose for at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 13 weeks, at least about 14 weeks, at least about 15 weeks, at least about 16 weeks, at least about 17 weeks, at least about 18 weeks, at least about 19 weeks, or at least about 20 weeks or more.

In embodiments, the methods described herein comprise titrating the dose of zolmitriptan to the maximum safe and effective dose and administering the maximum safe and effective dose for about 1 day to about 20 weeks or more, including about 1 day, about 2 days, about 3 days, about 4 days, about 5 days, about 6 days, about 1 week, about 2 weeks, about 3 weeks, about 4 weeks, about 5 weeks, about 6 weeks, about 7 weeks, about 8 weeks, about 9 weeks, about 10 weeks, about 11 weeks, about 13 weeks, about 14 weeks, about 15 weeks, about or 16 weeks, about 17 weeks, about 18 weeks, about 19 weeks, or about 20 weeks or more, including all values and subranges therebetween, 12251 In embodiments, the methods described herein comprise titrating the dose of zolmitriptan to the maximum safe and effective dose and administering the maximum safe and effective dose at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 13 weeks, at least about 14 weeks, least about 15 weeks, at least about 16 weeks, at least about 17 weeks, at least about 18 weeks, at least about 19 weeks, or at least about 20 weeks or more.

In embodiments of the methods of the present disclosure, the zolmitriptan composition is administered to a patient in need thereof (e.g., a patient with a Autism Spectrum Disorder (ASD) or an Alzheimer's patient with aggression) as an initial daily dose of about 12 mg zolmitriptan, and then the zolmitriptan dose is titrated to a therapeutically effective dose. In embodiments, the maximum daily dose is no more than about 72 mg zolmitriptan.

In embodiments of the methods of the present disclosure, the zolmitriptan composition is administered to a patient in need thereof (e.g., a patient with a Autism Spectrum Disorder (ASD) or an Alzheimer's patient with aggression) as an initial daily dose of at least about 12 mg zolmitriptan, and then the zolmitriptan dose is titrated to a therapeutically effective dose. In embodiments, the maximum daily dose is no more than about 72 mg zolmitriptan. 12281 In embodiments of the methods of the present disclosure, the zolmitriptan composition is administered to a patient in need thereof (e.g., a patient with a Autism Spectrum Disorder (ASD) or an Alzheimer's patient with aggression) as an initial daily dose of about 12 mg zolmitriptan, and then the dose of zolmitriptan is titrated to the maximum safe and effective dose. In embodiments, the maximum daily dose is no more than about 72 mg zolmitriptan.

In embodiments of the methods of the present disclosure, the zolmitriptan composition is administered to a patient in need thereof (e.g., a patient with a Autism Spectrum Disorder (ASD) or an Alzheimer's patient with aggression) as an initial daily dose of at least about 12 mg zolmitriptan, and then the dose of zolmitriptan is titrated to the maximum safe and effective dose. In embodiments, the maximum daily dose is no more than about 72 mg zolmitriptan.

In some embodiments, the present disclosure provides zolmitriptan compositions that are characterized on the basis of the pharmacokinetic (or PK) parameters that are observed following oral administration of the composition to a patient in need thereof. However, the Applicant contemplates that the same PK parameters which are achieved following oral administration of zolmitriptan may be achieved using other routes of drug administration, such as parenteral routes including but not limited to intravenous, intramuscular, intranasal, and transdermal. The PK parameters described herein may be achieved using any of the extended release compositions described herein.

In some embodiments, the pharmaceutical compositions provided herein provide plasma levels of zolmitriptan that correlate to one or more statistically significant therapeutic effects over an extended period of time, typically over a period of at least about 4 hours, at least about 6 hours, at least about 8 hours, at least about 10 hours, at least about 12 hours, at least about 14 hours, at least about 16 hours, at least about 18 hours, at least about 20 hours, at least about 22 hours, or at least about 24 hours to treat the symptoms of autism spectrum disorder in humans. In some embodiments, the pharmaceutical compositions provided herein provide plasma levels of zolmitriptan that correlate to one or more statistically significant therapeutic effects over a period of about 4-24 hours, including over a period of about 4 hours, about 6 hours, about 8 hours, about 10 hours, about 12 hours, about 14 hours, about 16 hours, about 18 hours, about 20 hours, about 22 hours to about 24 hours, including all values and subranges therebetween. In some embodiments, the plasma levels are steady state plasma levels. In some embodiments, when orally administered to a patient in need thereof the compositions of the present disclosure provide a therapeutically effective plasma concentration of zolmitriptan for about 8 h, about 10 h, about 12 h, or about 16 h following a single administration of the composition. In some embodiments, when orally administered to a patient in need thereof the compositions of the present disclosure provide a therapeutically effective plasma concentration of zolmitriptan for about 10 h, about 12 h, or about 16 h following a single administration of the composition. In some embodiments, when orally administered to a patient in need thereof the compositions of the present disclosure provide a therapeutically effective plasma concentration of zolmitriptan for at least about 8 h, at least about 10 h, at least about 12 h, or at least about 16 h following a single administration of the composition.

In some embodiments, the present compositions provide steady state plasma levels of zolmitriptan that correlate to one or more statistically significant therapeutic effects when administered on a once-a-day, twice-a-day, or three-times-a-day basis. In some embodiments, when orally administered to a patient in need thereof, the present compositions provide steady state plasma levels of zolmitriptan that provide statistically significant effects for at least about 12 hours following oral administration, including at least about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, and about 31 days. Methods of measuring statistically significant effects will depend on the treated condition and are known to those skilled in the art.

In some embodiments, when orally administered to a patient in need thereof on a once a day, twice a day, or three times a day basis the zolmitriptan compositions of the present disclosure provide a therapeutically effective steady state plasma levels of zolmitriptan ranging from about 3 ng/mL to about 85 ng/mL, including about 3 ng/ml, about 5 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 37 ng/mL, about 38 ng/mL, about 29 ng/mL, about 40 ng/mL, about 41 ng/mL, about 42 ng/mL, about 43 ng/mL, about 44 ng/mL, about 45 ng/mL, about 46 ng/mL, about 47 ng/mL, about 48 ng/mL, about 49 ng/mL, about 50 ng/mL, about 51 ng/mL, about 52 ng/mL, about 53 ng/mL, about 54 ng/mL, about 55 ng/mL, about 56 ng/mL, about 57 ng/mL, about 58 ng/mL, about 59 ng/mL, about 60 ng/mL, about 61 ng/mL, about 62 ng/mL, about 63 ng/mL, about 64 ng/mL, about 65 ng/mL, about 66 ng/mL, about 67 ng/mL, about 68 ng/mL, about 69 ng/mL, about 70 ng/mL, about 71 ng/mL, about 72 ng/mL, about 73 ng/mL, about 74 ng/mL, about 75 ng/mL, about 76 ng/mL, about 77 ng/mL, about 78 ng/mL, about 80 ng/mL, about 81 ng/mL, about 82 ng/mL, about 83 ng/mL, about 84 ng/mL, and about 85 ng/mL, including all ranges there between. In some embodiments, when orally administered to a patient in need thereof on a once a day, twice a day, or three times a day basis, the zolmitriptan compositions of the present disclosure provide therapeutically effective steady state plasma levels of zolmitriptan ranging from about 3 ng/ml to 85 ng/ml.

In some embodiments, when orally administered to a patient in need thereof the compositions of the present disclosure provide a blood plasma concentration of at least about 8-15 ng/mL for an extended period of time, including from at least about 8 ng/mL, at least about 9 ng/mL, at least about 10 ng/mL, at least about 11 ng/ml, at least about 12 ng/mL, at least about 13 ng/mL, at least about 14 ng/mL, to at least about 15 ng/mL, including all values and subranges therebetween for an extended period of time, typically at least about 6 hours, at least about 7 hours, at least about 8 hours, at least about 9 hours, at least about 10 hours, at least about 11 hours, or at least about 12 hours following a single administration of the composition. In some embodiments, when orally administered to a patient in need thereof the compositions of the present disclosure provide a blood plasma concentration of at least about 10 ng/mL for at least about 8 hours.

In embodiments of the present methods, plasma levels of about 10 ng/mL to about 25 ng/mL are provided by administering about 24 mg zolmitriptan compositions of the present disclosure, including about 10 ng/mL, about 11 ng/mL, about 12 ng/mL, about 13 ng/mL, about 14 ng/mL, about 15 ng/mL, about 16 ng/mL, about 17 ng/mL, about 17 ng/mL, about 18 ng/mL, about 19 ng/mL, about 20 ng/mL, about 21 ng/mL, about 22 ng/mL, about 23 ng/mL, about 24 ng/mL, to about 25 ng/mL, including all values and subranges therebetween. In embodiments of the present methods, plasma levels of about 25 ng/mL to about 60 ng/mL are provided by administering about 48 mg of zolmitriptan. In embodiments of the present methods, plasma levels of about 25 ng/mL to about 50 ng/mL are provided by administering about 48 mg of zolmitriptan. In embodiments of the present methods, plasma levels of about 25 ng/mL to about 45 ng/mL are provided by administering about 48 mg zolmitriptan compositions of the present disclosure, including about 25 ng/mL, about 26 ng/mL, about 27 g/mL, about 28 ng/mL, about 29 ng/mL, about 30 ng/mL, about 31 ng/mL, about 32 ng/mL, about 33 ng/mL, about 33 ng/mL, about 34 ng/mL, about 35 ng/mL, about 36 ng/mL, about 37 ng/mL, about 38 ng/mL, about 39 ng/mL, about 40 ng/mL, about 41 ng/mL, about 42 ng/mL, about 43 ng/mL, about 44 ng/mL, about 45 ng/mL, including all values and subranges therebetween. In embodiments of the present methods, plasma levels of about 40 ng/mL to about 65 ng/mL are provided by administering about 72 mg zolmitriptan compositions of the present disclosure, including about 40 ng/mL, about 41 ng/mL, about 42 ng/mL about 43 ng/mL, about 44 ng/mL, about 45 ng/mL, about 46 ng/mL, about 47 ng/mL, about 48 ng/mL, about 49 ng/mL, about 50 ng/mL, about 51 ng/mL, about 52 ng/mL, about 53 ng/mL, about 54 ng/mL, about 55 ng/mL, about 56 ng/mL, about 57 ng/mL, about 58 ng/mL, about 59 ng/mL, to about 60 ng/mL, including all values and subranges therebetween.

In some embodiments, when orally administered to a patient in need thereof on a once a day, twice a day, or three times a day basis the zolmitriptan compositions of the present disclosure provide a plasma level (e.g., after a single dose of the zolmitriptan composition) ranging from about 1 ng/mL to about 100 ng/mL, including about 1 ng/mL, about 2 ng/mL, about 3 ng/ml, about 5 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 37 ng/mL, about 38 ng/mL, about 29 ng/mL, about 40 ng/mL, about 41 ng/mL, about 42 ng/mL, about 43 ng/mL, about 44 ng/mL, about 45 ng/mL, about 46 ng/mL, about 47 ng/mL, about 48 ng/mL, about 49 ng/mL, about 50 ng/mL, about 51 ng/mL, about 52 ng/mL, about 53 ng/mL, about 54 ng/mL, about 55 ng/mL, about 56 ng/mL, about 57 ng/mL, about 58 ng/mL, about 59 ng/mL, about 60 ng/mL, about 61 ng/mL, about 62 ng/mL, about 63 ng/mL, about 64 ng/mL, about 65 ng/mL, about 66 ng/mL, about 67 ng/mL, about 68 ng/mL, about 69 ng/mL, about 70 ng/mL, about 71 ng/mL, about 72 ng/mL, about 73 ng/mL, about 74 ng/mL, about 75 ng/mL, about 76 ng/mL, about 77 ng/mL, about 78 ng/mL, about 80 ng/mL, about 81 ng/mL, about 82 ng/mL, about 83 ng/mL, about 84 ng/mL, about 85 ng/mL, about 86 ng/mL, about 87 ng/mL, about 88 ng/mL, about 89 ng/mL, about 90 ng/mL, about 91 ng/mL, about 92 ng/mL, about 93 ng/mL, about 94 ng/mL, about 95 ng/mL, about 96 ng/mL, about 97 ng/mL, about 98 ng/mL, about 99 ng/mL, and about 100 ng/mL, including all ranges there between. In embodiments, when orally administered to a patient in need thereof on a once a day, twice a day, or three times a day basis the zolmitriptan compositions of the present disclosure provide a plasma level ranging from about 40 ng/mL to about 60 ng/mL. In embodiments, when orally administered to a patient in need thereof on a once a day, twice a day, or three times a day basis the zolmitriptan compositions of the present disclosure provide a plasma level ranging from about 40 ng/mL to about 50 ng/mL.

In some embodiments, the therapeutically effective steady state plasma levels of zolmitriptan are provided by administering one or more compositions of the present disclosure to provide a daily dose of zolmitriptan or a pharmaceutically acceptable salt thereof of between about 7.5 mg and 150 mg. In some embodiments, the therapeutically effective steady state plasma levels of zolmitriptan is provided by administering a composition of the present disclosure containing between about 7.5 mg and about 90 mg of zolmitriptan or a pharmaceutically acceptable salt thereof once a day. In some embodiments, the therapeutically effective steady state plasma levels of zolmitriptan is provided by administering a composition of the present disclosure containing between about 12 mg and about 72 mg of zolmitriptan or a pharmaceutically acceptable salt thereof once a day. In some embodiments, the therapeutically effective steady state plasma levels of zolmitriptan is provided by administering a composition of the present disclosure containing between about 7.5 mg and about 50 mg of zolmitriptan or a pharmaceutically acceptable salt thereof once a day. In some embodiments, the therapeutically effective steady state plasma levels of zolmitriptan is provided by administering a composition of the present disclosure containing between about 2.5 mg and about 50 mg of zolmitriptan or a pharmaceutically acceptable salt thereof once a day. In some embodiments, the therapeutically effective steady state plasma levels of zolmitriptan is provided by administering a composition of the present disclosure containing between about 2.5 mg and about 50 mg of zolmitriptan or a pharmaceutically acceptable salt thereof twice a day. In some embodiments, the therapeutically effective steady state plasma levels of zolmitriptan is provided by administering a composition of the present disclosure containing between about 2.5 mg and about 50 mg of zolmitriptan or a pharmaceutically acceptable salt thereof three times a day. In some embodiments, the therapeutically effective steady state plasma levels of zolmitriptan is provided by administering a composition of the present disclosure containing between about 10 mg and about 30 mg of zolmitriptan or a pharmaceutically acceptable salt thereof once a day.

In some embodiments, administration of compositions of the present disclosure provide CSF levels of zolmitriptan that correlate to one or more statistically significant therapeutic effects. In some embodiments, the therapeutically effective CSF levels of zolmitriptan provided by the methods of the present disclosure range from about 0.05 ng/mL to about 2 ng/mL, including about 0.05 ng/mL, about 0.075 ng/mL, about 0.1 ng/mL, about 0.125 ng/mL, about 0.15 ng/mL, about 0.175 ng/mL, about 0.2 ng/mL, about 0.25 ng/mL, about 0.3 ng/mL, about 0.35 ng/mL, about 0.4 ng/mL, about 0.45 ng/mL, about 0.5 ng/mL, about 0.55 ng/mL, about 0.6 ng/mL, about 0.65 ng/mL, about 0.7 ng/mL, about 0.75 ng/mL, about 0.8 ng/mL, about 0.85 ng/mL, about 0.9 ng/mL, about 0.95 ng/mL, about 1 ng/mL, about 1.1 ng/mL, about 1.15 ng/mL, about 1.2 ng/mL, about 1.25 ng/mL, about 1.3 ng/mL, about 1.35 ng/mL, about 1.4 ng/mL, about 1.45 ng/mL, about 1.5 ng/mL, about 1.55 ng/mL, about 1.6 ng/mL, about 1.65 ng/mL, about 1.7 ng/mL, about 1.75 ng/mL, about 1.8 ng/mL, about 1.85 ng/mL, about 1.9 ng/mL, about 1.95 ng/mL, and about 2 ng/mL, including all values and ranges therebetween. In some embodiments, the therapeutically effective CSF levels of zolmitriptan provided by the methods of the present disclosure range from about 0.078 ng/mL to about 1.24 ng/mL. In some embodiments, the therapeutically effective CSF levels of zolmitriptan provided by the methods of the present disclosure range from about 0.103 ng/mL to about 0.93 ng/mL.

In some embodiments, the present compositions provide mean steady state AUC₀₋₂₄ₕ (expressed in terms of ng*hr/mL) levels of zolmitriptan that correlate to one or more statistically significant therapeutic effects when administered on a once-a-day, twice-a-day, or three times a day basis. In some embodiments, the present compositions provide mean steady state AUC₀₋₂₄ₕ levels of zolmitriptan that provide statistically significant effects for at least about 1 hour following oral administration to a patient in need thereof, including about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, and about 31 days or more, including all values and ranges in between. In embodiments, the present compositions provide mean steady state AUC₀₋₂₄ₕ levels of zolmitriptan that provide statistically significant effects for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about 31 days or more following oral administration to a patient in need thereof. In some embodiments, the present compositions provide mean steady state AUC₀₋₂₄ₕ levels of zolmitriptan that provide statistically significant effects for at least about 12 hours following oral administration to a patient in need thereof.

In some embodiments, the present compositions provide mean steady state AUC₀₋₂₄ₕ levels of zolmitriptan that provide statistically significant effects for a period of about or at least about 1-24 hours following administration to a patient in need thereof, including about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours and about 24 hours. In some embodiments, the present compositions provide mean steady state AUC₀₋₂₄ₕ levels of zolmitriptan that provide statistically significant effects for a period of about 12 hours to about 24 hours following administration to a patient in need thereof. In some embodiments, the present compositions provide mean steady state AUC₀₋₂₄ₕ levels of zolmitriptan that provide statistically significant effects for a period of at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, or at least about 24 hours. In some embodiments, the present compositions provide mean steady state AUC₀₋₂₄ₕ levels of zolmitriptan that provide statistically significant effects for a period of about 1 day to about 1 month following administration to a patient in need thereof, including at least about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 1 week, about 2 weeks, about 3 weeks, about 4 weeks, and about 5 weeks.

In some embodiments, the therapeutically effective mean steady state AUC₀₋₂₄ₕ levels of zolmitriptan provided by the zolmitriptan compositions of the present disclosure range from about 15 ng*hr/mL to about 1150 ng*hr/mL, including about 15 ng*hr/mL, 20 ng*hr/mL, 25 ng*hr/mL, 30 ng*hr/mL, 35 ng*hr/mL, 40 ng*hr/mL, 50 ng*hr/mL, 100 ng*hr/mL, 150 ng*hr/mL, 200 ng*hr/mL, 250 ng*hr/mL, 300 ng*hr/mL, about 400 ng*hr/mL, about 500 ng*hr/mL, about 600 ng*hr/mL, about 700 ng*hr/mL, about 800 ng*hr/mL, about 900 ng*hr/mL, about 1000 ng*hr/mL, about 1100 ng*hr/mL, and about 1150 ng*hr/mL, including all ranges and values there between. In some embodiments, the therapeutically effective mean steady state AUC₀₋₂₄ₕ levels of zolmitriptan provided by the zolmitriptan compositions of the present disclosure range from about 40 ng*hr/mL to about 110 ng*hr/mL, including about 40 ng*hr/mL, about 50 ng*hr/mL, about 60 ng*hr/mL, about 70 ng*hr/mL, about 80 ng*hr/mL, about 90 ng*hr/mL, about 100 ng*hr/mL, and about 110 ng*hr/mL, including all ranges and values there between. In some embodiments, the therapeutically effective mean steady state AUC₀₋₂₄ₕ levels of zolmitriptan provided by the zolmitriptan compositions of the present disclosure range from about 150 ng*hr/mL to about 450 ng*hr/mL, including about 150 ng*hr/mL, about 175 ng*hr/mL, about 200 ng*hr/mL, about 225 ng*hr/mL, about 250 ng*hr/mL, about 275 ng*hr/mL, about 300 ng*hr/mL, about 325 ng*hr/mL, about 350 ng*hr/mL, about 375 ng*hr/mL, about 400 ng*hr/mL, about 425 ng*hr/mL, and about 450 ng*hr/mL, including all ranges and values there between. In some embodiments, the therapeutically effective mean steady state AUC₀₋₂₄ₕ levels of zolmitriptan provided by the zolmitriptan compositions of the present disclosure range from about 600 ng*hr/mL to about 1150 ng*hr/mL, including about 600 ng*hr/mL, about 650 ng*hr/mL, about 700 ng*hr/mL, about 750 ng*hr/mL, about 800 ng*hr/mL, about 850 ng*hr/mL, about 900 ng*hr/mL, about 950 ng*hr/mL, about 1000 ng*hr/mL, about 1050 ng*hr/mL, about 1100 ng*hr/mL, and about 1150 ng*hr/mL, including all ranges and values there between.

In some embodiments, the present compositions provide an AUC₀₋₂₄ₕ (expressed in terms of ng*hr/mL) of about 20 ng·h/mL to about 1200 ng·h/mL following a single administration of the composition, including about 20 ng·h/mL, about 30 ng·h/mL, about 40 ng·h/mL, about 50 ng·h/mL, about 60 ng·h/mL, about 70 ng·h/mL, about 80 ng·h/mL, about 90 ng·h/mL, about 100 ng·h/mL, about 110 ng·h/mL, about 120 ng·h/mL, about 130 ng·h/mL, about 140 ng·h/mL, about 150 ng·h/mL, about 160 ng·h/mL, about 170 ng·h/mL, about 180 ng·h/mL, about 190 ng·h/mL, about 200 ng·h/mL, about 210 ng·h/mL, about 220 ng·h/mL, about 230 ng·h/mL, about 240 ng·h/mL, about 250 ng·h/mL, about 260 ng·h/mL, about 270 ng·h/mL, about 280 ng·h/mL, about 290 ng·h/mL, about 300 ng·h/mL, about 310 ng·h/mL, about 320 ng·h/mL, about 330 ng·h/mL, about 340 ng·h/mL, about 350 ng·h/mL, about 360 ng·h/mL, about 370 ng·h/mL, about 380 ng·h/mL, about 390 ng·h/mL, about 400 ng·h/mL, about 410 ng·h/mL, about 420 ng·h/mL, about 430 ng·h/mL, about 440 ng·h/mL, about 450 ng·h/mL, about 460 ng·h/mL, about 470 ng·h/mL, about 480 ng·h/mL, about 490 ng·h/mL, about 500 ng·h/mL, about 510 ng·h/mL, about 520 ng·h/mL, about 530 ng·h/mL, about 540 ng·h/mL, about 550 ng·h/mL, about 560 ng·h/mL, about 570 ng·h/mL, about 580 ng·h/mL, about 590 ng·h/mL, about 600 ng·h/mL, about 610 ng·h/mL, about 620 ng·h/mL, about 630 ng·h/mL, about 640 ng·h/mL, about 650 ng·h/mL, about 660 ng·h/mL, about 670 ng·h/mL, about 680 ng·h/mL, about 690 ng·h/mL, about 700 ng·h/mL, about 710 ng·h/mL, about 720 ng·h/mL, about 730 ng·h/mL, about 740 ng·h/mL, about 750 ng·h/mL, about 760 ng·h/mL, about 770 ng·h/mL, about 780 ng·h/mL, about 790 ng·h/mL, about 800 ng·h/mL, about 810 ng·h/mL, about 820 ng·h/mL, about 830 ng·h/mL, about 840 ng·h/mL, about 850 ng·h/mL, about 860 ng·h/mL, about 870 ng·h/mL, about 890 ng·h/mL, about 900 ng·h/mL, about 910 ng·h/mL, about 920 ng·h/mL, about 930 ng·h/mL, about 940 ng·h/mL, about 950 ng·h/mL, about 960 ng·h/mL, about 970 ng·h/mL, about 980 ng·h/mL, about 990 ng·h/mL, about 1000 ng·h/mL, about 1010 ng·h/mL, about 1020 ng·h/mL, about 1030 ng·h/mL, about 1040 ng·h/mL, about 1050 ng·h/mL, about 1060 ng·h/mL, about 1070 ng·h/mL, about 1080 ng·h/mL, about 1090 ng·h/mL, about 1100 ng·h/mL, about 1110 ng·h/mL, about 1120 ng·h/mL, about 1130 ng·h/mL, about 1140 ng·h/mL, about 1150 ng·h/mL, about 1160 ng·h/mL, about 1170 ng·h/mL, about 1180 ng·h/mL, about 1190 ng·h/mL, about 1200 ng·h/mL, including all values and subranges therebetween. In some embodiments, the present compositions provide an AUC₀₋₂₄ₕ of about 20 ng·h/mL to about 500 ng·h/mL following a single administration of the composition. In some embodiments, the present compositions provide an AUC₀₋₂₄ₕ of about 80 ng·h/mL to about 750 ng·h/mL following a single administration of the composition. In some embodiments, the present compositions provide an AUC₀₋₂₄ₕ of about 50 ng·h/mL to about 250 ng·h/mL following a single administration of the composition. In some embodiments, the present compositions provide an AUC₀₋₂₄ₕ of about 40 ng·h/mL to about 300 ng·h/mL, or about 150 ng·h/mL to about 250 ng·h/mL, or about 50 ng·h/mL to about 150 ng·h/mL following a single administration of the composition. In embodiments, the present compositions provide an AUC₀₋₂₄ₕ (expressed in terms of ng*hr/mL) of about 20 ng·h/mL to about 500 ng·h/mL following a single administration of the composition. In embodiments, the present compositions are dosed in the fed state. In embodiments, the compositions are dosed in the fasted state.

In some embodiments, the therapeutically effective mean steady state AUC₀₋₂₄ₕ levels of zolmitriptan is provided by administering one or more compositions of the present disclosure to provide a daily dose of zolmitriptan or a pharmaceutically acceptable salt thereof of between about 7.5 mg and about 150 mg. In some further embodiments, the therapeutically effective mean steady state AUC₀₋₂₄ₕ levels of zolmitriptan is provided by administering a composition of the present disclosure containing between about 7.5 mg and about 150 mg of zolmitriptan or a pharmaceutically acceptable salt thereof once a day. In other further embodiments, the therapeutically effective mean steady state AUC₀₋₂₄ₕ levels of zolmitriptan is provided by administering a composition of the present disclosure containing between about 3.75 mg and about 75 mg of zolmitriptan or a pharmaceutically acceptable salt thereof twice a day. In other further embodiments, the therapeutically effective mean steady state AUC₀₋₂₄ₕ levels of zolmitriptan is provided by administering a composition of the present disclosure containing between about 2.5 mg and about 50 mg of zolmitriptan or a pharmaceutically acceptable salt thereof three times a day.

In some embodiments, the present compositions provide a AUCₗₐₛₜ (expressed in terms of ng·hr/mL) of about 20 ng·h/mL to about 1200 ng·h/mL following a single administration of the composition, including about 20 ng·h/mL, about 30 ng·h/mL, about 40 ng·h/mL, about 50 ng·h/mL, about 60 ng·h/mL, about 70 ng·h/mL, about 80 ng·h/mL, about 90 ng·h/mL, about 100 ng·h/mL, about 110 ng·h/mL, about 120 ng·h/mL, about 130 ng·h/mL, about 140 ng·h/mL, about 150 ng·h/mL, about 160 ng·h/mL, about 170 ng·h/mL, about 180 ng·h/mL, about 190 ng·h/mL, about 200 ng·h/mL, about 210 ng·h/mL, about 220 ng·h/mL, about 230 ng·h/mL, about 240 ng·h/mL, about 250 ng·h/mL, about 260 ng·h/mL, about 270 ng·h/mL, about 280 ng·h/mL, about 290 ng·h/mL, about 300 ng·h/mL, about 310 ng·h/mL, about 320 ng·h/mL, about 330 ng·h/mL, about 340 ng·h/mL, about 350 ng·h/mL, about 360 ng·h/mL, about 370 ng·h/mL, about 380 ng·h/mL, about 390 ng·h/mL, about 400 ng·h/mL, about 410 ng·h/mL, about 420 ng·h/mL, about 430 ng·h/mL, about 440 ng·h/mL, about 450 ng·h/mL, about 460 ng·h/mL, about 470 ng·h/mL, about 480 ng·h/mL, about 490 ng·h/mL, about 500 ng·h/mL, about 510 ng·h/mL, about 520 ng·h/mL, about 530 ng·h/mL, about 540 ng·h/mL, about 550 ng·h/mL, about 560 ng·h/mL, about 570 ng·h/mL, about 580 ng·h/mL, about 590 ng·h/mL, about 600 ng·h/mL, about 610 ng·h/mL, about 620 ng·h/mL, about 630 ng·h/mL, about 640 ng·h/mL, about 650 ng·h/mL, about 660 ng·h/mL, about 670 ng·h/mL, about 680 ng·h/mL, about 690 ng·h/mL, about 700 ng·h/mL, about 710 ng·h/mL, about 720 ng·h/mL, about 730 ng·h/mL, about 740 ng·h/mL, about 750 ng·h/mL, about 760 ng·h/mL, about 770 ng·h/mL, about 780 ng·h/mL, about 790 ng·h/mL, about 800 ng·h/mL, about 810 ng·h/mL, about 820 ng·h/mL, about 830 ng·h/mL, about 840 ng·h/mL, about 850 ng·h/mL, about 860 ng·h/mL, about 870 ng·h/mL, about 880 ng·h/mL, about 890 ng·h/mL, about 900 ng·h/mL, about 910 ng·h/mL, about 920 ng·h/mL, about 930 ng·h/mL, about 940 ng·h/mL, about 950 ng·h/mL, about 960 ng·h/mL, about 970 ng·h/mL, about 980 ng·h/mL, about 990 ng·h/mL, about 1000 ng·h/mL, about 1010 ng·h/mL, about 1020 ng·h/mL, about 1030 ng·h/mL, about 1040 ng·h/mL, about 1050 ng·h/mL, about 1060 ng·h/mL, about 1070 ng·h/mL, about 1080 ng·h/mL, about 1090 ng·h/mL, about 1100 ng·h/mL, about 1110 ng·h/mL, about 1120 ng·h/mL, about 1130 ng·h/mL, about 1140 ng·h/mL, about 1150 ng·h/mL, about 1160 ng·h/mL, about 1170 ng·h/mL, about 1180 ng·h/mL, about 1190 ng·h/mL, to about 1200 ng·h/mL, including all values and subranges therebetween. In some embodiments, the present compositions provide an AUCₗₐₛₜ of about 50 ng·h/mL to about 250 ng·h/mL following a single administration of the composition. In some embodiments, the present compositions provide an AUCₗₐₛₜ of about 40 ng·h/mL to about 300 ng·h/mL, or about 150 ng·h/mL to about 250 ng·h/mL, or about 50 ng·h/mL to about 150 ng·h/mL following a single administration of the composition. In embodiments, the present compositions provide an AUCₗₐₛₜ (expressed in terms of ng*hr/mL) of about 20 ng·h/mL to about 500 ng·h/mL following a single administration of the composition. In embodiments, the present compositions are dosed in the fed state. In embodiments, the compositions are dosed in the fasted state.

In some embodiments, the present compositions provide steady state plasma Cmax levels of zolmitriptan that correlate to one or more statistically significant therapeutic effects when administered on a once-a-day, twice-a-day, or three times a day basis. In some embodiments, the therapeutically effective steady state plasma Cmax levels of zolmitriptan provided by the zolmitriptan compositions of the present disclosure range from about 0.05 ng/mL to about 100 ng/mL, including about 0.5 ng/mL, about 1 ng/mL, about 1.5 ng/mL, about 2 ng/mL, about 2.5 ng/mL, about 3 ng/mL, 4 ng/mL, 5 ng/mL, 6 ng/mL, 7 ng/mL, 8 ng/mL, 9 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL about 65 ng/mL, about 70 ng/mL, about 75 ng/mL, about 80 ng/mL, about 85 ng/mL, about 90 ng/mL, about 95 ng/ml, and about 100 ng/mL including all values and ranges there between. In some embodiments, the therapeutically effective steady state plasma Cmax levels of zolmitriptan provided by the zolmitriptan compositions of the present disclosure range from about 3 ng/mL to about 85 ng/mL. In embodiments, the present compositions are dosed in the fed state. In embodiments, the compositions are dosed in the fasted state.

In some embodiments, the present compositions provide a Cₘₐₓ of about 1 ng/mL to about 100 ng/mL following a single administration of the composition, including about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 38 ng/mL, about 39 ng/mL, about 40 ng/mL, 41 ng/mL, about 42 ng/mL, about 43 ng/mL, about 44 ng/mL, about 45 ng/mL, about 46 ng/mL, about 47 ng/mL, about 48 ng/mL, about 49 ng/mL, about 50 ng/mL, about 51 ng/mL, about 52 ng/mL, about 53 ng/mL, about 54 ng/mL, about 55 ng/mL, about 56 ng/mL, about 57 ng/mL, about 58 ng/mL, about 59 ng/mL, about 60 ng/mL, about 61 ng/mL, about 62 ng/mL, about 63 ng/mL, about 64 ng/mL, about 65 ng/mL, about 66 ng/mL, about 67 ng/mL, about 68 ng/mL, about 69 ng/mL, about 70 ng/mL, about 71 ng/mL, about 72 ng/mL, about 73 ng/mL, about 74 ng/mL, about 75 ng/mL, about 76 ng/mL, about 77 ng/mL, about 78 ng/mL, about 79 ng/mL, about 80 ng/mL, about 81 ng/mL, about 82 ng/mL, about 83 ng/mL, about 84 ng/mL, about 85 ng/mL, about 86 ng/mL, about 87 ng/mL, about 88 ng/mL, about 89 ng/mL, about 90 ng/mL, about 91 ng/mL, about 92 ng/mL, about 93 ng/mL, about 94 ng/mL, about 95 ng/mL, about 96 ng/mL, about 97 ng/mL, about 98 ng/mL, about 99 ng/mL, to about 100 ng/mL, including all values and subranges therebetween. In some embodiments, the present compositions provide a Cₘₐₓ of about 1 ng/mL to about 80 ng/mL. In some embodiments, the present compositions provide a Cₘₐₓ of about 3 ng/mL to about 30 ng/mL following a single administration of the composition. In embodiments, the present compositions provide a Cₘₐₓ of about 5 ng/mL to about 60 ng/mL following a single administration of the composition. In embodiments, the present compositions are dosed in the fed state. In embodiments, the compositions are dosed in the fasted state.

In some embodiments, the therapeutically effective steady state plasma Cmax levels of zolmitriptan is provided by administering one or more compositions of the present disclosure to provide a daily dose of zolmitriptan or a pharmaceutically acceptable salt thereof of between about 7.5 mg to about 150 mg. In some further embodiments, the therapeutically effective steady state plasma Cmax levels of zolmitriptan is provided by administering a composition of the present disclosure containing about 7.5 mg to about 150 mg of zolmitriptan or a pharmaceutically acceptable salt thereof once a day. In other further embodiments, the therapeutically effective steady state plasma Cmax levels of zolmitriptan is provided by administering a composition of the present disclosure containing between about 3.75 mg and 75 mg of zolmitriptan or a pharmaceutically acceptable salt thereof twice a day. In other further embodiments, the therapeutically effective steady state plasma Cmax levels of zolmitriptan is provided by administering a composition of the present disclosure containing between about 2.5 mg and 50 mg of zolmitriptan or a pharmaceutically acceptable salt thereof three times a day. In some embodiments, the therapeutically effective steady state plasma Cmax levels of zolmitriptan is provided by administering one or more compositions of the present disclosure to provide a daily dose of zolmitriptan or a pharmaceutically acceptable salt thereof of between about 12 mg to about 78 mg, including between about 12 mg, about 24 mg, about 36, mg, about 48 mg, to about 72 mg, including all values and subranges therebetween.

In some embodiments, the present compositions provide a t_{1/2} of about 1 hour to about 24 hours following a single administration of the composition, including about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, to about 24 hours, including all values and subranges therebetween. In some embodiments, the present compositions provide a tin of about 5 hours to about 18 hours following a single administration of the composition. In some embodiments, the present compositions provide a plasma half-life (tin) of about 8.5 hours to about 15 hours following a single administration of the composition.

In some embodiments, the present compositions provide a Tmax of about 1 hour to about 10 hours following a single administration of the composition, including about 1 hour, about 2 hours, about 3 hours, about 4 hours, about 5 hours, about 6 hours, about 7 hours, about 8 hours, about 9 hours, to about 10 hours, including all values and subranges therebetween. In some embodiments, the present compositions provide a Tmax of about 2 hours to about 5 hours following a single administration of the composition.

Appropriate pharmaceutical compositions of the present disclosure can be administered according to any clinically-acceptable route of administration of the composition to the subject. The manner in which the composition is administered is dependent, in part, upon the cause and/or location. One skilled in the art will recognize the advantages of certain routes of administration. The method includes administering an effective amount of the agent or compound (or composition comprising the agent or compound) to achieve a desired biological response, e.g., an amount effective to alleviate, ameliorate, or prevent, in whole or in part, a symptom of a condition to be treated, e.g., symptoms associated with autism spectrum disorder. In various aspects, the route of administration is systemic, e.g., oral or by injection. In some embodiments, zolmitriptan, or pharmaceutically acceptable salts thereof, is administered orally, nasally, transdermally, pulmonary, inhalationally, buccally, sublingually, intraperintoneally, subcutaneously, intramuscularly, intravenously, rectally, intrapleurally, intrathecally, intraportally, or parenterally. In some embodiments, the zolmitriptan is administered orally. In some embodiments, zolmitriptan is administered parenterally.

### Methods of Treating the Symptoms of ASD

In some embodiments, the present disclosure provides methods of treating the symptoms associated with autism spectrum disorder (ASD) comprising administering a zolmitriptan composition of the present disclosure. In some embodiments, the present methods comprise administering the zolmitriptan composition of the present disclosure as the sole composition used to treat the symptoms of ASD. In some embodiments, the present methods employ a zolmitriptan composition of the present disclosure in conjunction with one or more active ingredients used to treat the symptoms of ASD. In some embodiments, the zolmitriptan composition of the present disclosure is administered in combination with an additional active ingredient used to treat ASD, e.g., co-formulated or administered separately.

Non-limiting examples of ASD symptoms include irritability, difficulty with communication, difficulty with social interactions, obsessive interests, repetitive behaviors, inappropriate social interaction, poor eye contact, compulsive behavior, impulsivity, repetitive movements, self-harm, persistent repetition of words or actions learning disabilities, speech delays, intense interest in a limited number of things, problems paying attention, lack of awareness of others' emotions, depression, anxiety, changes in voice, sensitivity to sound, and tics. In some embodiments, the patient in need experiences a substantial decrease in irritability associated with ASD compared to prior to said treating. In some embodiments, the patient in need experiences a substantial improvement in sociability compared to prior to said treating.

In some embodiments, the present disclosure provides methods of treating the symptoms of ASD comprising administering an effective amount of a zolmitriptan composition of the present disclosure. In some embodiments, the zolmitriptan composition is administered as a monotherapy. In some embodiments, the zolmitriptan composition is administered as an adjunctive to the patient's existing therapy (e.g., the current standard of care). In some embodiments, the zolmitriptan composition is administered as an adjunctive to risperidone. In some embodiments, the zolmitriptan composition is administered as an adjunctive to aripiprazole.

In embodiments, the present disclosure provides methods of treating the irritability associated with autism comprising administering an effective amount of a zolmitriptan composition of the present disclosure. In embodiments, the present disclosure provides methods of treating the aggression associated with autism comprising administering an effective amount of a zolmitriptan composition of the present disclosure. In embodiments, the present disclosure provides methods of treating the lethargy associated with autism comprising administering an effective amount of a zolmitriptan composition of the present disclosure. In embodiments, the present disclosure provides methods of treating the sociability symptoms associated with autism comprising administering an effective amount of a zolmitriptan composition of the present disclosure to improve socialization. In embodiments, the present disclosure provides methods of reducing the social deficits associated with autism comprising administering an effective amount of a zolmitriptan composition of the present disclosure or a pharmaceutically acceptable salt thereof.

In some embodiments, after said treatment the patient experiences a substantial reduction of the symptoms of ASD that is characterized by improvement according to the Vineland Adaptive Behavior Scales, ADOS-2, CGI-C social deficit subscales, aberrant behavior checklist, the Autism Treatment Evaluation Checklist (ATEC), Social Responsiveness Scale, 2^{nd} Edition (SRS-2), or any combination thereof.

In embodiments, after said treatment the patient experiences an improvement in the symptoms of ASD compared to prior to the treatment that is characterized by an improvement according to the Autism Behavior Inventory (ABI)-Social Communication Domain Score.

The ABI-Social Communication Domain Score is a 62-item questionnaire for reporting the behaviors of subjects (ages: 3 years-adulthood) diagnosed with ASD. The tool is suitable for completion by parents or care/study partners of people with ASD. Each item assesses either quality (from not at all to without help) or frequency (never to very often) of a particular behavior. The Social Communication domain score is the sum of the scores in the social communication domain divided by the number of items in the domain.

In embodiments, after said treatment the patient experiences an improvement in symptoms associated with ASD compared to prior to the treatment that is characterized by about a 20% improvement, about a 25% improvement, about a 30% improvement, about a 35% improvement, about a 40% improvement, about a 45% improvement, about a 50% improvement, about a 55% improvement, about a 60% improvement, about a 65% improvement, about a 70% improvement, about a 80% improvement, about a 90% improvement, or about a 95% improvement in ABI-Social Communication Domain Score compared to prior to treatment.

In some embodiments, after said treatment the patient experiences an improvement in symptoms associated with ASD compared to prior to the treatment that is characterized by at least about a 20% improvement, at least about a 25% improvement, at least about a 30% improvement, at least about a 35% improvement, at least about a 40% improvement, at least about a 45% improvement, at least about a 50% improvement, at least about a 55% improvement, at least about a 60% improvement, at least about a 65% improvement, at least about a 70% improvement, at least about a 80% improvement, at least about a 90% improvement, or at least about a 95% improvement in ABI-Social Communication Domain Score compared to prior to treatment.

In embodiments, after said treatment the patient experiences an improvement in the symptoms of ASD compared to prior to the treatment that is characterized by an improvement according to the Clinician Global Impression of Improvement (CGI-I) score.

The CGI-I score is a single-item instrument based on a 7-point scale used to capture the Investigator's global impression of response. The Investigator or designee rates the improvement observed from 1-7 (1=very much improved; 2=much improved; 3=minimally improved; 4=no change; 5=minimally worse; 6=much worse; 7=very much worse).

In embodiments, after said treatment the patient experiences an improvement in symptoms associated with ASD compared to prior to the treatment and has a CGI-I score of about 1, about 2, about 3, about 4, or about 5 after treatment. In embodiments, after said treatment the patient experiences an improvement in symptoms associated with ASD and has a CGI-I score of about 1, about 2, or about 3. In embodiments, after said treatment the patient experiences an improvement in symptoms associated with ASD and has a CGI-I score of about 1 or 2.

In some embodiments, after said treatment the patient experiences an improvement in symptoms associated with ASD that is characterized by improvement according to the Autism Behavior Inventory-Clinician (ABI-C) Score compared to prior to the treatment.

The ABI-Clinician (ABI-C) captures the clinician rating of behaviors of a person with ASD that occurred over the week prior to assessment. It contains 14 items reflecting the core and associated autism behavior domains: Social Communication, Restrictive Behaviors, Mood and Anxiety, Self Regulation, and Challenging Behavior. Each item is rated on a 7-point scale from 1 (none; no symptoms present) to 7 (very severe; persistent interference with function or adaptation).

In some embodiments, the patient experiences an improvement in symptoms compared to prior to the treatment that is characterized by about a 20% improvement, about a 25% improvement, about a 30% improvement, about a 35% improvement, about a 40% improvement, about a 45% improvement, about a 50% improvement, about a 55% improvement, about a 60% improvement, about a 65% improvement, about a 70% improvement, about a 80% improvement, about a 90% improvement, or about a 95% improvement in ABI-C score compared to prior to treatment.

In some embodiments, the patient experiences an improvement in symptoms compared to prior to the treatment that is characterized by at least about a 20% improvement, at least about a 25% improvement, at least about a 30% improvement, at least about a 35% improvement, at least about a 40% improvement, at least about a 45% improvement, at least about a 50% improvement, at least about a 55% improvement, at least about a 60% improvement, at least about a 65% improvement, at least about a 70% improvement, at least about a 80% improvement, at least about a 90% improvement, or at least about a 95% improvement in ABI-C score compared to prior to treatment.

In some embodiments, after said treatment the patient experiences an improvement in the symptoms of ASD that is characterized by improvement according to the ABI Repetitive/Restrictive Behavior Domain Score compared to prior to the treatment.

Each item on the ABI-Repetitive/Restrictive Behavior Domain is rated by frequency (0=never to 3=very often). The domain score is the sum of all domain items scores divided by the number of items in the domain.

In some embodiments, after said treatment the patient experiences an improvement in the symptoms of ASD that is characterized by improvement according to the ABI Challenging Behavior Domain Score compared to prior to the treatment.

Each item on the ABI-Challenging Behavior Domain is rated by frequency (0=never to 3=very often). The domain score is the sum of all domain items scores divided by the number of items in the domain.

In some embodiments, after said treatment the patient experiences an improvement in the symptoms of ASD that is characterized by improvement according to the ABI-Short Form (ABI-S) Score compared to prior to the treatment.

The ABI-S is a 24-item short version of the ABI, containing items from each of the five domains. The domain score for each domain is calculated as the sum of scores all domain items divided by the number of items in the domain.

In some embodiments, after said treatment the patient experiences an improvement in the symptoms of ASD that is characterized by improvement according to the ABC-Social Withdrawal (ABC-SW) Subscale Score compared to prior to the treatment.

The ABC-SW subscale consists of 16 items of the ABC-2 rated from 0 (not a problem) to 3 (the problem is severe in degree).

In some embodiments, after said treatment the patient experiences an improvement in the symptoms of ASD that is characterized by improvement according to Vineland-3 (Domain Level Version) Score: total of Communication, Socialization, and Maladaptive behavior domains compared to prior to the treatment.

The Vineland-3 Domain Level Version contains 5 domains. Responses on each item are rated from 0 (never) to 2 (usually).

The Vineland Adaptive Behavior Scales (VABS), third edition, is a standardized measure of adaptive behavior used to evaluate the personal and social skills of an individual from birth through adulthood. Individuals can be evaluated on the VABS scale by either teachers or caregivers. According to the VABS, an individual is assigned a VABS adaptive behavior composite score, which measures an individual's functioning compare to others of his or her age. An individual is also assigned domain scores in communication, daily living skills, socialization, and motor skills to assess an individual's adaptive behavior strengths and weaknesses. An individual receives a score from 20 to 140 on each of the domain scores and the VABS adaptive behavior composite score. A score from 20 to 70 represents low adaptive level. A score from 71 to 85 represents moderately low adaptive level. A score from 86 to 114 represents moderately adequate adaptive level. A score from 115 to 129 represents moderately high adaptive level. A score from 130 to 140 represents high adaptive level.

In some embodiments, after said treatment the patient experiences a reduction of symptoms associated with ASD that is characterized by at least a one point increase in the VABS adaptive behavior composite score compared to prior to the treatment. In some embodiments, the increase in the VABS adaptive behavior score is about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, about 24 points, about 25 points, about 26 points, about 27 points, about 28 points, about 29 points, about 30 points, about 31 points, about 32 points, about 33 points, about 34 points, about 35 points, about 36 points, about 37 points, about 38 points, about 39 points, about 40 points, about 41 points, about 42 points, about 43 points, about 44 points, about 45 points, about 46 points, about 47 points, about 48 points, about 49 points, about 50 points, about 51 points, about 52 points, about 53 points, about 54 points, about 55 points, about 56 points, about 57 points, about 58 points, about 59 points, about 60 points, about 61 points, about 62 points, about 63 points, about 64 points, about 65 points, about 66 points, about 67 points, about 68 points, about 69 points, or about 70 points compared to prior to said treating.

In some embodiments, the increase in VABS adaptive behavior composite score is at least about 1%, at least about 5%, at least about 10%, at least about 15%, at least about 20%, at least about 25%, at least about 30%, at least about 35%, at least about 40%, at least about 45%, at least about 50%, at least about 55%, or at least about 60% compared to prior to said treating.

In some embodiments, after said treatment the patient experiences an increase in sociability that is characterized by an increase in the VABS adaptive behavior socialization domain score compared to prior to the treatment. In some embodiments, the increase in the VABS adaptive behavior socialization domain score is about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, about 24 points, about 25 points, about 26 points, about 27 points, about 28 points, about 29 points, about 30 points, about 31 points, about 32 points, about 33 points, about 34 points, about 35 points, about 36 points, about 37 points, about 38 points, about 39 points, about 40 points, about 41 points, about 42 points, about 43 points, about 44 points, about 45 points, about 46 points, about 47 points, about 48 points, about 49 points, about 50 points, about 51 points, about 52 points, about 53 points, about 54 points, about 55 points, about 56 points, about 57 points, about 58 points, about 59 points, about 60 points, about 61 points, about 62 points, about 63 points, about 64 points, about 65 points, about 66 points, about 67 points, about 68 points, about 69 points, or about 70 points compared to prior to said treating.

In some embodiments, the increase in VABS adaptive behavior socialization domain score is at least about 1%, at least about 5%, at least about 10%, at least about 15%, at least about 20%, at least about 25%, at least about 30%, at least about 35%, at least about 40%, at least about 45%, at least about 50%, at least about 55%, or at least about 60%, compared to prior to said treating. In some embodiments, the patient experiences an at least 10% improvement on the socialization domain score of VABS after treatment. In some embodiments, the patient experiences an at least 35% improvement on the socialization domain score of VABS after treatment.

In some embodiments, after said treatment the patient experiences an improvement in communication that is characterized by an increase in the VABS adaptive behavior communication domain score compared to prior to the treatment. In some embodiments, the increase in the VABS adaptive behavior communication domain is about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, about 24 points, about 25 points, about 26 points, about 27 points, about 28 points, about 29 points, about 30 points, about 31 points, about 32 points, about 33 points, about 34 points, about 35 points, about 36 points, about 37 points, about 38 points, about 39 points, about 40 points, about 41 points, about 42 points, about 43 points, about 44 points, about 45 points, about 46 points, about 47 points, about 48 points, about 49 points, about 50 points, about 51 points, about 52 points, about 53 points, about 54 points, about 55 points, about 56 points, about 57 points, about 58 points, about 59 points, about 60 points, about 61 points, about 62 points, about 63 points, about 64 points, about 65 points, about 66 points, about 67 points, about 68 points, about 69 points, or about 70 points, compared to prior to the treatment.

In some embodiments, the increase in VABS adaptive behavior communication domain score is at least about 1%, at least about 5%, at least about 10%, at least about 15%, at least about 20%, at least about 25%, at least about 30%, at least about 35%, at least about 40%, at least about 45%, at least about 50%, at least about 55%, or at least about 60% compared to prior to said treating.

The Autism Diagnostic Observation Schedule, Second Edition (ADOS-2) is an instrument that allows accurate assessment and diagnosis of ASD across age, developmental level, and language skills. The ADOS-2 is a clinician-administered observational assessment that comprises two behavioral domains: social affect (SA) and restricted and repetitive behaviors (RRB). An individual is administered one of the five ADOS-2 modules, which is selected on the basis of the individual's expressive language level and chronological age. The toddler module is for children between 12 and 30 months of age who do not consistently use phrase speech. Module 1 is for children 31 months and older who do not consistently use phrase speech. Module 2 is for children of any age who use phrase speech but are not verbally fluent. Module 3 is for verbally fluent children and young adolescents. Module 4 is for verbally fluent older adolescents and adults. An individual is assigned an ADOS-2 Composite Total score ranging from 1 to 10. Individuals with ASD are characterized by an ADOS-2 Composite Total score between 6 and 10.

In some embodiments, the patient experiences a reduction of symptoms associated with ASD associated with an at least one point decrease in the ADOS-2 Composite Total score compared to prior to said treating. In some embodiments, the patient experiences an improvement in the symptoms of ASD that is characterized by a decrease of at least 1 point, at least 2 points, at least 3 points, at least 4 points, or at least 5 points on the ADOS-2 Composite Total score compared to prior to said treating.

In some embodiments, the patient experiences an improvement in the symptoms of ASD that is characterized by an at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90% improvement in the ADOS-2 composite score compared to prior to said treating.

In some embodiments, the patient experiences a reduction of symptoms associated with ASD that is characterized by an at least one point decrease in the ADOS-2 module 4 social affect score compared to prior to said treatment. In some embodiments, the decrease in the ADOS-2 module 4 social affect score is correlated with an improvement in sociability. In some embodiments, the improvement in sociability is characterized by an at least 1 point, or at least 2 point, or at least 3 point, or at least 4 point, or at least 5 point, or at least 6 point decrease in the social affect score on the ADOS-2 module 4 compared to prior to said treating.

In some embodiments, the improvement in sociability is characterized by an at least 10%, or at least 15%, or at least 20%, or at least 25%, or at least 30%, or at least 35%, or at least 40%, or at least 45%, or at least 50%, or at least 55%, decrease in the social affect scale on ADOS-2 module 4 compared to prior to said treating. In some embodiments, the patient experiences an improvement in sociability that is characterized by an at least 10% decrease in the social reciprocity score on the Autism Diagnostic Observation Schedule module 4 compared to prior to said treating.

The clinical global impression-severity (CGI-S) scale is utilized by clinicians to rate the severity of an ASD patient's symptoms. An individual is assigned a score of 1 to 7. A score of 1 represents a normal patient. A score of 7 represents the score of a patient with ASD.

In some embodiments, after said treatment the patient experiences a reduction of symptoms associated with ASD that is characterized by at least a one point decrease in the CGI-S score compared to prior to said treating. In some embodiments, the patient experiences an at least 1 point, or at least 2 points, or at least 3 points, or at least 4 points, or at least 5 points decrease in the CGI-S scale compared to prior to said treating.

In some embodiments, the patient experiences an at least 5%, or at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90% reduction in the CGI-S scale compared to prior to said treating.

The clinical global impression-severity (CGI-C) scale is utilized by clinicians to evaluate a change in an ASD patient's symptoms. An individual is assigned a score from 1 (very much improved) to 7 (very much worse).

In some embodiments, after said treatment the patient experiences a reduction of symptoms associated with ASD that is characterized by at least a one point decrease in the CGI-C scale. In some embodiments, the patient experiences an improvement in irritability that is characterized by a CGI-C score of ≤1, ≤2, ≤3, or ≤4 after said treatment. In some embodiments, the patient's improvement in irritability that is characterized by a Clinical Global Impression-Change (CGI-C) score of ≤3 after said treatment. In some embodiments, the patient experiences an improvement in sociability that is characterized by a CGI-C score of ≤1, ≤2, ≤3, or ≤4 after said treatment. In some embodiments, the patient experiences an improvement in sociability that is characterized by a CGI-C score of <3 after said treatment.

The Autism Behavior Inventory (ABI) social deficit subscale is a measure used for assessing changes in the core and associated symptoms of ASD. An individual is administered the ABI-full (93 items) or ABI-short version (36) scales. An individual is scored from 0 points to 6 points on each item of the scales, where 0 is the absence of symptoms and 6 is maximum symptoms. An individual that receives a score of 0 does not exhibit symptoms. An individual that is assigned a score of 6 experiences severe ASD symptoms.

In some embodiments, the patient experiences an improvement in symptoms that is characterized by a decrease in ABI score of at least one point. In some embodiments, the patient experiences an improvement in symptoms that is characterized by a decrease in ABI score of at least 1 point, or at least 2 points, or at least 3 points, or at least 4 points, compared to prior to said treating.

In some embodiments, the patient experiences an improvement in symptoms that is characterized by an at least 5%, or at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95% reduction in ABI score compared to prior to said treating.

The childhood autism rating scale (CARS) is used to identify children two years and older with ASD. The CARS consists of 14 domains assessing behaviors associated with ASD, with a 15th domain rating general impressions of autism. Each domain is scored on a scale ranging from one to four; higher scores are associated with a higher level of impairment. Total scores can range from a low of 15 to a high of 60; scores below 30 indicate that the individual is in the non-autistic range, scores between 30 and 36.5 indicate mild to moderate autism, and scores from 37 to 60 indicate severe autism.

In some embodiments, after said treatment the patient experiences a reduction of symptoms associated with ASD that is characterized by a decrease in the CARS total score of about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, about 24 points, about 25 points, about 26 points, about 27 points, about 28 points, about 29 points, or about 30 points, compared to prior to said treating.

In some embodiments, after said treatment the patient experiences a reduction of symptoms associated with ASD that is characterized by a decrease in the CARS total score of at least 5%, or at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, compared to prior to said treating.

The social responsiveness scale, 2^{nd} Edition (SRS-2) is utilized to obtain an efficient quantitative measure of the various dimensions of interpersonal behavior, communication, and repetitive/stereotypic behavior associated with ASD. According to the SRS-2, an individual is assigned a proxy version total t-score, based on the SRS-2. An individual is assigned a proxy version t-score which reflects the sum of responses to 65 social responsiveness scale questions, which serves as an index of severity of social skills across the autism spectrum. An individual that receives a proxy version t-score of greater than 76 receives a severe clinical diagnosis of ASD. If an individual is assigned a proxy version t-score of between 66 and 75, which is associated with moderate deficiencies in reciprocal social behavior that are clinically significant and lead to a substantial interference in everyday social interactions. If the individual is assigned a proxy version t-score between 60 to 65, the individual exhibits mild to moderate deficits in social interaction. If a patient exhibits a proxy version t-score of 59 or below, the patient exhibits normal social interactions.

In some embodiments, prior to administering a zolmitriptan composition to a patient in need thereof, the patient in need thereof exhibits a proxy version total t-score of ≥66.

In some embodiments, the patient experiences an improvement in symptoms that is characterized by a decrease in proxy version total t-score of at least one point, or at least two points, or at least three points, or at least four points, or at least five points, or at least six points, or at least seven points, or at least eight points, or at least nine points, or at least ten points, or at least eleven points, or at least twelve points, or at least thirteen points, or at least fourteen points, or at least fifteen points, or at least sixteen points, or at least seventeen points, or at least eighteen points, or at least twenty points, or at least twenty one points, or at least twenty two points, or at least twenty three points, or at least twenty four points, or at least twenty five points, compared to prior to said treating. In some embodiments, the patient experiences an improvement in symptoms that is characterized by a decrease in proxy version total t-score of the SRS-2 of at least 5%, or at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, compared to prior to said treating.

The social responsiveness scale for adults (SRS-A) is a tool to evaluate social responsiveness in adulthood. The SRS-A contains 65 items which are scored from 0 to 3. If an individual receives a score of 0, the individual does not have the symptom. If an individual receives a score of 3, the individual has a severe symptom. An SRS-A total score of 67 indicates that an individual has ASD. The maximum SRS-A total score is 195.

In some embodiments, after said treatment the patient exhibits a reduction in symptoms associated with ASD that is characterized by a decrease in the SRS-A score of about 5%, about 10%, about 15%, about 20%, about 25%, about 30%, about 35%, about 40%, about 45%, about 50%, about 55%, about 60%, about 65%, about 70%, compared to prior to said treatment. In some embodiments, after said treatment the patient exhibits a reduction in symptoms associated with ASD that is characterized by an at least 10% decrease in the SRS-A score compared to prior to said treatment.

In some embodiments, an improvement in sociability is associated with an a decrease in the SRS-A score of about 10%, about 15%, about 20%, about 25%, about 30%, about 35%, about 40%, or about 50%, compared to prior to said treatment.

The aberrant behavior checklist (ABC) is a behavior rating scale that is utilized to rate individuals in five subscales: (1) irritability, agitation, and crying, (2) lethargy, social withdrawal, (3) stereotypic behavior, (4) hyperactivity and noncompliance, and (5) inappropriate speech. Individuals can be evaluated on the ABC by any adult that knows the individual well. The ABC contains a 58-item questionnaire that is utilized to assess the five-subscales. Each item on the 58-item questionnaire is rated from 0 to 3. A score of 0 means an absence in symptom. A score of 3 means an individual experiences the symptom with high severity. Items within each subscale are added to obtain a subscale score. Possible subscale scores on the ABC range from 0 to 48 with higher sub-scores indicating behavioral impairment.

In some embodiments, after said treatment the patient experiences a reduction of symptoms associated with ASD that is characterized by at least a one point decrease in the ABC irritability agitation and crying (ABC-I) sub-score. In some embodiments, after said treatment the patient experiences a reduction in irritability that is characterized by a decrease in the ABC-I communication domain score compared to prior to the treatment.

In some embodiments, the patient exhibits an ABC-I score of ≥18 prior to treatment.

In some embodiment, after said treatment the patient experiences a reduction in irritability that is characterized by an about 2 point, about 4 point, about 6 point, about 8 point, about 10 point, about 15 point, about 20 point, about 25 point, or about 30 point decrease in the ABC-I domain score compared to prior to said treatment. In some embodiments, the decrease in the ABC-I domain score is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% compared to prior to said treatment. In some embodiments, the decrease in the ABC-I domain score is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or about at least 95% compared to prior to said treatment.

In embodiments, after said treatment the patient experiences a reduction in irritability that is characterized by a 25% reduction on the irritability subscale and wherein the patient is rated as much improved or very much improved (i.e., 2 or 1, respectively) on the CGI improvement scale.

In some embodiments, after said treatment the patient experiences a reduction of symptoms associated with ASD that is characterized by at least a one point decrease in the ABC-Lethargy and Social Withdrawal (ABC-LSW) subscore compared to prior to said treatment. In some embodiments, after said treatment the patient experiences a reduction in lethargy and social withdrawal that is characterized by a decrease in the ABC-LSW communication domain score compared to prior to the treatment.

In some embodiments, the decrease in the ABC-LSW communication domain score is about 2 points, about 4 points, about 6 points, about 8 points, about 10 points, about 15 points, about 20 points, about 25 points, or about 30 points, compared to prior to said treatment.

In some embodiments, the decrease in the ABC-LSW communication domain score is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, compared to prior to said treatment.

The social communication questionnaire is a tool used by physicians to screen patients with ASD. An individual's caregiver rates the verbal individual on a score of 0 to 39 or the non-verbal individual on a scale of 0 to 33. An individual that has ASD is characterized by a social communication questionnaire that is above 15.

In some embodiments, after said treatment the patient experiences a reduction of symptoms associated with ASD that is characterized by an at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% decrease in the social communication questionnaire scale compared to prior to said treatment.

The pervasive developmental disorder behavior inventory (PDDBI) is utilized to evaluate the efficacy of the treatment of ASD. The PDDBI assesses both problem behaviors and appropriate social, language, and learning/memory skills. The PDDBI is divided into two behavioral dimensions: (a) approach-withdrawal problems, assessing maladaptive behaviors and (b) receptive/expressive social communication abilities, assessing social communication competence. The approach-withdrawal problem dimension is further divided into behavioral domains, including sensory/perceptual approach behaviors, ritualisms/resistance to chain (RITUAL), social pragmatic problems (SOCPP), semantic/pragmatic problems (SEMPP), arousal regulation problems (AROUSE), specific fears (FEARS), and aggressiveness (AGG) domains. The receptive/expressive social communication abilities, assessing social communication competence (REXSCA) is further divided into behavioral domains, including the social approach behaviors (SOCAPP), expressive language (EXPRESS), and learning memory and receptive language (LMRL) domains. An individual receives a T score for each domain and a composite score, which is the sum of the scores in each domain. An individual with ASD is assigned a T score of greater than 50.

In some embodiments, the pervasive developmental disorder behavior inventory (PDDBI) is utilized to characterize the reduction in symptoms associated with ASD provided by the methods of the disclosure. In some embodiments, a patient exhibits a T score of greater than 50 prior to said treatment. In some embodiments, after said treatment the patient experiences a reduction of symptoms associated with ASD that is characterized by a decrease in the PDDBI by about 5%, or about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95% compared to prior to said treating.

The ATEC is a scale utilized to evaluate the efficacy of ASD treatments. The ATEC is a one-page form designed to be completed by parents, teachers, or caretakers. The ATEC consists of four subtests: speech/language communication, sociability, sensory/cognitive awareness, and health/physical/behavior. An individual is assigned a speech/language communication subtest rating from 0 to 28. An individual is assigned a sociability subtest rating from 0 to 40. An individual is assigned a sensory/cognitive awareness subtest rating from 0 to 36. An individual is assigned a health/physical/behavior subtest rating of 0 to 75. The individual subsets are summed, and the individual can receive a maximum score of 180 points

In some embodiments, after said treatment the patient experiences a reduction of symptoms associated with ASD that is characterized by a decrease in the mean ATEC score compared to prior to the treatment. In some embodiments, after said treatment the patient experiences an improvement in speech/language communication that is characterized by a decrease in the speech/language communication subtest rating compared to prior to said treating. In some embodiments, after said treatment the patient experiences an improvement in sociability that is characterized by a decrease in the sociability subtest rating compared to prior to said treating. In some embodiments, after said treatment the patient experiences an improvement in sensory/cognitive awareness that is characterized by a decrease in sensory/cognitive awareness subtest rating compared to prior to said treating. In some embodiments, after said treatment the patient experiences an improvement in health/physical/behavior that is characterized by a decrease in health/physical/behavior rating, compared to prior to said treating.

In some embodiments, the patient exhibits a decrease on the sociability subsection of the ATEC of at least 1 point, or about 2 points, or about 3 points, or about 4 points, or about 5 points, or about 6 points, or about 7 points, or about 8 points, or about 9 points, or about 10 points, or about 11 points, or about 12 points, or about 13 points, or about 14 points, or about 15 points, or about 16 points, or about 17 points, or about 18 points, or about 19 points, or about 20 points compared to prior to said treating. In some embodiments, the patient exhibits a decrease on the sociability subsection of the ATEC of at least 1 point compared to prior to said treating.

In some embodiments, the patient exhibits a decrease on the sociability subsection of the ATEC of at least 5%, or at least 10%, or at least 15%, or at least 20%, or at least 25%, or at least 30%, or at least 35%, or at least 35%, or at least 40%, or at least 50% compared to prior to said treating. In some embodiments, the patient exhibits a decrease on the sociability subsection of the ATEC of at least 10% compared to prior to said treating.

Objective/performance-based assessments are utilized to measure the reduction in the symptoms associated with ASD after administering a therapeutically effective amount of a triptan to a patient in need thereof. In some embodiments, the objective/performance based assessments are selected from the group consisting of eye gaze tracking of social stimuli (eye tracking), parent engagement rating inventory (JERI), and Noldus Ethovision Analysis. In some embodiments, eye gaze tracking of social stimuli is utilized to characterize the reduction in symptoms associated with ASD provided by the methods of the disclosure. Patients with ASD exhibit significantly shorter look durations to the eyes (eye contact) as compared to their peers without ASD. Patients with ASD have more difficulty locating and processing pertinent social information the more rapidly stimuli are presented.

The PCIT provides training methods designed to help adults improve their parenting and language skills and to help children learn how to better control emotions. In some embodiments, the parent child interaction task (PCIT) is utilized to improve the relationship between the caregiver and the patient with ASD. In some embodiments, the PCIT is helpful for reducing child behavior issues and increasing communication and interaction skills within the family. In some embodiments, children who participate in CPT develop greater self-esteem, experience less anger and frustration, see an improvement in social, organizational, and play skills, feel safer and calmer, and communicate more effectively.

The Joint Engagement Ranking Inventory (JERI) is a tool which is utilized to measure targets for early invention for developmental disorders and delays, such as ASD. The JERI is an 18-item inventory developed to measure relevant intervention targets, such as unengagement, object engagement, joint engagement, stereotyped, restricted and repetitive behavior, attention to the caregiver, initiation of communication, expressive language level and use, scaffolding, following-in, caregiver affect, fluency and connectedness, and shared routines and rituals. An individual evaluated on the JERI scale receives a score from 1 to 7 on each item in the JERI inventory. A score of 7 is assigned to an individual with the most joint engagements. A score of 1 on an item is assigned to an individual that has no episodes of joint engagement. A lower JERI scale is correlated with ASD.

In some embodiments, a reduction in the symptoms in ASD is associated with lower scores on the JERI compared to prior to treatment by the methods of the disclosure. In some embodiments, after said treatment the patient experiences a reduction of symptoms associated with ASD that is characterized by a decrease in the JERI score compared to prior to the treatment. In some embodiments, the JERI scores are reduced by about 10%, about 20%, about 30%, about 40%, about 50%, about 60%, or about 70% compared to prior to the treatment.

The Ohio State University Autism Rating Scale (OARS-5) measures persistent impairment in social interactions, restrictive interests/activities and repetitions in behavior, and level of support from social, academic, and community (Hollway, J. A., Arnold, L. E., & Aman, M. G. (2017, September). OSU Autism Rating Scale-DSM-5 (OARS-5).). The OARS-5 was developed to provide three types of summary scores: (A) a Autism Symptom Count, based on clinical interview (OARS-5 Total Score); (b) a Weighted Mean Severity score based on severity of autism symptoms, derived from the clinical interview; (c) and an Impairment Index ranging from 0 to 9, based on the level of supports needed due to severity. The OARS-5 Total Score is equal to OARS-5 Social Deficits Subscale Score+OARS-5 Restricted Patterns of Interest Subscale Score.

The OARS-5 includes the following subscales:
Section A: Persistent impairment in social interactions across multiple settings (OARS-5 Social Deficits Subscale Score);
Section B: Restricted interests/activities and repetitive patterns of behavior (OARS-5 Restricted Patterns of Interest Subscale Score) and
Section C: Level of support for Section A (social interactions/communication) and B (restricted and repetitive behaviors).

In some embodiments, after said treatment the patient experiences a reduction of symptoms associated with ASD that is characterized by a decrease in the OARS-5 Total Score compared to prior to the treatment. In some embodiments, after said treatment the patient experiences a reduction of symptoms associated with ASD that is characterized by a decrease of about 1 point, about 2 points, about 3 points, or about 4 points, about 5 points, about 6 points, about 7 points, about 8 points, about 9 points or about 10 points in the OARS-5 Total Score compared to prior to the treatment.

In some embodiments, after said treatment the patient experiences a reduction of symptoms associated with ASD that is characterized by a decrease in the OARS-5 Social Deficits Subscale Score compared to prior to the treatment. In some embodiments, after said treatment the patient experiences a reduction of symptoms associated with ASD that is characterized by a decrease of about 1 point, about 2 points, about 3 points, or about 4 points, about 5 points, about 6 points, about 7 points, about 8 points or about 9 points in the OARS-5 Social Deficits Subscale Score compared to prior to the treatment.

In some embodiments, after said treatment the patient experiences a reduction of symptoms associated with ASD that is characterized by a decrease in the Ohio State University Autism Clinical Global Impression (OSU Autism CGI) total score compared to prior to the treatment. OSU Autism CGI-S total score is equal to OSU Autism CGI Severity Scale (OSU Autism CGI-S) Score+OSU Autism CGI-Improvement Scale (OSU Autism CGI-I) Score.

In some embodiments, after said treatment the patient experiences a reduction of symptoms associated with ASD that is characterized by a decrease of about 1 point, about 2 points, about 3 points, or about 4 points, about 5 points, about 6 points, or about 7 points in the OSU Autism CGI total score compared to prior to the treatment.

In some embodiments, after said treatment the patient experiences a reduction of symptoms associated with ASD that is characterized by a decrease in the Ohio State University Autism Clinical Global Impression-Improvement Scale (OSU Autism CGI-I) score compared to prior to the treatment. In some embodiments, after said treatment the patient experiences a reduction of symptoms associated with ASD that is characterized by a decrease of about 1 point, about 2 points, about 3 points, or about 4 points, about 5 points, about 6 points, or about 7 points in the OSU Autism CGI-I compared to prior to the treatment.

In some embodiments, after said treatment the patient experiences a reduction of symptoms associated with ASD that is characterized by a decrease in the Ohio State University Autism Clinical Global Impression-Severity Scale (OSU Autism CGI-S) score compared to prior to the treatment. In some embodiments, after said treatment the patient experiences a reduction of symptoms associated with ASD that is characterized by a decrease of about 1 point, about 2 points, about 3 points, or about 4 points, about 5 points, about 6 points, or about 7 points in the OSU Autism CGI-S compared to prior to the treatment.

### Patient Populations

In some embodiments, the present disclosure provides methods of treating the symptoms of ASD comprising administering an effective amount of a zolmitriptan composition of the present disclosure to a patient in need thereof.

In embodiments, the patient is an adolescent or adult with an autism spectrum disorder (ASD). In embodiments, the patient is at least 12 years old. In embodiments, the patient is 12-45 years old.

In some embodiments, the patient in need thereof is a patient diagnosed with ASD according to the DSM-5 diagnostic criteria.

In some embodiments, prior to treatment according to the methods of the disclosure, the ASD patient in need thereof has a full scale IQ score on the Weschsler Abbreviated Scale of Intelligence (WASI®)-II of ≥70.

In some embodiments, the present disclosure provides methods of treating the symptoms associated with ASD that are refractory to treatment with existing ASD treatments. In some embodiments, the symptoms associated with ASD are refractory to treatment with aripiprazole. In some embodiments, the symptoms associated with ASD are refractory to treatment with risperidone.

In some embodiments, the treated ASD patient exhibits atypical sensory processing. Sensory processing refers to the ability to register, process, and organize sensory information and to execute appropriate responses to environmental demands, which manifest as hypersensitivities or hyposensitivities to stimuli. The patient in need with atypical sensory processing may have aversions to the color, taste, smell, and/or texture of foods or medicines. In some embodiments, atypical sensory processing is manifested in the patient in need as non-compliance with taking medications. In some embodiments, the treated ASD patient refuses to swallow medications. In some embodiments, said medications are liquid formulations or pills.

In some embodiments, the treated ASD patient is an infant. In some embodiments, the treated ASD patient is a child. In some embodiments, the treated ASD patient is an adolescent. In some embodiments, the treated ASD patient is an adult. In some embodiments, the treated ASD patient is a geriatric patient.

### EXAMPLES

The present disclosure is further illustrated by reference to the following Examples. However, it is noted that these Examples, like the embodiments described above, are illustrative and are not to be construed as restricting the scope of the invention in any way.

### Example 1. Simulated Pharmacokinetics of Zolmitriptan Administration

Steady-state simulations of zolmitriptan human plasma concentration time data with various dosing regimens were performed using nonparametric superposition. The pharmacokinetic profile for nonparametric superposition was derived from median plasma zolmitriptan concentrations following a single oral dose of 2.5 mg to male healthy volunteers described in Seaber et al., The absolute bioavailability and effect of food on the pharmacokinetics of zolmitriptan in healthy volunteers., Br J Clin Pharmacol. 1998 November; 46(5):433-9 (Seaber). Steady-state simulated concentration-time data were analyzed using noncompartmental methods to determine C_{max,ss} and AUC_{0-24h,ss}. Pharmacokinetic parameters and nonparametric superposition were calculated using Phoenix WinNonlin version 8.1 (Certara Inc., Princeton, NJ, USA).

The approach of nonparametric superposition is used to predict drug concentrations after multiple dosing at steady-state and is based on non-compartmental results describing single dose data. The predictions are based upon the accumulation ratio derived from the terminal elimination rate constant. The basic assumptions for nonparametric superposition for a drug are that the average systemic clearance is constant, the rate and extent of absorption is the same for each dose, linear pharmacokinetics applies and that the pharmacokinetics of the drug after a single dose are not altered following subsequent doses.

The human pharmacokinetic profile of zolmitriptan was derived from the reported median plasma concentrations following a single oral dose of 2.5 mg to male healthy volunteers (Seaber, 1998).

The following table shows Simulated Steady-State Human Plasma Pharmacokinetic Parameters of Zolmitriptan following QD, BID and TID oral dosing regimens.

TABLE 1

| Dose | QD | | | BID | | | TID | | |
|---|---|---|---|---|---|---|---|---|---|
| (mg/ | AUC_{0-24 h, ss} | C_{max, ss} | C_{min, ss} | AUC_{0-24 h, ss} | C_{max, ss} | C_{min, ss} | AUC_{0-24 h, ss} | C_{max, ss} | C_{min, ss} |
| kg/ | (ng*h/ | (ng/ | (ng/ | (ng*h/ | (ng/ | (ng/ | (ng*h/ | (ng/ | (ng/ |
| dose) | mL) | mL) | mL) | mL) | mL) | mL) | mL) | mL) | mL) |
| 2.5 | 17.6 | 3.27 | 0.007 | 35.3 | 3.44 | 0.267 | 52.9 | 3.91 | 0.945 |
| 5 | 35.3 | 6.54 | 0.014 | 70.5 | 6.89 | 0.534 | 106 | 7.82 | 1.89 |
| 7.5 | 52.9 | 9.81 | 0.021 | 106 | 10.3 | 0.801 | 159 | 11.7 | 2.84 |
| 10 | 70.5 | 13.1 | 0.028 | 141 | 13.8 | 1.07 | 212 | 15.6 | 3.78 |
| 20 | 141 | 26.2 | 0.057 | 282 | 27.5 | 2.14 | 423 | 31.3 | 7.56 |
| 30 | 212 | 39.3 | 0.085 | 423 | 41.3 | 3.21 | 635 | 46.9 | 11.3 |
| 40 | 282 | 52.3 | 0.114 | 564 | 55.1 | 4.27 | 846 | 62.5 | 15.1 |
| 50 | 353 | 65.4 | 0.142 | 705 | 68.9 | 5.34 | 1058 | 78.2 | 18.9 |

QD: once a day dose
BID: twice a day dose (12 hours apart)
TID: three times a day (8 hours apart)
AUC_{0-24 h, ss}: area under concentration-time curve from time zero to 24 hours at steady-state
C_{max, ss}: maximum observed concentration at steady-state

### Example 2. In Vivo Evaluation of Zolmitriptan in a Mouse Model of Aggression

The rodent Resident-Intruder assay (RI) has been used to monitor aggressive behaviors related to the behavioral patterns exhibited in establishing and defending territory (Miczek et al., 1984). Correspondingly, the RI assay has been used preclinically to study the effect of drugs on rodent aggression (Miczek et al., 2001). The RI assay typically relies on the interpretation and scoring of offensive (resident animal) behavior patterns and may also include analysis of defensive (intruder animal) behavior patterns as well.

A CD-1-C57BL/6 combination was used to evaluate the effect of zolmitriptan (10 mg/kg) on aggressive behavior in CD-i mice using a rodent RI assay. Risperidone (0.3 mg/kg) was used as a comparator compound. Attack time was measured over a 5 min period as a cross over design.

Zolmitriptan dosed at 3 and 10 mg/kg (i.p.) produced Cmax values of 5.40 ng/mL and 34.42 ng/ml in CSF in adult male CD-1 mice, respectively. N-desmethyl zolmitriptan, (“NDMZ”, which is the primary metabolite of zolmitriptan found in humans) was below the lower limit of quantification in male CD-1 mice dosed at 3 and 10 mg/kg (i.p.).

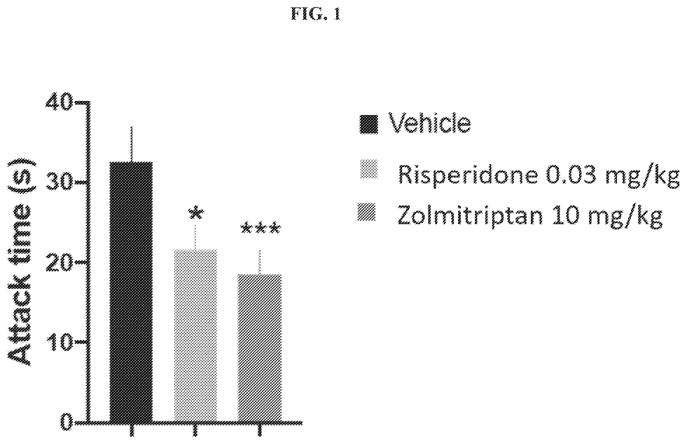

Results: As shown in FIG. 1 (and Table 2, below), CD-1 mice administered zolmitriptan at 10 mg/kg showed a greater reduction in attack time (s) compared to risperidone (0.03 mg/kg) and vehicle control.

TABLE 2

| | | Attack | | Latency to | |
|---|---|---|---|---|---|
| Treatment | N | Time (s) | SEM | Attack (s) | SEM |
| Vehicle | 25 | 32.65 | 4.219 | 4.705 | 1.524 |
| Risperidone 0.03 mg/kg | 25 | 21.58* | 3.123 | 12.98 | 4.18 |
| Zolmitriptan 10 mg/kg | 25 | 18.46*** | 3.028 | 13.54 | 5.520 |

### Example 3. In Vivo Evaluation of Zolmitriptan in a Mouse Model of ASD

The effect of zolmitriptan in a mouse model of valproic acid-induced autism spectrum disorder was investigated (Nicolini C et al., 2018 Exp. Neurol., 2018, 217-227; Bey A. L., and Jiang J. H, Current Protocols in Pharmacology, 1 Sep. 2014, 66:5.66.1-26). Specifically, sociability was evaluated in male c57/Bl6 mice that were previously exposed to valproic acid (VPA) via i.p. delivery of 500 mg/kg to their pregnant mothers at embryonic Day 13.

In this study, zolmitriptan was dissolved in 10% (2-Hydroxypropyl)-o-cyclodextrin in saline and administered intraperitoneally (i.p.) 15 minutes before mice were placed in the test chamber. Behavioral recordings were measured using an automated video tracking system (Noldus EthoVision v14) and analyzed and graphed using GraphPad Prism software v8. Sociability was assessed during a 10 min period in the 3-chamber Social Interaction Assay. The time spent (sec) in the social interaction zone (SIZ) in front of the juvenile novel C57/BL6 vs empty interaction zone (EIZ) were used to calculate the sociability scores for individual mice. Sociability index was calculated using SIZ/EIZ ratio. Social interaction preference was calculated using (SIZ)/(SIZ+EIZ)*100. Simultaneous recording up to 4 arenas were conducted for the social interaction assay. Paired and unpaired T tests were used as statistical tests.

Zolmitriptan dosed at 10 mg/kg (i.p.) produced a Cmax value of 17.83 ng/ml (15 min after administration) in the CSF of adult male c57/Bl6 mice (i.e., Tg2576 background strain and strain used for VPA-treatment). NDMZ was below the lower limit of quantification in the CSF of male c57/Bl6 mice dosed at 10 mg/kg (i.p.).

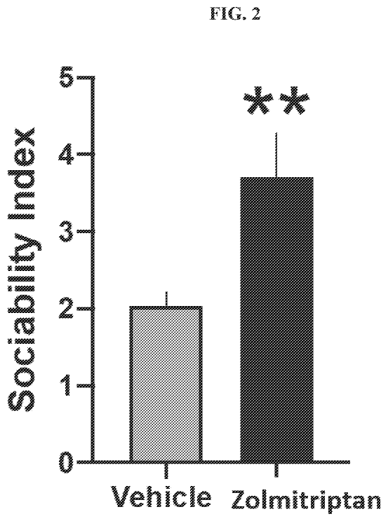

As shown in FIG. 2 zolmitriptan dosed at 10 mg/kg (i.p.) increased the sociability index in adult male c57/Bl6 mice previously exposed to valproic acid (VPA). The improvement in sociability of the zolmitriptan treated mice was statistically significant compared to the vehicle treated group.

### Example 4. Clinical Trial

An investigation of the safety, tolerability and pharmacokinetic response of oral zolmitriptan following multiple ascending doses in adult healthy volunteer subjects was undertaken.

Zolmitriptan 2.5 mg per tablet or placebo tablet was administered 3 times per day (TID) for an up-titration period, treatment period of 7 days, and a down-titration period. The dose regimen is depicted in Table 3.

TABLE 3

| | | Treatment | |
|---|---|---|---|
| Cohort | Up-titration | (7 days) | Down-titration |
| 1 | 2.5 mg TID (2 days) | 5.0 mg TID | 2.5 mg TID (2 days) |
| 2 | 2.5 mg TID (2 days), | 10.0 mg TID | 5.0 mg TID (2 days), |
| | 5.0 mg TID (2 days) | | 2.5 mg TID (2 days) |
| 3 | 5.0 mg TID (2 days), | 20.0 mg TID | 10.0 mg TID (2 days), |
| | 10.0 mg TID (2 days) | | 5.0 mg TID (2 days) |
| 4 | 5.0 mg TID (1 day), | 30.0 mg TID | 20.0 mg TID (2 days), |
| | 10.0 mg TID (2 days), | | 10.0 mg TID (2 days), |
| | 20.0 mg TID (2 days) | | 5.0 mg TID (1 day) |
| 5 | 5.0 mg TID (1 day), | 40.0 mg TID | 20.0 mg TID (2 days), |
| | 10.0 mg TID (2 days) | | 10.0 mg TID (2 days), |
| | 20.0 mg TID (2 days) | | 5.0 mg TID (1 day) |

A lumbar puncture was completed on each subject to determine the concentration of zolmitriptan in the CSF. CSF samples were collected on day 5 of treatment for Cohort 1 and day 7 of treatment for Cohorts 2 and 3, and day 8 of treatment for cohort 4 and 5. The CSF collection was 2 hours after the morning dose (+/−30 minutes). CSF concentration was measured using a validated bioanalytical method.

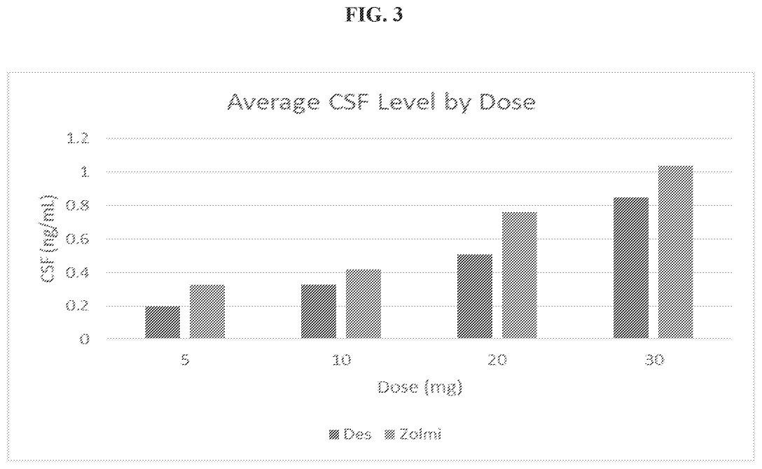

Results: As shown in FIG. 3, CSF levels measured in human exist in a ratio of zolmitriptan: NDMZ=1.0:0.75. Overall, Zolmitriptan was safe and well tolerated in all Cohorts.

### Example 5. Dose Studies

Human PK studies that correlate zolmitriptan oral dose administration to CSF concentrations (Example 4, i.e., mg zolmitriptan administered to CSF Cmax achieved), effective CSF concentrations determined from the valproate mouse model of ASD (Example 3) and species-specific binding assay data (human and mouse) were used to estimate effective human doses for treating the symptoms of ASD with zolmitriptan.

Specifically, 5-HT1b receptor occupancy and efficacy models measured potency of zolmitriptan and NMDZ at the 5-HT1b receptor and the ratio of zolmitriptan: NDMZ in human CSF. The 5-HT1b receptor efficacy model was used to predict the level of 5-HT1b activation (i.e., efficacy) by both zolmitriptan and NDMZ based on the concentration of zolmitriptan in the CSF. From this target zolmitriptan dose ranges required to achieve an effective CSF exposures in humans were calculated.

### 35S-GTPγS Binding Assay

In this study in vitro binding assays were conducted to measure the affinity of zolmitriptan and its active metabolite NDMZ to rat and human 5-HT1B receptors.

Zolmitriptan was tested in a SPA-based 35S-GTPγS binding assay in cells expressing human 5-HT1b (h5-HT1b) receptors or rat recombinant 5-HT1b (r5-HT1b) receptors at ten concentrations in duplicate. The EC50 vales are shown in Table 4.

TABLE 4
Functional Assay

| Triptan | h5-HT1b EC50 (nM) | r5-HT1b EC50 (nM) |
|---|---|---|
| Zolmitriptan | 4 | 33 |

Zolmitriptan exhibited lower apparent affinity (i.e., EC50) at rat 5-HT1b receptors than human 5-HT1b receptors. In the case of zolmitriptan, the difference between species is 8.3-fold.

Mouse and rat orthologs of 5-HT1b are 98% identical. They differ by only two amino acids, both of which are conservative changes and are removed from the agonist binding pocket (i.e., mouse/rat E152D in intracellular loop 1 and mouse/rat M192V in extracellular loop 2).

Based on this data, the CSF levels required for zolmitriptan to provide a therapeutic effect in humans is about 8.3 fold lower than the CSF levels required to provide similar activity in mice.

Assuming that CSF values mirror the free concentration of drugs in the brain, the estimated effective Cmax CSF values can be expressed as:

CD-1 (mouse aggression model, Example 2); 3 mg/Kg (i.p.); CSF Cmax=18.8 nM or EC36 (36% activity in GTPgS).

c57/Bl6 (ASD mouse model, Example 3); 10 mg/Kg (i.p.); CSF Cmax=62 nM EC65 (65% activity in GTPgS).

### Radioligand Binding Competition Assay

Zolmitriptan and NDMZ (the active metabolite of Zolmitriptan in humans) were evaluated for affinity for the human 5-HT1B receptor using a radioligand binding competition assay with 3H-5-CT and recombinant human 5-HT1B receptor at ten concentrations in duplicate. The affinity of the test compounds to the h5-HT1b receptor is shown in Table 5. The Ki values were derived from IC50 values using Cheng-Prusoff equation.

TABLE 5
Binding Assay

| Test compound | Ki (nM) |
|---|---|
| Zolmitriptan | 3.34 |
| NDMZ | 1.18 |

### 5-HT1b Receptor Occupancy and Efficacy Models

Using classical receptor theory, the receptor occupancy when considering two ligands (i.e., entities) can be predicted using the following relationship:

Total % occupancy=(% occupancy by A)+{[100%−(% occupancy by A)]×(% occupancy by B)} when considering a fixed concentration of both A and B.

Given that the ratio of zolmitriptan: NDMZ in humans is 1.0:0.75 in human CSF (FIG. 3) and that NDMZ is 2.83-fold more potent than zolmitriptan, a model of 5-HT1b occupancy was constructed using the Total % occupancy formula above as the basis. The model was converted to predict the level of 5-HT1b activation (i.e., efficacy) by both zolmitriptan and NDMZ based on the concentration of zolmitriptan in the CSF by substituting Occupancy for Efficacy as predicted by the Clark equation (Efficacy=[L]/([L]+Ki); [L]=ligand concentration) for both zolmitriptan and NDMZ:

Total % Efficacy={[Zolmitriptan]/([Zolmitriptan]+Ki,Z)}+(100−{[Zolmitriptan]/([Zolmitriptan]+Ki,Z)}×{[NDMZ]/([NDMZ]+Ki,N)}

Using the measured EC50 value for Zolmitriptan in the GTPgS assay NMDZ's 2.83-fold greater affinity for the human 5-HT1b receptor, and the measured relationship of Zolmitriptan: NDMZ=1.0:0.75, Total % Efficacy was calculated for a range of zolmitriptan concentrations and the resulting predicted Total % Efficacy was fitted with a logistic equation:

Total % Efficacy=100/(1+10\hat{ }((Log EC50−[Zolmitriptan]))) and Log EC50 is calculated to be −8.963. The concentration of zolmitriptan in human CSF predicted to induce 50% efficacy (i.e., EC50) of 5-HT1b by zolmitriptan and NDMZ is 1.09 nM or 0.31 ng/ml.

The concentration of zolmitriptan required to achieve 50% Total Efficacy is 3.7-fold that of the zolmitriptan EC50 because of the combined effect of the potent metabolite.

Zolmitriptan Human Dose Range The dose required to achieve the predicted effective CSF concentration range in humans occurs across a range of 5-HT1b activity shown in Table 6.

TABLE 6

| | | Zolmitriptan human dose |
|---|---|---|
| Activity | Zolmitriptan [CSF] | (mg) |
| EC20-EC80 | 0.078-1.24 ng/ml | 1.25-35 |
| EC25-EC75 | 0.103-0.93 ng/ml | 1.7-30 |
| EC30-EC70 | 0.135-0.713 ng/ml | 2.25-20 |
| EC35-EC65 | 0.167-0.577 ng/ml | 3-15 |
| EC40-EC60 | 0.207-0.465 ng/ml | 5-12.5 |

### Example 6. Manufacture of Zolmitriptan Tablets

Zolmitriptan drug substance was jet-milled to produce a micronized drug substance dry powder.

Immediate Release Layer Blend Compounding: Micronized Zolmitriptan and the excipients disclosed in the Table below were weighed and sieved for blending. Micronized Zolmitriptan, anhydrous lactose (such as Duralac® H), Microcrystalline Cellulose (such as Avicel® PH102), Sodium Starch Glycolate (such as Explotab), Colloidal Silicon Dioxide (such as Cab-O-Sil® M5P), and Magnesium Stearate (such as Hyqual® Vegetable Source), were mixed and blended in a diffusional blender. The blender was discharged and the blend was stored, in polyethylene bags inside HDPE pails at room temperature, for subsequent tableting operations.

Extended-Release Layer Blend Compounding: Micronized Zolmitriptan, polyethylene oxide (such as Polyox™ WSR-303 LEO) and the other excipients disclosed in the Table below were weighed and sieved for blending. Micronized Zolmitriptan, polyethylene oxide (such as Polyox™ WSR-303 LEO), Microcrystalline Cellulose (such as Ceolusrm KG-802), and Magnesium Stearate (such as Hyqual® Vegetable Source) were mixed and blended in a diffusional blender. The blender was discharged and the blend was stored, in polyethylene bags inside HDPE pails at room temperature, for subsequent tableting operations.

Tableting: The ER layer blend and IR layer blend were compressed using a Bilayer tablet Press to manufacture tablets.

TABLE 7
Zolmitriptan 3 mg IR/9 mg ER Oral Tablets

| | Composition | |
|---|---|---|
| Component | mg/tablet | % w/w |

IR LAYER

| | | |
|---|---|---|
| Zolmitriptan, Micronized | 3.0 | 0.56 |
| Lactose, Anhydrous (Duralac ® H) | 98.05 | 18.16 |
| Microcrystalline Cellulose (Avicel ® PH102) | 31.6 | 5.85 |
| Sodium Starch Glycolate (Explotab ®) | 5.6 | 1.04 |
| Colloidal Silicon Dioxide (Cab-O-Sil ® M5P) | 0.7 | 0.13 |
| Magnesium Stearate (Hyqual ® Vegetable Source) | 1.05 | 0.19 |
| IR Layer Total | 140.0 | 25.9 |

GASTRIC RETENTIVE ER LAYER

| | | |
|---|---|---|
| Zolmitriptan, Micronized | 9.0 | 1.67 |
| Polyethylene Oxide (Polyox ™ WSR-303 LEO) | 240.0 | 44.44 |
| Microcrystalline Cellulose (Ceolus ™ KG802) | 149.0 | 27.59 |
| Magnesium Stearate (Hyqual ® Vegetable Source) | 2.0 | 0.37 |
| ER Layer Total | 400.0 | 74.1 |
| Tablet Total | 540.0 | 100.0 |

TABLE 8
Zolmitriptan 6 mg IR/18 mg ER Oral Tablets

| | Composition | |
|---|---|---|
| Component | mg/tablet | % w/w |

IR LAYER

| | | |
|---|---|---|
| Zolmitriptan, Micronized | 6.0 | 1.11 |
| Lactose, Anhydrous (Duralac ® H) | 95.8 | 17.74 |
| Microcrystalline Cellulose (Ceolus ™ KG802) | 30.85 | 5.71 |
| Sodium Starch Glycolate (Explotab ®) | 5.6 | 1.04 |
| Colloidal Silicon Dioxide (Cab-O-Sil ® M5P) | 0.7 | 0.13 |
| Magnesium Stearate (Hyqual ® Vegetable Source) | 1.05 | 0.19 |
| IR Layer Total | 140.0 | 25.9 |

GASTRIC RETENTIVE ER LAYER

| | | |
|---|---|---|
| Zolmitriptan, Micronized | 18.0 | 3.33 |
| Polyethylene Oxide (Polyox ™ WSR-303 LEO) | 240.0 | 44.44 |
| Microcrystalline Cellulose (Ceolus ™ KG802) | 140.0 | 25.93 |
| Magnesium Stearate (Hyqual ® Vegetable Source) | 2.0 | 0.37 |
| ER Layer Total | 400.0 | 74.1 |
| Tablet Total | 540.0 | 100.0 |

### Example 7. Dissolution Measurements

Dissolution testing was performed as described in Table 9 and assessed using a Pion Rainbow Dissolution Monitoring System. The Pion was set to collect spectra for quantitation every 5 minutes for the first hour followed by every 20 minutes for the next 17 hours.

TABLE 9
Dissolution Testing Conditions

| Dissolution Parameter | Value | |
|---|---|---|
| Dissolution Apparatus | USP Apparatus II (Paddles) | |
| Dissolution Vessel | Standard USP 1000 mL | |
| Paddle Speed | 50 | RPM |
| Dissolution Medium | 0.1N HCl | |
| Medium Volume | 900 | mL |
| Medium Temperature | 37° | C. |

| | |
|---|---|
| Number of Units Tested | 3 (introduced to vessels in wire sinkers) |

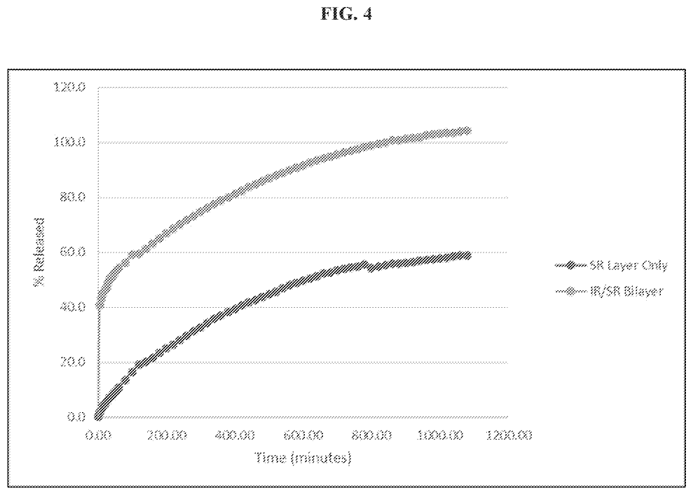

FIG. 4 provides a comparison of dissolution profiles for 15 mg zolmitriptan extended release tablets and 25 mg zolmitriptan immediate release/extended release oral bilayer tablets (10 mg IR zolmitriptan/15 mg ER zolmitriptan). As shown in FIG. 4 nearly complete zolmitriptan release from the IR layer is seen by the 5-minute time point. The zolmitriptan release remaining in the ER layer of the bilayer tablet matched the profile of the ER layer only tablet.

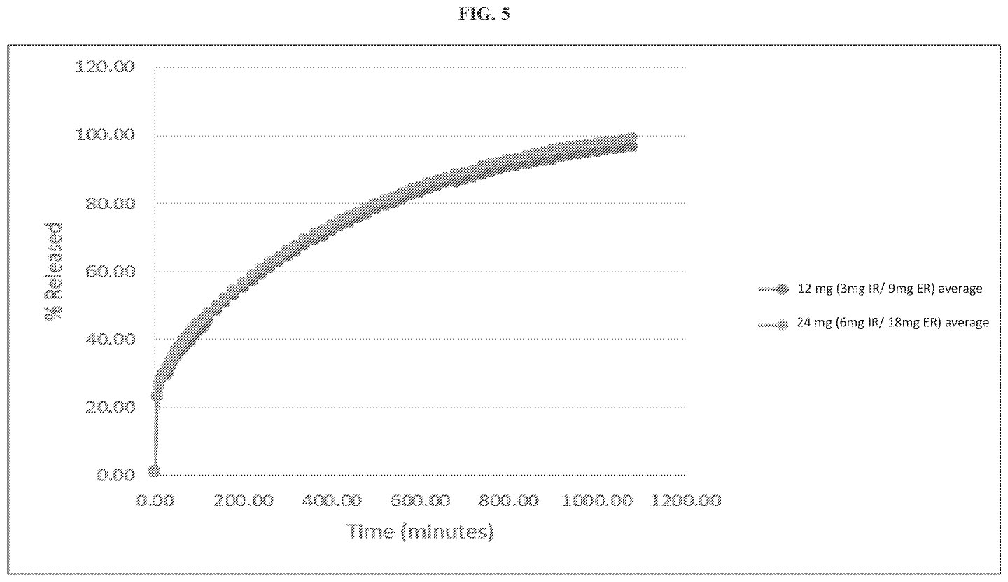

The dissolution curves for 12 mg (3 mg IR/9 mg ER) and 24 mg (6 mg IR/18 mg ER) zolmitriptan IR/ER oral bilayer tablets described in Example 6 are shown in FIG. 5.

### Example 8. Simulated Pharmacokinetics for Zolmitriptan IR/ER Oral Bilayer Tablets Under Fasted and Fed Conditions

Physiologically-based pharmacokinetic (PBPK) modeling and simulations were applied to facilitate development of zolmitriptan bilayer tablet formulation, a combination of immediate-release (IR) and extended-release (ER) gastroretentive components. The PK profiles for Zolmitriptan 12 mg (3 mg IR/9 mg ER) and 24 mg (6 mg IR/18 mg ER) tablets upon oral once-daily (QD) administration were projected using the refined PBPK GP 9.7 model and in vitro dissolution data (USP Apparatus 2, 50 RPM, 900 mL 01N HCl) as input. The gastroretention times (GRT) of 2 hours and 8 hours were specified for fasted and fed light meal and fed heavy meal conditions, respectively. The projections under different meal conditions are compared in the Table 10 below.

TABLE 10
Projected Pharmacokinetics for Zolmitriptan 12
mg (3 mg IR/9 mg ER) and 24 mg (6 mg IR/18 mg
ER) Oral Tablets under Fasted and Fed Conditions.

| | | Cₘₐₓ | AUC_{0-24 h} |
|---|---|---|---|
| Dose | Conditions | (ng/mL) | (ng × h/mL) |
| 12 mg (3 mg | Fasted (2 h GRT) | 11 | 87 |
| IR/9 mg ER) | Fed-Light (8 h GRT) | 10 | 106 |
| | Fed-FDA (8 h GRT) | 9.9 | 103 |
| 24 mg (6 mg | Fed Default | 19 | 206 |
| IR/18 mg ER) | | | |

### Example 9. Open-Label Cross-Over Relative Bioavailability Pharmacokinetic Study

This study was an open-label cross-over relative bioavailability pharmacokinetic study that evaluated a zolmitriptan bi-layer immediate-release/extended-release gastroretentive 24 mg oral tablet formulation (6 mg immediate-release/18 mg extended-release) described in Example 6 in a total of 12 healthy adult volunteer subjects under fasted and fed conditions. All subjects were domiciled at the study facility until discharge on Day 6, with telephonic follow-up Day 11 and Day 18. The dosing regimen included:
Day 1: zolmitriptan immediate-release 20 mg in the morning under fasted conditions
Day 2: Zolmitriptan bi-layer immediate-release/extended-release gastroretentive 24 mg oral tablet (6 mg IR/18 mg ER) in the morning under fasted conditions
Day 4: Zolmitriptan bi-layer immediate-release/extended-release gastroretentive 24 mg oral tablet (6 mg IR/18 mg ER) in the morning under fed conditions

All subjects completed the study and no subjects discontinued study treatment due to adverse events.

A summary of preliminary pharmacokinetic parameters for the 12 study subjects is presented in Table 11 for Day 1 zolmitriptan immediate-release dosing in fasted state, Day 2-24 mg zolmitriptan bilayer tablet dosing in the fasted state, and Day 4—Zolmitriptan bi-layer immediate-release/extended-release gastroretentive 24 mg oral tablet dosing in the fed state.

TABLE 11
Pharmacokinetic Summary Data Study

| | | | | | |
|---|---|---|---|---|---|
| T½ | Tₘₐₓ | Cₘₐₓ | C_{max—}D | AUCₗₐₛₜ | AUC_{last—}D |
| (h) | (h) | (ng/mL) | (ng/mL/mg) | (h*ng/ml) | (h*ng/mL/mg) |

Zolmitriptan Immediate-release 20 mg (mean value) (N = 12)

| | | | | | |
|---|---|---|---|---|---|
| 4.7 | 2.3 | 33.1 | 1.7 | 200 | 10.0 |

Zolmitriptan Bilayer Formulation 24 mg (6 mg IR/18 mg ER)
Fasted state (mean value) (N = 12)

| | | | | | |
|---|---|---|---|---|---|
| 15.2 | 4.4 | 19.4 | 0.8 | 170 | 7.1 |

Zolmitriptan Bilayer Formulation 24 mg (6 mg IR/18 mg ER)
Fed state (mean value) (N = 12)

| | | | | | |
|---|---|---|---|---|---|
| 8.5 | 4.9 | 15.3 | 0.6 | 187 | 7.8 |

Plasma concentrations for zolmitriptan are summarized in Table 12.

TABLE 12
Plasma Concentrations following administration
of 24 mg zolmitriptan bilayer tablet
Zolmitriptan (ng/mL) Plasma Concentrations

| | | | | |
|---|---|---|---|---|
| Time | n | Mean | Minimum | Maximum |

Day 2 Zolmitriptan bi-layer immediate-release/extended-release
gastroretentive oral tablet dosing 24 mg (fasted)

| | | | | |
|---|---|---|---|---|
| Hour −1 | 12 | 0.8699 | 0.0000 | 2.4644 |
| Hour 2 | 12 | 12.9958 | 6.7267 | 24.6517 |
| Hour 4 | 12 | 14.7420 | 6.2008 | 22.9479 |
| Hour 5 | 12 | 17.7949 | 4.7501 | 38.1102 |
| Hour 6 | 12 | 13.6712 | 2.7428 | 31.4936 |
| Hour 8 | 12 | 8.9947 | 2.3197 | 21.5604 |
| Hour 10 | 11 | 5.7414 | 1.5281 | 18.7824 |
| Hour 12 | 12 | 4.4472 | 1.2861 | 16.5388 |
| Hour 14 | 12 | 3.3930 | 1.0768 | 13.2155 |
| Hour 16 | 12 | 2.6760 | 1.0538 | 11.1437 |
| Hour 24 | 12 | 1.1938 | 0.5075 | 3.6175 |

Day 4 Zolmitriptan bi-layer immediate-release/extended-release
gastroretentive oral tablet dosing 24 mg (fed)

| | | | | |
|---|---|---|---|---|
| Hour −1 | 12 | 0.3778 | 0.0000 | 1.0406 |
| Hour 2 | 12 | 10.8128 | 2.3616 | 21.7767 |
| Hour 4 | 12 | 12.7625 | 7.1069 | 23.2936 |
| Hour 5 | 12 | 15.0586 | 7.3398 | 26.2345 |
| Hour 6 | 12 | 12.1702 | 4.7436 | 20.7219 |
| Hour 8 | 12 | 10.8649 | 4.4541 | 15.8481 |
| Hour 10 | 12 | 9.9559 | 4.4771 | 19.6035 |
| Hour 12 | 12 | 7.1100 | 3.3948 | 14.9441 |
| Hour 14 | 12 | 5.2781 | 1.8575 | 11.3851 |
| Hour 16 | 12 | 3.7720 | 1.0202 | 9.1381 |
| Hour 24 | 12 | 2.1379 | 0.5793 | 4.7834 |

TABLE 13
Blood Serotonin Levels Study of
24 mg zolmitriptan bilayer tablet

| Serotonin (nmol/L) | | | | | | Change from | | |
|---|---|---|---|---|---|---|---|---|
| | Study | | Values | | | Study Baseline¹ | | |
| Study Day | Hour | N | Mean | Min | Max | Mean | Min | Max |
| Day 1 | Hour −1 | 12 | 577.3 | 223 | 851 | | | |
| Dosing: | Hour 2 | 11 | 608.6 | 295 | 879 | 18.64 | −139 | 245 |
| Zolmitriptan | | | | | | | | |
| immediate- | | | | | | | | |
| release | | | | | | | | |
| 20 mg (fasted) | | | | | | | | |
| Day 2-3 | Hour −1 | 12 | 588.9 | 295 | 814 | 11.67 | −145 | 186 |
| Dosing: | Hour 2 | 12 | 520.3 | 261 | 827 | −56.9 | −409 | 193 |
| Day 2— | | | | | | | | |
| Zolmitriptan | | | | | | | | |
| bi-layer | | | | | | | | |
| immediate- | | | | | | | | |
| release/ | | | | | | | | |
| extended- | | | | | | | | |
| release | | | | | | | | |
| gastroretentive | | | | | | | | |
| oral tablet | | | | | | | | |
| 24 mg (fasted) | | | | | | | | |
| Day 4 to | Hour −1 | 12 | 571.6 | 320 | 778 | −5.67 | −173 | 144 |
| Day 5 | Hour 2 | 12 | 601.2 | 260 | 879 | 23.92 | −94 | 245 |
| Dosing: | | | | | | | | |
| Day 4— | | | | | | | | |
| Zolmitriptan | | | | | | | | |
| bi-layer | | | | | | | | |
| immediate- | | | | | | | | |
| release/ | | | | | | | | |
| extended- | | | | | | | | |
| release | | | | | | | | |
| gastroretentive | | | | | | | | |
| oral tablet | | | | | | | | |
| 24 mg (fed) | | | | | | | | |

### Example 10. Multi-Center, Randomized, Double-Blind, Placebo-Controlled Study

This study is a Phase 2, multi-center, randomized, double-blind, placebo-controlled study that will enroll approximately 150 adolescent and adult subjects with ASD. The primary objective of the study will be to evaluate the efficacy of zolmitriptan bilayer immediate-release (IR)/extended-release (ER, gastroretentive) oral tablet formulation compared with placebo for the treatment of care/study partner-reported social communication deficits in patients with ASD. Subjects will be randomized to study treatment in a 1:1 ratio of zolmitriptan bilayer immediate-release (IR)/extended-release (ER, gastroretentive) oral tablet formulation: placebo.

Dose/Administration: zolmitriptan bilayer immediate-release (IR)/extended-release (ER, gastroretentive) oral tablet formulation is taken orally once daily in the morning with food, and provided as a tablet in two dose strengths of 12 mg (3 mg IR/9 mg ER) and 24 mg tablet (6 mg IR/18 mg ER). Dose levels for this study are 12 mg (provided as one 12 mg).

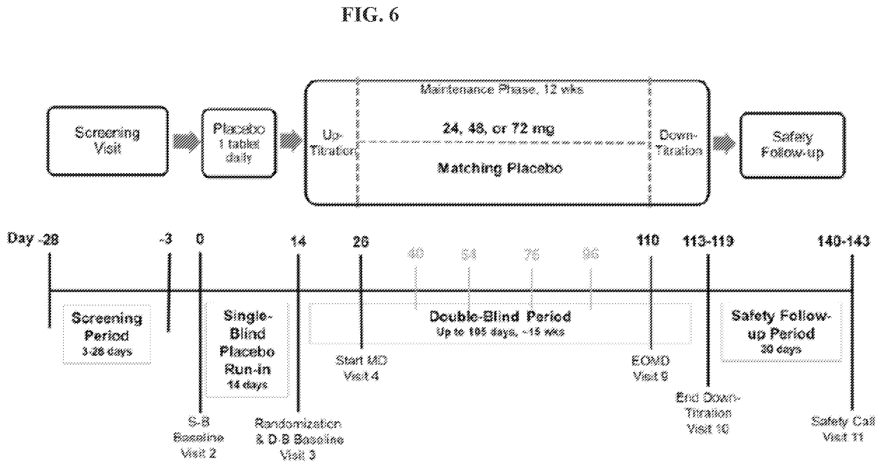

The study is design is shown in FIG. 6. Treatment will begin with 2 weeks of once daily dosing of zolmitriptan bilayer immediate-release (IR)/extended-release (ER, gastroretentive) oral tablet formulation or placebo followed by a 9-12 day dose-titration phase until the maximum allowable tolerated dose (based on subject weight) is reached. For example, dose escalation will occur every 3 days as follows: 12 mg (starting dose), 24 mg, 48 mg, 72 mg, or matching placebo to reach a goal dose (not to exceed) of either 48 mg (if screening weight ≤55 kg OR if female taking oral contraceptives) or 72 mg (if screening weight >55 kg AND female not taking oral contraceptives). If escalation doses cannot be tolerated, the subject or can reduce to last tolerated dose, if that dose is 24 mg or 48 mg.

The maximum dose tolerated during dose titration will become the maintenance dose (MD) for that subject, and the subject will remain on this dose for 12 weeks, followed by a down titration. An independent DSMC will monitor trial progress and ensure that the safety of trial subjects is not compromised.

Zolmitriptan bilayer immediate-release (IR)/extended-release (ER, gastroretentive) oral tablet formulation is taken orally once daily for up to 16 weeks and provided as a tablet in two dose strengths of 12 mg (3 mg IR/9 mg ER) and 24 mg tablet (6 mg IR/18 mg ER). Dose levels for this study are 12 mg (provided as one 12 mg tablet), 24 mg (one 24 mg tablet), 48 mg (two 24 mg tablets), and 72 mg (three 24 mg tablets).

### Primary Outcome Measures:

Change from Baseline in Autism Behavior Inventory (ABI)-Social Communication Domain Score [Time Frame: Baseline Up to Day 110]

Change from baseline in the ABI-Social Communication Domain Score will be reported. The ABI is a 62-item questionnaire for reporting the behaviors of subjects (ages: 3 years-adulthood) diagnosed with ASD. The tool is suitable for completion by parents or care/study partners of people with ASD. Each item assesses either quality (from not at all to without help) or frequency (never to very often) of a particular behavior. The Social Communication domain score is the sum of the scores in the social communication domain divided by the number of items in the domain.

### Secondary Outcome Measures:

Change from Baseline in Clinician Global Impression of Improvement (CGI-I) [Time Frame: Day 110]

The CGI-I score is a single-item instrument based on a 7-point scale routinely used in clinical trials to capture the Investigator's global impression of response. The Investigator or designee rates the improvement observed from 1 (very much improved) to 7 (very much worse).

Change from Baseline in Autism Behavior Inventory-Clinician (ABI-C) Score [Time Frame: Baseline Up to Day 110]

The ABI-Clinician (ABI-C) captures the clinician rating of behaviors of a person with ASD that occurred over the week prior to assessment. It contains 14 items reflecting the core and associated autism behavior domains: Social Communication, Restrictive Behaviors, Mood and Anxiety, Self Regulation, and Challenging Behavior. Each item is rated on a 7-point scale from 1 (none; no symptoms present) to 7 (very severe; persistent interference with function or adaptation).

Change from Baseline in Aberrant Behavior Checklist 2-Irritability (ABC-I) Subscale Score [Time Frame: Baseline Up to Day 110]

The ABC is a parent- or care/study partner-reported behavior rating assessment with five domains and 58 items, each rated on a 0 (not a problem) to 3 (the problem is severe in degree) scale. The irritability domain consists of 15 items.

Change from Baseline in the Clinician Global Impression of Severity (CGI-S) Score [Time Frame: Baseline Up to Day 110]

The CGI-S is a global assessment of the clinician-rater's impression of the severity of the participant's illness. It is rated on a scale of 1 (normal, not at all ill) to 7 (among the most extremely ill).

Change from Baseline in the ABI Repetitive/Restrictive Behavior Domain Score [Time Frame: Baseline Up to Day 110]

Each item on the ABI-Repetitive/Restrictive Behavior Domain is rated by frequency (0=never to 3=very often). The domain score is the sum of all domain items scores divided by the number of items in the domain.

Change from Baseline in the ABI Mood and Anxiety Domain Score [Time Frame: Baseline Up to Day 110]

Each item on the ABI-Mood and Anxiety Domain is rated by frequency (0=never to 3=very often). The domain score is the sum of all domain items scores divided by the number of items in the domain.

Change from Baseline in the ABI Challenging Behavior Domain Score [Time Frame: Baseline Up to Day 110]

Each item on the ABI-Challenging Behavior Domain is rated by frequency (0=never to 3=very often). The domain score is the sum of all domain items scores divided by the number of items in the domain.

Change from Baseline in the ABI Self-Regulation Domain Score [Time Frame: Baseline Up to Day 110]

Each item on the ABI-Self-regulation Domain is rated by frequency (0=never to 3=very often). The domain score is the sum of all domain items scores divided by the number of items in the domain.

Change from Baseline in the ABI-Short Form (ABI-S) Score [Time Frame: Baseline Up to Day 110]

The ABI-S is a 24-item short version of the ABI, containing items from each of the five domains. The domain score for each domain is calculated as the sum of scores all domain items divided by the number of items in the domain.

Change from Baseline in the ABC-Social Withdrawal (ABC-SW) Subscale Score [Time Frame: Baseline Up to Day 110]

The ABC-SW subscale consists of 16 items of the ABC-2 rated from 0 (not a problem) to 3 (the problem is severe in degree).

Change from Baseline in the Social Responsiveness Scale 2 (SRS-2) Score [Time Frame: Baseline Up to Day 110]

The SRS-2 consists of 65 items across 5 subscales. Responses range from 1 (not true) to 4 (almost always true).

Change from Baseline in the Vineland-3 (Domain Level Version) Score: Total of Communication, Socialization, and Maladaptive Behavior Domains [Time Frame: Baseline Up to Day 110]

The Vineland-3 Domain Level Version contains 5 domains. Responses on each item are rated from 0 (never) to 2 (usually).

### Eligibility Criteria

12 Years to 45 Years (Child, Adult)

### Inclusion Criteria:

Age 12 to 45 at screening
Has a body mass index (BMI) 18 through 34 kg/m₂, inclusive
Has a designated care/study partner who can reliably report on symptoms
Has a diagnosis of Autism Spectrum Disorder (ASD) according to American Psychiatric Association's (APA 2013) Diagnostic and Statistical Manual, 5th ed (DSM-5) and/or the World Health Organization (WHO) International Classification of Diseases External 10th Revision, 2nd ed (ICD-10, WHO 2004) and diagnosis has been confirmed with the Autism Diagnostic Observation Scale (ADOS, Lord 1999; or ADOS-2, Lord 2012) or Autism Diagnostic Interview-Revised (ADI-R, Lord 1994) obtained within the preceding 3 years, or the ADOS-2 performed at screening. Full scale IQ (or equivalent) 270 score.
Has an Aberrant Behavior Checklist 2-Social Withdrawal (ABC-SW) subscale score≥11 at Screening.
Psychoactive medications and adjunctive therapies are stable for 4 weeks prior to screening.
Must be able to swallow study medication.
Male subjects and female subjects of childbearing potential who are sexually active must practice effective contraception from time of screening through 30 days after their last dose of study drug. Effective contraception is the use of 2 contraception methods, defined as condom use (male and/or female type), hormonal contraception (women) or intrauterine device (IUD). This does not apply to subjects who are surgically sterilized by bilateral tubal ligation, bilateral oophorectomy, or hysterectomy; or subjects who practice sexual abstinence while a research subject in this study; or subjects in same-sex relationships.

### Exclusion Criteria:

Has Rett syndrome or Child Disintegrative Disorder
Has participated in any other study and received any other investigational medication (other than COVID-19 vaccination) or device within 60 days prior to screening
History of epilepsy without current adequate control, or any seizure in the 6 months preceding screening
History of suicidal ideation or behavior in the past 12 months, or a positive response to C-SSRS questions 4 and/or 5
Systolic blood pressure >160 mmHg, or diastolic blood pressure >100, or a clinical history of uncontrolled or severe hypertension
If female, is pregnant or lactating
Have taken, within 2 weeks (or 5 half-lives, whichever is longer) of single-blind baseline (Visit 2), any of the following:
Selective serotonin reuptake inhibitors (SSRIs)
Serotonin-norepinephrine reuptake inhibitors (SNRIs)
Tricyclic antidepressants (TCAs)
Any monoamine oxidase, type A (MAO-A) inhibitors
Another 5-HT1 agonist or antagonist (e.g., risperidone), partial agonist/antagonist (e.g., aripiprazole)
Any ergotamine-containing or ergot-type medication (e.g., dihydroergotamine or methysergide), including St John's wort
Is taking cimetidine and is unable to discontinue use of cimetidine during screening and through the end of study
Has a diagnosis or clinical history of coronary artery disease, coronary vasospasm, Prinzmetal's angina, Wolff-Parkinson-White syndrome, peripheral vascular disease, stroke, transient ischemic attack, ischemic bowel disease, or other significant cardiac or cerebrovascular disease 3
Has a diagnosis of, or clinical history indicative of migraines with or without aura, including basilar and/or hemiplegic migraine
Has a history of galactose intolerance (i.e., Lapp lactase deficiency or glucose-galactose malabsorption)
Has participated in any other study and received any other investigational medication (other than COVID-19 vaccination) or device within 60 days prior to screening, or is taking part in a non-medication study which, in the opinion of the Investigator, would interfere with the interpretation of the assessments in this study
History of suicidal ideation or behavior in the past 12 months, or a positive response to C-SSRS questions 4 and/or 5 (current or over last 6 months) at the screening or single-blind baseline (Visit 2) assessments, and/or is a significant risk for suicidal behavior, in the opinion of the Investigator
Has a screening or single-blind baseline (Visit 2) systolic blood pressure >140 mmHg (if adult) or >135 mmHg (if adolescent), or diastolic blood pressure 290 mmHg (if adult) or >85 mmHg (if adolescent) or a clinical history of uncontrolled or severe hypertension. For the subjects with automated or manual blood pressures in the more mildly abnormal range (SBP 140-159 mmHg, DBP 90-99 mmHg for adults and SBP 135-140 mmHg, DBP 85-89 mmHg for adolescents), the protocol does not require absolute exclusion, but medical monitors would require further data (per guidelines) for accurate determination of a subject's blood pressure status in order to determine eligibility. In these cases, if either hypertension is excluded or a cause is identified and can be adequately treated, the subject may be eligible to participate if the Medical Monitor has reviewed and granted permission to do so.
Has clinically significant ECG abnormalities at screening or single-blind baseline (Visit 2), in the clinical judgement of the Investigator
Has one of the following screening laboratory results: a. Platelets ≤75,000/mm3
b. Neutrophils, absolute, <1000/mm3
c. Alanine aminotransferase (ALT) or aspartate aminotransferase (AST) >2 times the upper limit of normal and/or total bilirubin >1 times the upper limit of normal
d. Creatinine ≥2 mg/dL and/or eGFR <60 mL/min/1.73m2 (adult) or <75 mL/min/1.73m2 (adolescent)
e. Abnormal free thyroxine (T4)
Laboratory testing may be repeated during screening at the discretion of the Medical Monitor.
Has a history of alcohol use or substance use disorder (by DSM-5 criteria) within 12 months of screening or a positive screen for drugs of abuse at screening (unless consistent with current prescription for medical condition).
Positive screening for human immunodeficiency virus antibodies, hepatitis B surface antigen, or hepatitis C virus antibodies at screening
If female, is pregnant or lactating
Diagnosis or clinical history consistent with schizophrenia, bipolar disorder, or other medical or psychiatric condition which, in the opinion of the Investigator or Medical Monitor, would place the subject at increased risk of safety/tolerability issues, and/or would preclude obtaining voluntary consent, and/or would confound the interpretation of the primary outcome measures in the study
Is unwilling or unable to comply with the study protocol (including the inability of swallowing the investigational product), for any reason.
Has significant visual, auditory, or motor impairments that would preclude participation in completion of key assessments.
Is judged to be inappropriate for the study for any reason by the Investigator or Medical Monitor.

### EMBODIMENTS

1. An oral composition for treating the symptoms of autism spectrum disorder or aggression in an Alzheimer's patient, wherein the composition comprises from about 7.5 mg to about 90 mg of zolmitriptan, or a pharmaceutical acceptable salt thereof, and the oral administration of the composition provides a therapeutically effective plasma concentration of zolmitriptan for a period of at least 8 hours.

1a. An oral composition for treating the symptoms of aggression in a patient with dementia, wherein the composition comprises from about 7.5 mg to about 90 mg of zolmitriptan, or a pharmaceutical acceptable salt thereof, and the oral administration of the composition provides a therapeutically effective plasma concentration of zolmitriptan for a period of at least 8 hours.

2. The composition of claim 1-1a, wherein the composition comprises about 10 mg to about 30 mg of zolmitriptan or a pharmaceutical acceptable salt thereof.

3. The composition of any one of claims 1-2, wherein the composition comprises an immediate release portion and an extended release portion.

4. The composition of claim 3, wherein the immediate release portion contains about 20%-40% by weight of the total zolmitriptan in the composition.

5. The composition of any one of claims 3-4, wherein the extended release portion contains about 60%-80% by weight of the total zolmitriptan in the composition.

6. The composition of any one of claims 3-5, wherein the immediate release portion contains about 25% of the total zolmitriptan in the composition and the extended release portion contains about 75% of the total zolmitriptan in the composition.

7. The composition of any one of claims 3-6, wherein the immediate release portion comprises about 1 mg to about 10 mg of zolmitriptan.

8. The composition of claim 7, wherein the immediate release portion comprises about 3 mg of zolmitriptan.

9. The composition of claim 7, wherein the immediate release portion comprises about 6 mg of zolmitriptan.

10. The composition of any one of claims 3-9, wherein the extended release portion comprises about 5 mg to about 25 mg of zolmitriptan.

11. The composition of claim 10, wherein the extended release portion comprises 9 mg of zolmitriptan.

12. The composition of claim 10, wherein the extended release portion comprises about 18 mg of zolmitriptan.

13. The composition of any one of claims 1-12, wherein the oral administration of the composition provides a Cₘₐₓ of about 3 ng/mL to about 30 ng/mL following a single administration of the composition.

14. The composition of any one of claims 1-13, wherein the oral administration of the composition provides a blood plasma concentration of at least about 10 ng/mL for at least 8 hours following a single administration of the composition.

15. The composition of any one of claims 1-14 wherein the oral administration of the composition provides an AUC₀₋₂₄ₕ of about 40 ng·h/mL to about 300 ng·h/mL following a single administration of the composition.

16. The composition of any one of claims 1-15, wherein the oral administration of the composition provides an AUC₀₋₂₄ₕ of about 50 ng·h/mL to about 250 ng·h/mL following a single administration of the composition.

17. The composition of any one of claims 1-15, wherein the oral administration of the composition provides an AUC₀₋₂₄ₕ of about 150 ng·h/mL to about 250 ng·h/mL following a single administration of the composition.

18. The composition of any one of claims 1-15, wherein the oral administration of the composition an AUC₀₋₂₄ₕ of about 50 ng·h/mL to about 150 ng·h/mL following a single administration of the composition.

19. The composition of any one of claims 1-18, wherein the oral administration of the composition provides a therapeutically effective plasma concentration of zolmitriptan for a period of about 12 h following a single administration of the composition.

20. The composition of any one of claims 1-19, wherein the oral administration of the composition provides a therapeutically effective plasma concentration of zolmitriptan for a period of about 18 h following a single administration of the composition.

21. The composition of any one of claims 1-20, wherein the oral administration of the composition a therapeutically effective plasma concentration of zolmitriptan for a period of about 24 h following a single administration of the composition.

22. The composition of any one of claims 1-21, wherein the composition is a multiparticulate formulation.

23. The composition of claim 3-21, wherein the extended release portion comprises drug-containing particles coated with an extended release coating.

24. The composition of any one of claims 1-21, wherein the composition is a gastrorentive tablet.

25. The composition of any one of claims 3-21, wherein the extended release portion is a gastrorentive layer.

26. The composition of claim 25, wherein the gastroretentive layer comprises a water-swellable polymer.

27. The composition of claim 25, wherein the water-swellable polymer is selected from the group consisting of polyalkylene oxides, cellulose polymers and derivatives thereof, polysaccharides and derivatives thereof, chitosan, poly(vinyl alcohol), xanthan gum, maleic anhydride copolymers, poly(vinyl pyrrolidone), starch and starch-based polymers; maltodextrin, poly (2-ethyl-2-oxazoline), poly(ethyleneimine), polyurethane, hydrogels, crosslinked polyacrylic acids, and combinations thereof.

28. The composition of claim 25, wherein the water-swellable polymer is selected from the group consisting of high molecular weight polyethylene oxide, hydroxyalkyl cellulose, hydroxymethyl cellulose, hydroxyethyl cellulose, hydroxypropyl cellulose, hydroxypropylmethyl cellulose, carboxymethylcellulose, and microcrystalline cellulose.

29. The composition of claim 28, wherein the water swellable polymer is polyethylene oxide.

30. The composition of any one of claims 26-29, wherein the composition comprises about 35-55% by weight of polyethylene oxide.

31. The composition of any one claims 24-30, wherein the composition is gastroretained for at least 2 h following oral administration.

32. The composition of any one claims 24-31, wherein the composition is gastroretained for at least 8 h following oral administration.

33. An oral composition of zolmitriptan, or a pharmaceutically acceptable salt thereof for treating the symptoms of autism spectrum disorder (or aggression in an Alzheimer's patient) providing a modified release profile,
wherein the composition when dissolution tested using United States Pharmacopoeia Apparatus II (paddles @ 50 rpm) in 900 mL of 0.1N HCl at 37° C. exhibits a zolmitriptan release profile substantially corresponding to the following pattern:
from about 20%-40% of the total zolmitriptan is released after about 15 minutes;
after 4 h about 40%-75% of the total zolmitriptan is released; and
after 8 h about 75%-90% of the total zolmitriptan is released.

34. The composition of claim 33, wherein about 85% of the total zolmitriptan is released within 9-10 hours.

35. The composition of any one of claims 33-34, wherein the composition comprises about 10 mg to about 30 mg of zolmitriptan or a pharmaceutical acceptable salt thereof.

36. The composition of any one of claims 33-35, wherein the composition comprises an immediate release portion and an extended release portion.

37. The composition of claim 36, wherein the immediate release portion contains about 20%-40% by weight of the total zolmitriptan in the composition.

38. The composition of any one of claims 36-37, wherein the extended release portion contains about 60%-80% by weight of the total zolmitriptan in the composition.

39. The composition of any one of claims 36-38, wherein the immediate release portion comprises about 1 mg to about 10 mg of zolmitriptan.

40. The composition of any one of claims 36-39, wherein the extended release portion comprises about 5 mg to about 25 mg of zolmitriptan.

41. The composition of any one of claims 33-40, wherein the oral administration of the composition provides a Cₘₐₓ of about 3 ng/mL to about 30 ng/mL following a single administration of the composition.

42. The composition of any one of claims 33-40, wherein the oral administration of the composition provides an AUC₀₋₂₄ₕ of about 40 ng·h/mL to about 300 ng·h/mL following a single administration of the composition.

43. The composition of any one of claims 33-42, wherein the composition is a gastroretentive tablet.

44. The composition of any one of claims 33-43, wherein the extended release portion is a gastrorentive layer.

45. The composition of claim 44, wherein the gastroretentive layer comprises a water-swellable polymer.

46. The composition of claim 45, wherein the water-swellable polymer is selected from the group consisting of polyalkylene oxides, cellulose polymers and derivatives thereof, polysaccharides and derivatives thereof, chitosan, poly(vinyl alcohol), xanthan gum, maleic anhydride copolymers, poly(vinyl pyrrolidone), starch and starch-based polymers; maltodextrin, poly (2-ethyl-2-oxazoline), poly(ethyleneimine), polyurethane, hydrogels, crosslinked polyacrylic acids, and combinations thereof.

47. The composition of claim 45, wherein the water-swellable polymer is selected from the group consisting of high molecular weight polyethylene oxide, hydroxyalkyl cellulose, hydroxymethyl cellulose, hydroxyethyl cellulose, hydroxypropyl cellulose, hydroxypropylmethyl cellulose, carboxymethylcellulose, and microcrystalline cellulose.

48. The composition of claim 45, wherein the water swellable polymer is polyethylene oxide.

49. The composition of any one of claims 46-48, wherein the composition comprises about 35-55% by weight of polyethylene oxide.

50. The composition of any one of claims 33-49, wherein the oral administration of the composition provides a therapeutically effective plasma concentration of zolmitriptan for a period of about 12 h following a single administration of the composition.

51. The composition of any one of claims 33-42, wherein the composition is a multiparticulate formulation.

52. The composition of claim 33-42, wherein the extended release portion comprises drug-containing particles coated with an extended release coating.

53. A composition for treating the symptoms of autism spectrum disorder, wherein the composition comprises zolmitriptan, or a pharmaceutically acceptable salt thereof, wherein the oral administration of the composition provides an AUC₀₋₂₄ₕ of about 40 ng·h/mL to about 300 ng·h/mL to treat the symptoms of autism spectrum disorder.

54. The composition of claim 53, wherein the oral administration of the composition provides an AUC₀₋₂₄ₕ of about 40 ng·h/mL to about 110 ng·h/mL following a single administration of the composition.

55. The composition of claim 53, wherein the oral administration of the composition provides an AUC_{0-24h,ss} of about 150 ng·h/mL to about 450 ng·h/mL following a single administration of the composition.

56. The composition of any one of claims 53-55, wherein the oral administration of the composition provides a therapeutically effective plasma concentration of zolmitriptan for a period of about 12 h following a single administration of the composition.

57. A method of treating the symptoms of autism spectrum disorder, comprising orally administering a pharmaceutical composition of a composition of any one of claims 1-56 to a patient in need thereof.

58. A composition for treating aggression in an Alzheimer's patient, wherein the composition comprises zolmitriptan, or a pharmaceutically acceptable salt thereof, wherein the oral administration of the composition provides an AUC₀₋₂₄ₕ of about 40 ng·h/mL to about 300 ng·h/mL to treat the patient's aggression.

59. The composition of claim 58, wherein the oral administration of the composition provides an AUC₀₋₂₄ₕ of about 40 ng·h/mL to about 110 ng·h/mL following a single administration of the composition.

60. The composition of claim 58, wherein the oral administration of the composition provides an AUC_{0-24h,ss} of about 150 ng·h/mL to about 450 ng·h/mL following a single administration of the composition.

61. The composition of any one of claims 58-60, wherein the oral administration of the composition provides a therapeutically effective plasma concentration of zolmitriptan for a period of about 12 h following a single administration of the composition.

62. A method of treating aggression in an Alzheimer's patient, comprising orally administering a pharmaceutical composition of a composition of any one of claims 1-56 to a patient in need thereof.

63. A composition for treating aggression in a patient with dementia, wherein the composition comprises zolmitriptan, or a pharmaceutically acceptable salt thereof, wherein the oral administration of the composition provides an AUC₀₋₂₄ₕ of about 40 ng·h/mL to about 300 ng·h/mL to treat the patient's aggression.

64. The composition of claim 63, wherein the oral administration of the composition provides an AUC₀₋₂₄ₕ of about 40 ng·h/mL to about 110 ng·h/mL following a single administration of the composition.

65. The composition of claim 63, wherein the oral administration of the composition provides an AUC_{0-24h,ss} of about 150 ng·h/mL to about 450 ng·h/mL following a single administration of the composition.

66. The composition of any one of claims 63-65, wherein the oral administration of the composition provides a therapeutically effective plasma concentration of zolmitriptan for a period of about 12 h following a single administration of the composition.

67. A method of treating aggression a patient with dementia, comprising orally administering a pharmaceutical composition of a composition of any one of claims 1-56 to a patient in need thereof.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

What is claimed is:

1. An oral composition for treating the symptoms of autism spectrum disorder, wherein the composition comprises from about 7.5 mg to about 90 mg of zolmitriptan, or a pharmaceutical acceptable salt thereof, and the oral administration of the composition provides a therapeutically effective plasma concentration of zolmitriptan for a period of at least 8 hours.

2. The composition of claim 1, wherein the composition comprises about 10 mg to about 30 mg of zolmitriptan or a pharmaceutical acceptable salt thereof.

3. The composition of any one of claims 1-2, wherein the composition comprises an immediate release portion and an extended release portion.

4. The composition of claim 3, wherein the immediate release portion contains about 20%-40% by weight of the total zolmitriptan in the composition.

5. The composition of any one of claims 3-4, wherein the extended release portion contains about 60%-80% by weight of the total zolmitriptan in the composition.

6. The composition of any one of claims 3-5, wherein the immediate release portion contains about 25% of the total zolmitriptan in the composition and the extended release portion contains about 75% of the total zolmitriptan in the composition.

7. The composition of any one of claims 3-6, wherein the immediate release portion comprises about 1 mg to about 10 mg of zolmitriptan.

8. The composition of claim 7, wherein the immediate release portion comprises about 3 mg of zolmitriptan.

9. The composition of claim 7, wherein the immediate release portion comprises about 6 mg of zolmitriptan.

10. The composition of any one of claims 3-9, wherein the extended release portion comprises about 5 mg to about 25 mg of zolmitriptan.

11. The composition of claim 10, wherein the extended release portion comprises 9 mg of zolmitriptan.

12. The composition of claim 10, wherein the extended release portion comprises about 18 mg of zolmitriptan.

13. The composition of any one of claims 1-12, wherein the oral administration of the composition provides a Cₘₐₓ of about 3 ng/mL to about 30 ng/mL following a single administration of the composition.

14. The composition of any one of claims 1-13, wherein the oral administration of the composition provides a blood plasma concentration of at least about 10 ng/mL for at least 8 hours following a single administration of the composition.

15. The composition of any one of claims 1-14 wherein the oral administration of the composition provides an AUC₀₋₂₄ₕ of about 40 ng·h/mL to about 300 ng·h/mL following a single administration of the composition.

16. The composition of any one of claims 1-15, wherein the oral administration of the composition provides an AUC₀₋₂₄ₕ of about 50 ng·h/mL to about 250 ng·h/mL following a single administration of the composition.

17. The composition of any one of claims 1-15, wherein the oral administration of the composition provides an AUC₀₋₂₄ₕ of about 150 ng·h/mL to about 250 ng·h/mL following a single administration of the composition.

18. The composition of any one of claims 1-15, wherein the oral administration of the composition an AUC₀₋₂₄ₕ of about 50 ng·h/mL to about 150 ng·h/mL following a single administration of the composition.

19. The composition of any one of claims 1-18, wherein the oral administration of the composition provides a therapeutically effective plasma concentration of zolmitriptan for a period of about 12 h following a single administration of the composition.

20. The composition of any one of claims 1-19, wherein the oral administration of the composition provides a therapeutically effective plasma concentration of zolmitriptan for a period of about 18 h following a single administration of the composition.

21. The composition of any one of claims 1-20, wherein the oral administration of the composition a therapeutically effective plasma concentration of zolmitriptan for a period of about 24 h following a single administration of the composition.

22. The composition of any one of claims 1-21, wherein the composition is a multiparticulate formulation.

23. The composition of claim 3-21, wherein the extended release portion comprises drug-containing particles coated with an extended release coating.

24. The composition of any one of claims 1-21, wherein the composition is a gastrorentive tablet.

25. The composition of any one of claims 3-21, wherein the extended release portion is a gastrorentive layer.

26. The composition of claim 25, wherein the gastroretentive layer comprises a water-swellable polymer.

27. The composition of claim 25, wherein the water-swellable polymer is selected from the group consisting of polyalkylene oxides, cellulose polymers and derivatives thereof, polysaccharides and derivatives thereof, chitosan, poly(vinyl alcohol), xanthan gum, maleic anhydride copolymers, poly(vinyl pyrrolidone), starch and starch-based polymers; maltodextrin, poly (2-ethyl-2-oxazoline), poly(ethyleneimine), polyurethane, hydrogels, crosslinked polyacrylic acids, and combinations thereof.

28. The composition of claim 25, wherein the water-swellable polymer is selected from the group consisting of high molecular weight polyethylene oxide, hydroxyalkyl cellulose, hydroxymethyl cellulose, hydroxyethyl cellulose, hydroxypropyl cellulose, hydroxypropylmethyl cellulose, carboxymethylcellulose, and microcrystalline cellulose.

29. The composition of claim 28, wherein the water swellable polymer is polyethylene oxide.

30. The composition of any one of claims 26-29, wherein the composition comprises about 35-55% by weight of polyethylene oxide.

31. The composition of any one claims 24-30, wherein the composition is gastroretained for at least 2 h following oral administration.

32. The composition of any one claims 24-31, wherein the composition is gastroretained for at least 8 h following oral administration.

33. An oral composition of zolmitriptan, or a pharmaceutically acceptable salt thereof for treating the symptoms of autism spectrum disorder providing a modified release profile,
wherein the composition when dissolution tested using United States Pharmacopoeia Apparatus II (paddles @ 50 rpm) in 900 mL of 0.1N HCl at 37° C. exhibits a zolmitriptan release profile substantially corresponding to the following pattern:
from about 20%-40% of the total zolmitriptan is released after about 15 minutes;
after 4 h about 40%-75% of the total zolmitriptan is released; and
after 8 h about 75%-90% of the total zolmitriptan is released.

34. The composition of claim 33, wherein about 85% of the total zolmitriptan is released within 9-10 hours.

35. The composition of any one of claims 33-34, wherein the composition comprises about 10 mg to about 30 mg of zolmitriptan or a pharmaceutical acceptable salt thereof.

36. The composition of any one of claims 33-35, wherein the composition comprises an immediate release portion and an extended release portion.

37. The composition of claim 36, wherein the immediate release portion contains about 20%-40% by weight of the total zolmitriptan in the composition.

38. The composition of any one of claims 36-37, wherein the extended release portion contains about 60%-80% by weight of the total zolmitriptan in the composition.

39. The composition of any one of claims 36-38, wherein the immediate release portion comprises about 1 mg to about 10 mg of zolmitriptan.

40. The composition of any one of claims 36-39, wherein the extended release portion comprises about 5 mg to about 25 mg of zolmitriptan.

41. The composition of any one of claims 33-40, wherein the oral administration of the composition provides a Cₘₐₓ of about 3 ng/mL to about 30 ng/mL following a single administration of the composition.

42. The composition of any one of claims 33-40, wherein the oral administration of the composition provides an AUC₀₋₂₄ₕ of about 40 ng·h/mL to about 300 ng·h/mL following a single administration of the composition.

43. The composition of any one of claims 33-42, wherein the composition is a gastroretentive tablet.

44. The composition of any one of claims 33-43, wherein the extended release portion is a gastrorentive layer.

45. The composition of claim 44, wherein the gastroretentive layer comprises a water-swellable polymer.

46. The composition of claim 45, wherein the water-swellable polymer is selected from the group consisting of polyalkylene oxides, cellulose polymers and derivatives thereof, polysaccharides and derivatives thereof, chitosan, poly(vinyl alcohol), xanthan gum, maleic anhydride copolymers, poly(vinyl pyrrolidone), starch and starch-based polymers; maltodextrin, poly (2-ethyl-2-oxazoline), poly(ethyleneimine), polyurethane, hydrogels, crosslinked polyacrylic acids, and combinations thereof.

47. The composition of claim 45, wherein the water-swellable polymer is selected from the group consisting of high molecular weight polyethylene oxide, hydroxyalkyl cellulose, hydroxymethyl cellulose, hydroxyethyl cellulose, hydroxypropyl cellulose, hydroxypropylmethyl cellulose, carboxymethylcellulose, and microcrystalline cellulose.

48. The composition of claim 45, wherein the water swellable polymer is polyethylene oxide.

49. The composition of any one of claims 46-48, wherein the composition comprises about 35-55% by weight of polyethylene oxide.

50. The composition of any one of claims 33-49, wherein the oral administration of the composition provides a therapeutically effective plasma concentration of zolmitriptan for a period of about 12 h following a single administration of the composition.

51. The composition of any one of claims 33-42, wherein the composition is a multiparticulate formulation.

52. The composition of claim 33-42, wherein the extended release portion comprises drug-containing particles coated with an extended release coating.

53. A composition for treating the symptoms of autism spectrum disorder, wherein the composition comprises zolmitriptan, or a pharmaceutically acceptable salt thereof, wherein the oral administration of the composition provides an AUC₀₋₂₄ₕ of about 40 ng·h/mL to about 300 ng·h/mL to treat the symptoms of autism spectrum disorder.

54. The composition of claim 53, wherein the oral administration of the composition provides an AUC₀₋₂₄ₕ of about 40 ng·h/mL to about 110 ng·h/mL following a single administration of the composition.

55. The composition of claim 53, wherein the oral administration of the composition provides an AUC_{0-24h,ss} of about 150 ng·h/mL to about 450 ng·h/mL following a single administration of the composition.

56. The composition of any one of claims 53-55, wherein the oral administration of the composition provides a therapeutically effective plasma concentration of zolmitriptan for a period of about 12 h following a single administration of the composition.

57. A method of treating the symptoms of autism spectrum disorder, comprising orally administering a pharmaceutical composition of a composition of any one of claims 1-56 to a patient in need thereof.
